(19) United States
(12) Patent Application Publication
(10) Pub. No.: US 2023/0183296 A1
(43) Pub. Date: Jun. 15, 2023

(54) COMPOSITIONS AND METHODS FOR REDUCING HOST REJECTION OF ALLOGENEIC CELLS USING SIMIAN ICP47 AND VARIANTS THEREOF

(71) Applicant: Nanjing Legend Biotech Co., Ltd., Nanjing (CN)

(72) Inventors: Ming ZENG, Irving, TX (US); Huihui ZHANG, Nanjing (CN)

(21) Appl. No.: 17/924,253

(22) PCT Filed: May 13, 2021
(86) PCT No.: PCT/CN2021/093508

§ 371 Date: Nov. 9, 2022

(30) Foreign Application Priority Data
May 13, 2020 (WO) PCT/CN2020/090069

(51) Int. Cl. C07K 14/035; C07K 14/705; A61P 37/06

(52) U.S. Cl. CPC C07K 14/035; C07K 14/705; A61P 37/06; C07K 2317/565

(57) ABSTRACT

Provided are modified therapeutic cells comprising a first heterologous nucleic acid sequence encoding a simian ICP47 (sICP47) protein or a functional variant thereof. In some embodiments, the modified therapeutic cell further comprises a second heterologous nucleic acid sequence encoding an agonist of a natural killer cell inhibitory receptor. Also provided are methods of treatment using the modified therapeutic cells.

### CROSS REFERENCE TO RELATED APPLICATION

This application claims the benefit of International Application No. PCT/ICN2020/090069, filed May 13, 2020, which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 761422002541SEQLIST.TXT, date recorded: May 3, 2021, size: 23 KB).

### FIELD OF THE PRESENT APPLICATION

The present application generally relates to immunotherapy, and more specifically to the compositions and methods for reducing undesired immune response in adoptive cell therapy.

### BACKGROUND OF THE PRESENT APPLICATION

Adoptive cell therapy or adoptive cell transfer (ACT) is becoming an ever more important treatment paradigm, particularly in the treatment of cancer. ACT refers to the transfer of therapeutic cells, most typically immune cells, into a patient. These cells may have originated from the patient (i.e., autologous therapy) or from another individual of the same species (i.e., allogeneic therapy). The goal of ACT is to improve functions and characteristics of the immune system in the patient. Specially, in cancer immunotherapy, the goal of ACT is to trigger an immune response against the cancer. Although T cells are most often used in ACT, other immune cell types such as NK cells, lymphocytes (e.g., tumor-infiltrating lymphocytes or TILs), dendritic cells and myeloid cells have also been applied.

Ideally, the therapeutic cells infused to a patient receiving an ACT (or reinfused in case of autologous therapy) will expand and persist in the patient. However, the immune system has developed elaborate and effective mechanisms against foreign agents. The patient's immune response presents the most formidable barriers against successful ACT, especially allogeneic ACT. One barrier is graft-versus-host disease (GvHD), in which donor T cells recognize a patient's cells as foreign, resulting in attack of the patient's healthy tissue and organs. It can be fatal even in the case of HLA-matched donors, as minor mismatches can still cause a reaction, which is a major complication of HSCT. Another barrier is the rejection of universal cell products by the recipient's immune system. Universal cell products are not HLA-matched with the recipient, and are recognized as foreign bodies. Thus, allogeneic CAR-T cell therapy faces significant challenges in the clinic, including poor in vivo persistence of allogeneic transferred cells, short-term efficacy, and lower-than-expected objective response rates (e.g., partial response or complete response rates) among patients.

Alloreactive T cells play a major role in the recognition of allogeneic graft cells and the immune response that results in graft rejection. A foreign cell can be recognized by a single T cell receptor (TCR) dues to the amino acid polymorphisms of its allogeneic MHC molecule, which activates cytotoxic CD8 T-cell responses against the graft cells. On the other hand, alloreactive natural killer (NK) cells also play a key role in immune response mechanisms elicited by the allograft. NK cells complement T cell immunity by killing infected and transformed cells that down-regulate MHC-I to evade MHC-I-restricted T cells (see Totterman et al., Transplantation 1989; 47:817-823). It has been shown that NK cells cause rejection in kidney allografts, and donor blood cells (see Nowak 1. et al., PLoS ONE 2012; 7(9):e44718; and Vampa et al., Transplantation 2003; 7:1220-1228.)

Strategies have been developed to suppress the host immune system in order to prolong in vivo persistence of allogeneic CAR-T cells. In order to prevent GvHD, gene editing method is used in clinical practice to knock out TCR of T cells (Graham C et al., Cells 2018 7(10); Yinmeng Y et al., Curr Opin. Hematol. 2015 22(6): 509-515; Pavan R. et al., Hematology Am Soc Hematol Educ Program. 2015:225-30). In order to prevent host cells from rejecting allogeneic cells, there are clinically programs that use high-intensity lymphatic removal to allow cells derived from healthy donors to expand and clear malignant cells before host immunity is restored. As design of Allogene Therapeutics's lead product, UCART19, an allogeneic CAR-T (anti-CD19 scFv-41BB-CD3ζ) manufactured from healthy donor T cells, in which TRAC and CD52 genes have been knocked out to allow its administration in non-HLA matched patients. Another gene editing method is to remove HLA class I and II molecules from expression on CAR-T cells. For example, by knocking out HLLA class I molecules, the cell membrane protein that is essential for the expression of β-2 microglobulin (B2M), can prevent host cells from recognizing donor CAR-T cells as heterologous through class I HLA (see Piorot, L. et al., Cancer Res. 2015, 75, 3853-3864; and Torikai, H. et al., Blood 2013, 122, 1341-1349). One of the issues with HLA class I knockout is that activated T cells also express class 11 HLA, which may still stimulate rejection. Even worse, after the HLA molecule is knocked out, the sensitivity of NK cells to cells without HLA expression will be greatly increased. Much shorter persistency of allo-CART is a significant unsolved bottleneck for allo-product to be used as monotherapy. In addition, genome editing of clinically relevant human somatic cells remains a challenge, and the genome-edited cells may introduce additional sources of immunogenicity. There remains a need for effective strategies to reduce undesired immune response associated with allogeneic cell therapy, which do not require genome modification of the therapeutic cells.

The disclosures of all publications, patents, patent applications and published patent applications referred to herein are hereby incorporated herein by reference in their entirety.

### BRIEF SUMMARY OF THE INVENTION

The present application provides modified therapeutic cells expressing a viral protein such as simian ICP47 and methods of use thereof.

One aspect of the present application provides a modified therapeutic cell comprising a first heterologous nucleic acid sequence encoding a simian ICP47 (sICP47) protein or a functional variant thereof.

Another aspect of the present application provides a method of reducing graft rejection of allogeneic therapeutic cells in an individual in need thereof, comprising administering to the individual an effective amount of the allogeneic therapeutic cells, wherein the allogeneic therapeutic cells comprise a first heterologous nucleic acid sequence encoding a sICP47 protein or functional variant thereof.

In some embodiments according to any one of the modified therapeutic cells or methods described above, the first heterologous nucleic acid sequence encodes a naturally occurring sICP47 protein. In some embodiments, the first heterologous nucleic acid sequence encodes an engineered sICP47 protein comprising an active domain of a naturally occurring sICP47 protein. In some embodiments, the naturally occurring sICP47 protein is from a herpesvirus selected from the group consisting of Simian Agent 8 (SA8), Cercopithecine herpesvirus 16 (CeHV-16) and Cercopithecine herpesvirus 1 (CeHV-1). In some embodiments, the first heterologous nucleic acid sequence encodes a sICP47 of SA8 or a functional variant thereof. In some embodiments, the first heterologous nucleic acid sequence encodes a sICP47 of CeHV-16 or a functional variant thereof. In some embodiments, the first heterologous nucleic acid sequence encodes a sICP47 of CeHV-1 or a functional variant thereof

In some embodiments according to any one of the modified therapeutic cells or methods described above, the sICP47 protein or functional variant thereof comprises the amino acid sequence having at least about 80% (e.g., at least about any one of 85%, 90%, 95%, 98%, 99% or more) sequence identity to an amino acid sequence selected from the group consisting of SEQ ID NOs: 1-3. In some embodiments, the sICP47 protein or functional variant thereof comprises the amino acid sequence of SEQ ID NO: 1. In some embodiments, the sICP47 protein or functional variant thereof comprises the amino acid sequence of SEQ ID NO: 2. In some embodiments, the sICP47 protein or functional variant thereof comprises the amino acid sequence of SEQ ID NO: 3. In some embodiments, the sICP47 protein or functional variant thereof comprises the amino acid sequence having at least about 80% (e.g., at least about any one of 85%, 90%, 95%, 98%, 99% or more) sequence identity to an amino acid sequence selected from the group consisting of SEQ ID NOs: 4-6, 24 and 25. In some embodiments, the first heterologous nucleic acid sequence encodes a sICP47 protein comprising the amino acid sequence of SEQ ID NO: 4. In some embodiments, the first heterologous nucleic acid sequence encodes a sICP47 protein comprising the amino acid sequence of SEQ ID NO: 5, In some embodiments, the first heterologous nucleic acid sequence encodes a sICP47 protein comprising the amino acid sequence of SEQ ID NO: 6.

In some embodiments according to any one of the modified therapeutic cells or methods described above, the modified therapeutic cell further comprises a second heterologous nucleic acid sequence encoding an agonist agent of a natural killer (NK) cell inhibitory receptor. In some embodiments, the NK cell inhibitory receptor is an NKG2A receptor. In some embodiments, the agonist agent is a ligand of the NK cell inhibitory receptor. In some embodiments, the agonist agent is an agonist antibody or antigen binding fragment thereof that specifically binds to the NK cell inhibitory receptor. In some embodiments, the agonist antibody or antigen binding fragment thereof is membrane bound. In some embodiments, the agonist antibody or antigen binding fragment thereof specifically binds to NKG2A. In some embodiments, the agonist antibody or antigen binding fragment thereof comprises: (i) a heavy chain variable region (VH) comprising a heavy chain complementary determining region (HC-CDR) 1 comprising the amino acid sequence of SEQ ID NO: 7, an HC-CDR2 comprising the amino acid sequence of SEQ ID NO: 8, and an HC-CDR3 comprising the amino acid sequence of SEQ ID NO: 9; and (ii) a light chain variable region (VL) comprising a light chain complementary determining region (LC-CDR) 1 comprising the amino acid sequence of SEQ ID NO: 10, an LC-CDR2 comprising the amino acid sequence of SEQ ID NO: 11, and an LC-CDR3 comprising the amino acid sequence of SEQ ID NO: 12. In some embodiments, the agonist antibody or antigen binding fragment thereof comprises a VH comprising an amino acid sequence having at least about 85% sequence identity to the amino acid sequence of SEQ ID NO: 13, and a VL comprising an amino acid sequence having at least about 85% sequence identity to the amino acid sequence of SEQ ID NO: 14. In some embodiments, the agonist agent is a polypeptide comprising from the N-terminus to the C-terminus a single-chain variable fragment (scFv) that specifically binds to NKG2A, a CD8a hinge sequence and a CD8α. transmembrane sequence. In some embodiments, the agonist agent comprises an amino acid sequence having at least about 85% sequence identity to the amino acid sequence of SEQ ID NO: 15. In some embodiments, the agonist agent comprises an amino acid sequence having at least about 85% sequence identity to the amino acid sequence of SEQ ID NO: 16.

In some embodiments according to any one of the modified therapeutic cells or methods described above, the therapeutic cell is an immune cell. In some embodiments, the immune cell is selected from the group consisting of cytotoxic T cell, helper T cell, NK cell, NK-T cell, αβ T cell, γδ T cell, tumor-infiltrating T cell, dendritic cell (DC)-activated T cell, and peripheral blood mononuclear cell (PBMC). In some embodiments, the therapeutic cell is a T cell. In some embodiments, the therapeutic cell is a γδ T cell. In some embodiments, the therapeutic cell is a γ9δ2 T cell. In some embodiments, the therapeutic cell is a δ1 T cell. In some embodiments, the therapeutic cell is a δ3 T cell.

In some embodiments according to any one of the modified therapeutic cells or methods described above, the therapeutic cell is a stem cell. In some embodiments, the therapeutic cell is an embryonic stem cell. In some embodiments, the therapeutic cell is a hematopoictic stem cell (HSC).

In some embodiments according to any one of the modified therapeutic cells or methods described above, the therapeutic cell further comprises a heterologous nucleic acid sequence encoding an engineered receptor. In some embodiments, the engineered receptor is selected from the group consisting of a chimeric antigen receptor (CAR), a recombinant T cell receptor (TCR), a T-cell antigen coupler (TAC) receptor and a TCR fusion protein (TFP). In some embodiments, the therapeutic cell is a CAR-T cell. In some embodiments, the engineered receptor targets a tumor antigen selected from the group consisting of CD19, BCMA, NY-ESO-1, VEGFR2, MAGE-A3, CD20, CD22, CD33, CD38, CEA, EGFR, GD2, HER2, IGF1R, mesothelin, PSMA, ROR1, WT1 and combinations thereof. In some embodiments, the engineered receptor is an anti-BCMA CAR. In some embodiments, the engineered receptor is an anti-CD19 CAR.

In some embodiments according to any one of the modified therapeutic cells or methods described above, the Major Histocompatibility Complex (MHC) genes of the therapeutic cell are not genetically modified.

In some embodiments according to any one of the modified therapeutic cells or methods described above, the first heterologous nucleic acid sequence is operably linked to a promoter. In some embodiments, the first heterologous nucleic acid sequence is present in a vector (e.g., viral vector such as lentiviral vector).

In some embodiments according to any one of the modified therapeutic cells or methods described above, expression of the sICP47 protein or functional variant thereof downregulates cell surface expression of MHC molecules in the therapeutic cell. In some embodiments, expression of the sICP47 protein or functional variant thereof downregulates cell surface expression of MHC molecules in the therapeutic cell by at least about any one of 10%, 20%, 30%, 40%, 50%, 60%, 70%, 80%, 90% or more, compared to a therapeutic cell that does not express the sICP47 protein or functional variant thereof.

One aspect of the present application provides a method of producing a modified therapeutic cell, comprising introducing into a precursor cell a first heterologous nucleic acid sequence encoding a sICP47 or a functional variant thereof. In some embodiments, the method further comprises introducing into the precursor cell a second heterologous nucleic acid sequence encoding an agonist agent of an NK cell inhibitory receptor, such as a membrane-bound anti-NKG2A antibody. Also provided is a modified therapeutic cell obtained by any one of the methods of production described above.

Another aspect of the present application provides a method of down-regulating expression of MHC class I molecule on a modified therapeutic cell, comprising introducing into a precursor cell a first heterologous nucleic acid sequence encoding a sICP47 or a functional variant thereof. In some embodiments, the method further comprises introducing into the precursor cell a second heterologous nucleic acid sequence encoding an agonist agent of an NK cell inhibitory receptor, such as a membrane-bound anti-NKG2A antibody (mNKG2A).

One aspect of the present application provides a pharmaceutical composition comprising the modified therapeutic cell according to any one of the modified therapeutic cells described above.

One aspect of the present application provides a method of treating a disease or condition in an individual in need thereof, comprising administering to the individual an effective amount of the pharmaceutical composition according to any one of the pharmaceutical compositions described above.

In some embodiments according to any one of the methods of treatment described above, the modified therapeutic cell is allogeneic. In some embodiments, the individual is human.

In some embodiments according to any one of the methods of treatment described above, one or more human leukocyte antigen (HLA) alleles of the individual have mismatching allotypes compared to those of the modified therapeutic cell. In some embodiments, one or more (e.g., 8 out of 8, 7 out of 8, or 6 out of 8) alleles of HLA-A, HLA-B, HLA-C and HLA-DRB1 mismatching allotypes compared to those of the modified therapeutic cell. In some embodiments, all tested HLA alleles of the individual have matching allotypes compared to those of the modified therapeutic cell.

In some embodiments according to any one of the methods of treatment described above, the method reduces undesired immune response against the therapeutic cell in the individual compared to a method using a therapeutic cell that does not comprise the first heterologous sequence encoding the sICP47 protein or functional variant thereof. In some embodiments, the undesired immune response comprises Host-versus-Graft (HvG) response. In some embodiments, the HvG response is mediated by T cells of the individual. In some embodiments, the HvG response is mediated by NK cells of the individual.

In some embodiments according to any one of the methods of treatment described above, the method induces immune tolerance towards the therapeutic cell in the individual compared to a method using a therapeutic cell that does not comprise the first heterologous nucleic acid sequence encoding the sICP47 protein or functional variant thereof.

In some embodiments according to any one of the methods of treatment described above, the method increases persistence of the therapeutic cell in the individual compared to a method using a therapeutic cell that does not comprise the first heterologous nucleic acid sequence encoding the sICP47 protein or functional variant thereof.

In some embodiments according to any one of the methods described above, the disease or condition is a cancer, an infectious disease, or an autoimmune disease.

One aspect of the present application provides a kit comprising the modified therapeutic cell according to any one of the modified therapeutic cells described above and instructions for treating a disease or condition in an individual in need thereof. In some embodiments, the modified therapeutic cell is allogeneic. In some embodiments, the modified therapeutic cell is an allogeneic CAR-T cell. In some embodiments, the modified therapeutic cell is an allogeneic CAR-NK cell. In some embodiments, the modified therapeutic cell is an allogeneic CAR-NKT cell. In some embodiments, the modified therapeutic cell is an allogeneic CAR-γδ T cell.

Compositions, uses, kits and articles of manufacture comprising any one of the modified therapeutic cells described herein are also provided.

### BRIEF DESCRIPTION OF THE DRAWINGS

FIGS. 1A-1D show HLA-ABC expression on wild type T cells, sICP47 SA8-T cells, and B2M knockout T cells. By overexpressing sICP47 SA8 in T cells, or knocking out B2M on T cells, the HLA-ABC expression levels are down-regulated at different levels compared to the wild type T cells. When B2M was knockout on primary T, there was no detectable B2M on T cells' surface.

FIG. 2A shows anti-alloreaction effects of B2M knockout on T cells and sICP47 SA8-T cells when co-cultured with allogeneic wild type T cells. FIG. 2B shows anti-alloreaction effects of B2M knockout on T cells and sICP47 SA8-T cells when co-cultured with allogeneic CD8+ T cells. FIG. 2C shows anti-alloreaction effects of B2M knockout on T cells and sICP47 SA8-T cells when co-cultured with allogeneic NK cells. Using allogeneic wild type T cells or allogeneic CD8+ T cells to co-culture with the wild type T, B2M KO T or sICP47 expressed T cells, B2M KO T cells and sICP47 SA8-T cells showed anti-alloreaction effect as compared to wild type T cells.

FIG. 3A shows that membrane-bound anti-NKG2A antibody expressing B2M knockout T cells can partially inhibit the killing by allo-UnT (wildtype T) cells when compared with B2M knockout T cells. FIG. 3B shows that membrane-bound anti-NKG2A antibody expressing B2M knockout T cells can partially inhibit the killing by allo-CD8+ T cells when compared with B2M knockout T cells. Both B2M knockout T cells and membrane bound anti-NKG2A antibody expressing B2M knockout T cells can escape killing by allo-wild type T cells and allo-CD8+ T cells. FIG. 3C shows that membrane-bound anti-NKG2A antibody expressing B2M knockout T cells have improved resistance to killing by allo-NK cells compared to B2M knockout T cells.

FIG. 4A shows that sICP47 SA8+NKG2A antibody expressing T cells and B2M knockout T cells have different levels of down-regulation of HLA-ABC compared to the wild type T cells.

FIGS. 4B-4D show anti-alloreaction effects of wild type T cells, B2M knockout T cells and sICP47 SA8+mNKG2A (membrane-bound NKG2A) antibody expressing T cells when co-cultured with allogeneic wild type T cells (FIG. 4B), allogeneic CD8+ T cells (FIG. 4C) or allo-NK cells (FIG. 4D), respectively.

FIG. 5A shows in vivo anti-alloreaction effects of wild type T cells, B2M knockout T cells and sICP47 SA8+mNKG2A antibody expressing T cells against host PBMC in NCG mice.

FIG. 5B shows in vivo anti-alloreaction effects of wild type T cells, 132M knockout T cells and sICP47+mNKG2A antibody expressing T cells against host NK cells in NCG mice.

FIG. 6 shows sequence alignments of sICP47 SA8 (SEQ ID NO 4), sICP47 CeHV-16 (SEQ ID NO 5), sICP47 CeHV-1 (SEQ ID NO 6), ICP47 HHV11 (SEQ ID NO 19), and ICP47 HHV2H (SEQ ID NO 20).

FIG. 7 shows HLA-ABC expression on wild type T cells, sICP47 SA8-T cells, sICP47 CeHV-16-T cells, sICP47 CeHV-1-T cells, and ICP47 HHV2H-T cells and B2M knockout T cells.

FIGS. 8A-8B show anti-alloreaction effects of wild type T cells, B2M knockout T cells and sICP47 SA8-T cells, sICP47 CeHV-16-T cells, sICP47 CeHV-1-T cells, and ICP47 HHV2H-T cells when co-cultured with allogeneic wild type T cells (FIG. 8A), or allo-NK cells (FIG. 8B), respectively.

### DETAILED DESCRIPTION OF THE INVENTION

The present application provides modified therapeutic cells comprising a first heterologous nucleic acid encoding a simian ICP47 (sICP47) protein or a functional variant thereof. The modified therapeutic cells described herein are associated with reduced allo-reactive immune response mediated by both T cells and natural killer (NK) cells from the host. In some embodiments, the modified therapeutic cell further comprises a second heterologous nucleic acid sequence encoding an agonist of an NK cell inhibitory receptor. Methods of treating a disease or condition using the modified therapeutic cells and methods of reducing undesirable immune response in a patient receiving allogeneic therapeutic cells are also provided. The compositions and methods described herein are applicable to a wide range of adoptive cell therapy, including therapies using adaptive immunity cells such as αβ T cells, γδ T cells, and Natural Killer T (NKT) cells, innate immunity cells such as NK cells, stem cells such as hematopoictic stem cells (I-SC), as well as products modified with engineered receptors such as Chimeric Antigen Receptor (CAR) modified. The compositions and methods described herein reduce graft rejection mediated by both host T cells and NK cells, induce peripheral immune tolerance, increase in vivo persistence and increase efficacy of an adoptive cell therapy in a patient.

Without being bound by any theory or hypothesis, sICP47 expression may prevent assembly of Major Histocompatibility Complex (MHC) Class I molecules and alloantigen presentation on adoptively transferred CAR-T cells. Inventors of the present application observed reduced host T cell (including CD8+ T cell) recognition and activity against allogeneic CAR-T cells expressing sICP47. Furthermore, host NK-cell mediated allo-reaction was partially reduced against allogeneic CAR-T cells expressing sICP47. Additive inhibitory effects on host NK-cell response could be achieved by combining sICP47 expression with expression of a membrane-bound anti-NKG2A binding domain in allogeneic CAR-T cells.

Accordingly, one aspect of the present application provides a modified therapeutic cell comprising a first heterologous nucleic acid sequence encoding a sICP47 protein or a functional variant thereof. In some embodiments, the modified therapeutic cell further comprises a second nucleic acid sequence encoding an agonist of an NK cell inhibitory receptor such as an NKG2A receptor.

One aspect of the present application provides a method of reducing graft rejection of allogeneic therapeutic cells in an individual in need thereof, comprising administering to the individual an effective amount of the allogeneic therapeutic cells, wherein the allogeneic therapeutic cells comprise a first heterologous nucleic acid sequence encoding a first heterologous nucleic acid sequence encoding a sICP47 protein or a functional variant thereof. In some embodiments, the allogeneic therapeutic cells further comprise a second nucleic acid sequence encoding an agonist of an NK cell inhibitory receptor such as an NKG2A receptor.

### I. Definitions

As used herein, “simian ICP47” and “sICP47” are used interchangeably to refer to an ICP47 protein of a herpesvirus whose natural host is a non-human primate. In some embodiments, the sICP47 is an ICP47 protein of Simian Agent 8 (SA), a type 2 Herpes simplex virus (HSV) that infects Cercopithecine monkeys such as baboons. In some embodiments, the sICP47 is an ICP47 protein of Cercopithecine herpesvirus 16 (CeHV-16). In some embodiments, the sICP47 is an ICP47 protein of Cercopithecine herpesvirus 1 (e.g., strain E2490).

“Functional variants” of the sICP47 proteins described herein comprise full-length or a portion of a naturally occurring sICP47, generally including the active domain, which retains ability to bind to transporters associated with antigen processing (TAP) and to inhibit assembly of MHC class I complexes. In some embodiments, a functional variant of a sICP47 protein is at least about 25 amino acid residues long, such as at least about any one of 30, 33, 35, 38, 40, 45, 50, 55, 60, 65, 70 or more amino acid residues long. “Functional variants” also include polypeptides having at least about 80% sequence identity (e.g., at least about any one of 85%, 90%, 91%, 92%, 93%, 94%, 95%, 96%, 97%, 98%, 99% or more) to a naturally occurring sICP47 or a portion thereof.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of the disease. The methods of the present application contemplate any one or more of these aspects of treatment.

The term “prevent,” and similar words such as “prevented,” “preventing” etc., indicate an approach for preventing, inhibiting, or reducing the likelihood of the recurrence of, a disease or condition, e.g., cancer. It also refers to delaying the recurrence of a disease or condition or delaying the recurrence of the symptoms of a disease or condition. As used herein, “prevention” and similar words also includes reducing the intensity, effect, symptoms and/or burden of a disease or condition prior to recurrence of the disease or condition.

As used herein, “delaying” the development of a disease means to defer, hinder, slow, retard, stabilize, and/or postpone development of the disease. This delay can be of varying lengths of time, depending on the history of the disease and/or individual being treated. A method that “delays” development of a disease is a method that reduces probability of disease development in a given time frame and/or reduces the extent of the disease in a given time frame, when compared to not using the method. Such comparisons are typically based on clinical studies, using a statistically significant number of individuals. Disease development can be detectable using standard methods, including, but not limited to, computerized axial tomography (CAT Scan), Magnetic Resonance Imaging (MRI), abdominal ultrasound, clotting tests, arteriography, or biopsy. Development may also refer to disease progression that may be initially undetectable and includes occurrence, recurrence, and onset.

The term “effective amount” used herein refers to an amount of an agent sufficient to treat a specified disorder, condition or disease such as ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay recurrence. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

As used herein, an “individual” or a “subject” refers to a mammal, including, but not limited to, human, bovine, horse, feline, canine, rodent, or primate. In some embodiments, the individual is a human.

The term “antibody” includes monoclonal antibodies, antibody compositions with polyepitopic specificity, multispecific antibodies (e.g., bispecific antibodies, diabodies, and single-chain molecules), as well as antibody fragments (e.g., Fab, F(ab′)₂, and Fv). The term “immunoglobulin” (Ig) is used interchangeably with “antibody” herein. The term antibody includes conventional four-chain antibodies, and single-domain antibodies, such as heavy-chain only antibodies or fragments thereof, e.g., V_{H}H.

The term “heavy chain-only antibody” or “HCAb” refers to a functional antibody, which comprises heavy chains, but lacks the light chains usually found in 4-chain antibodies. Camelid animals (such as camels, llamas, or alpacas) are known to produce HCAbs.

The term “single-domain antibody” or “sdAb” refers to a single antigen-binding polypeptide having three complementary determining regions (CDRs). The sdAb alone is capable of binding to the antigen without pairing with a corresponding CDR-containing polypeptide. In some cases, single-domain antibodies are engineered from camelid HCAbs, and their heavy chain variable domains are referred herein as “V_{H}Hs” (Variable domain of the heavy chain of the Heavy chain antibody). Camelid sdAb is one of the smallest known antigen-binding antibody fragments (see, e.g., Hamers-Castennan et al., Nature 363:446-8 (1993); Greenberg et al., Nature 374:168-73 (1995); Hassanzadeh-Ghassabeh et al., Nanomedicine (Lond), 8:1013-26 (2013)). A basic V_{H}H has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

The term “constant domain” refers to the portion of an immunoglobulin molecule having a more conserved amino acid sequence relative to the other portion of the immunoglobulin, the variable domain, which contains the antigen-binding site. The constant domain contains the C_{H}1, C_{H}2 and C_{H}3 domains (collectively, CH) of the heavy chain and the CHL (or CL) domain of the light chain.

The “variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites. Heavy-chain only antibodies from the Camelid species have a single heavy chain variable region, which is referred to as “V_{H}H”. V_{H}H is thus a special type of V_{H}.

The “light chains” of antibodies (immunoglobulins) from any mammalian species can be assigned to one of two clearly distinct types, called kappa (“K”) and lambda (“V”), based on the amino acid sequences of their constant domains.

“Antibody fragments” comprise a portion of an intact antibody, preferably comprising the antigen-binding region thereof. In some embodiments, the antibody fragment described herein is an antigen-binding fragment. Examples of antibody fragments include, but are not limited to Fab, Fab′, F(ab′)₂ and Fv fragments; diabodies; linear antibodies (see U.S. Pat. No. 5,641,870, Example 2; Zapata et al., Protein Eng. 8(10): 1057-1062 (1995)); single-chain antibody (scFv) molecules; single-domain antibodies (such as V_{H}H), and multispecific antibodies formed from antibody fragments. Papain digestion of antibodies produced two identical antigen-binding fragments, called “Fab” fragments, and a residual “Fe” fragment, a designation reflecting the ability to crystallize readily. The Fab fragment consists of an entire L chain along with the variable domain of the H chain (Vii), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment that roughly corresponds to two disulfide linked Fab fragments having different antigen-binding activity and is still capable of cross-linking antigen. Fab′ fragments differ from Fab fragments by having a few additional residues at the carboxy-terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments, which have hinge cysteines between them. Other chemical couplings of antibody fragments are also known.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains, which enables the scFv to form the desired structure for antigen binding. For a review of scFv, see, e.g., Pluckthin, The Pharmacology of Monoclonal Antibodies. Springer Berlin Heidelberg, 1994. 269-315.

The term “hypervariable region,” “HVR,” or “HV,” when used herein refers to the regions of an antibody variable domain, which are hypervariable in sequence, and/or form structurally defined loops. Generally, single-domain antibodies comprise three HVRs (or CDRs): HVR1 (or CDR1), HVR2 (or CDR2), and HVR3 (or CDR3). HVR3 (or CDR3) displays the most diversity of the three HVRs, and is believed to play a unique role in conferring fine specificity to antibodies. See, e.g., Hamers-Casterman et al., Nature 363:446-448 (1993); Sheriff et al., Nature Struct. Biol. 3:733-736 (1996).

The term “Complementarity Determining Region” or “CDR” are used to refer to hypervariable regions as defined by the Kabat system. See Kabat et al., Sequences of Proteins of Immunological Interest, 5th Ed. Public Health Service, National Institutes of Health, Bethesda, Md. (1991)

A number of HVR delineations are in use and are encompassed herein. The Kabat Complementarity Determining Regions (CDRs) are based on sequence variability and are the most commonly used. (Kabat et al., Sequences of Proteins of Immunological Interest, 5th Ed. Public Health Service, National institutes of Health, Bethesda, Md. (1991)). Chothia refers instead to the location of the structural loops (Chothia and Lesk, J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below in Table 1.

TABLE 1
HVR delineations.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | (Kabat Numbering) | | |
| H1 | H31-H35 | H26-H35 | H26-H32 | H30-H35 |
| | | (Chothia Numbering) | | |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the V_{L} and 26-35 (H1), 50-65 or 49-65 (H2) and 93-102, 94-102, or 95-102 (H3) in the V_{H}. The variable domain residues are numbered according to Kabat et al., supra, for each of these definitions.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

Unless indicated otherwise herein, the numbering of the residues in an immunoglobulin heavy chain is that of the EU index as in Kabat et al, supra. The “EU index as in Kabat” refers to the residue numbering of the human IgG1 EU antibody.

“Framework” or “FR” residues are those variable-domain residues other than the HVR residues as herein defined.

The term “Fc region” or “fragment crystallizable region” herein is used to define a C-terminal region of an immunoglobulin heavy chain, including native-sequence Fe regions and variant Fe regions. Although the boundaries of the Fe region of an immunoglobulin heavy chain might vary, the human IgG heavy-chain Fe region is usually defined to stretch from an amino acid residue at position Cys226, or from Pro230, to the carboxyl-terminus thereof. The C-terminal lysine (residue 447 according to the EU numbering system) of the Fe region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fe regions for use in the antibodies described herein include human IgG1, IgG2 (IgG2A, IgG2B), IgG3 and IgG4.

As use herein, the term “binds”, “specifically binds to” or is “specific for” refers to measurable and reproducible interactions such as binding between a target and a ligand, which is determinative of the presence of the target in the presence of a heterogeneous population of molecules including biological molecules. For example, a ligand that binds to or specifically binds to a target is a ligand that binds this target with greater affinity, avidity, more readily, and/or with greater duration than it binds to other targets. In one embodiment, the extent of binding of a ligand to an unrelated target is less than about 10% of the binding of the ligand to the target as measured, e.g., by a radioimmunoassay (RIA). In certain embodiments, a ligand that specifically binds to a target has a dissociation constant (Kd) of ≤1 μM, ≤100 nM, ≤10 nM, ≤1 nM, or ≤0.1 nM. In certain embodiments, a ligand specifically binds to a region on a protein that is conserved among the proteins from different species. In another embodiment, specific binding can include, but does not require exclusive binding.

“Chimeric antigen receptor” or “CAR” as used herein refers to engineered receptors, including genetically engineered receptors, which can be used to graft one or more antigen specificity onto immune effector cells, such as T cells. Some CARs are also known as “artificial T-cell receptors,” “chimeric T cell receptors,” or “chimeric immune receptors.” An exemplary chimeric antigen receptor may include one or more antigen-binding portions (such as a single domain antibody or scFv) and a signaling domain, such as a signaling domain from a T cell receptor (e.g., CD3ζ). In some embodiments, a CAR comprises an antigen-binding moiety, a transmembrane domain and an intracellular domain. The intracellular domain may include a signaling chain having an immunoreceptor tyrosine-based activation motif (ITAM), such as CD3ζ or FcεRIγ. In some embodiments, the intracellular domain further comprises the intracellular portion of at least one co-stimulatory domain, such as CD28, 4-1BB (CD137), ICOS, OX40 (CD134), CD27, hematopoietic cell signal transducer (DAP10) and/or Killer-activating receptor-associated protein (DAP12). In the context of the present application, the terms “cytoplasmic domain”, “intracellular domain” and “intracellular signaling domain” are interchangeable. In some embodiments, the CAR comprises an extracellular antigen binding domain specific for one or more antigens (such as tumor antigens), a transmembrane domain, and an intracellular signaling domain of a T cell receptor and/or other receptors. CARs also include single CARs, dual CARs, tandem CARs and split CARs. In some embodiments, the CAR is a single CAR. In some embodiments, the CAR is a dual CAR. In some embodiments, the CAR is a tandem CAR. In some embodiments, the CAR is a split CAR.

“CAR-T cell” refers to a T cell that expresses a CAR. The term “single CAR” as used herein refers to a chimeric molecule that comprises a single antigen-binding portion (such as a single domain antibody or scFv) and a signaling domain, such as a signaling domain from a T cell receptor (e.g., CD3ζ). In some embodiments, a single CAR comprises a monospecific antigen-binding moiety, a transmembrane domain, and an intracellular domain. In some embodiments, a single CAR comprises an extracellular antigen binding domain specific for one or more antigens (such as tumor antigens), a transmembrane domain, and an intracellular signaling domain of a T cell receptor and/or other receptors.

“UCAR-T” or “universal CAR-T cells” refers to off-the-shelf CAR-modified T cells that can be used to treat an allogeneic patient in need thereof. UCAR-Ts include those that contain genetic modification in addition to the CAR construct, and those that do not contain genetic modification other than the CAR construct.

“T cell receptor” or “TCR” as used herein refers to endogenous or recombinant T cell receptor comprising an extracellular antigen-binding domain that binds to a specific antigenic peptide bound in an MHC molecule. In some embodiments, the TCR comprises a TCRα polypeptide chain and a TCRβ polypeptide chain. In some embodiments, the TCR specifically binds a tumor antigen. “TCR-T” refers to a T cell that expresses a recombinant TCR.

“T-cell antigen coupler receptor” or “TAC receptor” as used herein refers to an engineered receptor comprising an extracellular antigen-binding domain that binds to a specific antigen and a T-cell receptor (TCR) binding domain, a transmembrane domain, and an intracellular domain of a co-receptor molecule. The TAC receptor co-opts the endogenous TCR of a T cell that expressed the TAC receptor to elicit antigen-specific T-cell response against a target cell.

“TCR fusion protein” or “TFP” as used herein refers to an engineered receptor comprising an extracellular antigen-binding domain that binds to a specific antigen fused to a subunit of the TCR complex or a portion thereof, including TCRα chain, TCRβ chain, TCRγ chain, TCRδ chain, CD3ε, CD3δ, or CD3γ. The subunit of the TCR complex or portion thereof comprise a transmembrane domain and at least a portion of the intracellular domain of the naturally occurring TCR subunit. In some embodiments, the TFP comprises the extracellular domain of the TCR subunit or a portion thereof. In some embodiments, the TFP does not comprise the extracellular domain of the TCR subunit.

“Percent (%) amino acid sequence identity” and “homology” with respect to a polypeptide sequence are defined as the percentage of amino acid residues in a candidate sequence that are identical with the amino acid residues in the specific polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared. For example, polypeptides having at least 70%, 85%, 90%, 95%, 98% or 99% identity to specific polypeptides described herein and preferably exhibiting substantially the same functions, as well as polynucleotide encoding such polypeptides, are contemplated.

As used herein, the term “express” refers to transcription of a DNA to an RNA (e.g., mRNA), or translation of a nucleic acid into a protein. Proteins may be expressed and remain intracellular, become a component of the cell surface membrane, or be secreted into extracellular matrix or medium.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is a cell, which has been transfected, transformed or transduced with exogenous nucleic acid.

As used herein, the expressions “cell”, “cell line”, and “cell culture” are used interchangeably and all such designations include progeny. Thus, the words “transfectants” and “transfected cells” include the primary subject cell and cultures derived there from without regard for the number of transfers. It is also understood that not all progeny may be precisely identical in DNA content, due to deliberate or inadvertent mutations. Variant progeny that have the same function or biological activity as screened for in the originally transformed cell are included.

“Primary cells” refer to cells taken directly from living tissue (i.e. biopsy material) and established for growth in vitro, which have undergone very few population doublings and are therefore more representative of the main functional components and characteristics of tissues from which they are derived from, in comparison to continuous tumorigenic or artificially immortalized cell lines.

The term “in vivo” refers to inside the body of the organism from which the cell is obtained. “Ex vivo” or “in vitro” means outside the body of the organism from which the cell is obtained.

As used herein, the term “autologous” refers to any material derived from the same individual to whom it is later to be re-introduced into the individual.

“Allogeneic” refers to a graft derived from a different individual of the same species.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

It is understood that embodiments of the present application described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a”, “an”, and “the” include plural referents unless the context clearly dictates otherwise.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Modified Therapeutic Cells

The present application provides a modified therapeutic cell (e.g., allogeneic immune cell or stem cell) associated with reduced undesired immune response, such as reduced graft rejection or Host-versus-Graft Disease (HvGD) including both T cell-mediated and NK cell-mediated HvGD, increased immune tolerance, and/or increased therapeutic efficacy. The modified therapeutic cells described herein comprise heterologous nucleic acid sequence(s) encoding a simian ICP47 (sICP47) protein or a functional variant thereof, and/or an agonist of a natural killer (NK) cell inhibitory receptor.

In some embodiments, there is provided a modified therapeutic cell comprising a first heterologous nucleic acid sequence encoding a sICP47 protein or a functional variant thereof. In some embodiments, the first heterologous nucleic acid sequence encodes a naturally occurring sICP47 protein (i.e., a full-length naturally occurring sICP47 protein). In some embodiments, the first heterologous nucleic acid sequence encodes an engineered sICP47 protein comprising an active domain of a naturally occurring sICP47 protein.

In some embodiments, there is provided a modified therapeutic cell comprising a first heterologous nucleic acid sequence encoding an active domain of a naturally occurring sICP47 protein or a functional variant thereof.

The sICP47 protein may be derived from any ICP47 protein of a herpesvirus that naturally infects monkeys. Exemplary sICP47 proteins are described in the section “A. Simian ICP47” below. In some embodiments, the naturally occurring sICP47 protein is from a herpesvirus selected from the group consisting of Simian Agent 8 (SA8) Cercopithecine herpesvirus 16 (CeHV-16), Cercopithecine herpesvirus 1 (CeHV-1), Macacine alphaherpesvirus 1, and Pappine alphaherpesvirus 2.

In some embodiments, there is provided a modified therapeutic cell comprising a first heterologous nucleic acid sequence encoding a sICP47 protein of SA8 or a functional variant thereof. In some embodiments, the first heterologous nucleic acid sequence encodes a full-length sICP47 of SA8. In some embodiments, the first heterologous nucleic acid sequence encodes a polypeptide comprising an active domain of a sICP47 protein of SA8.

In some embodiments, there is provided a modified therapeutic cell comprising a first heterologous nucleic acid sequence encoding a sICP47 protein of CeHV-16 or a functional variant thereof. In some embodiments, the first heterologous nucleic acid sequence encodes a full-length sICP47 of CeHV-16. In some embodiments, the first heterologous nucleic acid sequence encodes a polypeptide comprising an active domain of a sICP47 protein of CeHV-16.

In some embodiments, there is provided a modified therapeutic cell comprising a first heterologous nucleic acid sequence encoding a polypeptide comprising an amino acid sequence having at least about 80% (e.g., at least about any one of 85%, 90%, 92%, 95%, 98%, 99%, or more) sequence identity to an amino acid sequence selected from the group consisting of SEQ ID NOs: 1-3.

In some embodiments, there is provided a modified therapeutic cell comprising a first heterologous nucleic acid sequence encoding a polypeptide comprising an amino acid sequence having at least about 80% (e.g., at least about any one of 85%, 90%, 92%, 95%, 98%, 99%, or more) sequence identity to an amino acid sequence selected from the group consisting of SEQ ID NOs: 4-6 and 24-25.

In some embodiments, there is provided a modified therapeutic cell comprising a first heterologous nucleic acid sequence encoding a polypeptide comprising the amino acid sequence of SEQ ID NO: 1. In some embodiments, there is provided a modified therapeutic cell comprising a first heterologous nucleic acid sequence encoding a polypeptide comprising the amino acid sequence of SEQ ID NO: 4.

In some embodiments, there is provided a modified therapeutic cell comprising a first heterologous nucleic acid sequence encoding a polypeptide comprising the amino acid sequence of SEQ ID NO: 2. In some embodiments, there is provided a modified therapeutic cell comprising a first heterologous nucleic acid sequence encoding a polypeptide comprising the amino acid sequence of SEQ ID NO: 5.

In some embodiments, there is provided a modified therapeutic cell comprising a first heterologous nucleic acid sequence encoding a polypeptide comprising the amino acid sequence of SEQ ID NO: 3. In some embodiments, there is provided a modified therapeutic cell comprising a first heterologous nucleic acid sequence encoding a polypeptide comprising the amino acid sequence of SEQ ID NO: 6.

In some embodiments, the modified therapeutic cell further comprises a second heterologous nucleic acid sequence encoding an agonist agent of a natural killer (NK) cell inhibitory receptor. Exemplary NK cell inhibitory receptors include, but are not limited to, killer cell immunoglobulin-like receptors (KIR), CD94-NKG2A/C/E heterodimers, 2B4 (CD244) receptor and Killer cell lectin-like receptor G1 (KLRG1). In some embodiments, the NK cell inhibitory receptor is an NKG2A receptor.

The agonist agent may be of any suitable molecular modality that specifically binds to a NK cell inhibitory receptor and activates the receptor. In some embodiments, the agonist agent is an agonist antibody or an antigen binding fragment thereof, such as an scFv, a single domain antibody (e.g., V_{H}H) or a Fab. In some embodiments, the agonist agent is a membrane-bound antibody. For example, the membrane-bound antibody may comprise an antigen binding domain fused to a transmembrane domain or a membrane anchor (e.g., a GPI anchor). In some embodiments, the membrane-bound antibody comprises a polypeptide comprising from the N-terminus to the C-terminus a single-chain variable fragment (scFv) or an sdAb (e.g., V_{H}H) that specifically binds to a NK cell inhibitory receptor, a CD8α hinge sequence and a CD8α transmembrane sequence. In some embodiments, the agonist antibody or antigen binding fragment thereof specifically binds to NKG2A. Suitable NKG2A agonist antibodies are known in the art, for example, Z270 and derivatives thereof as described in US20120237510A1, which is incorporated herein by reference in its entirety. In some embodiments, the agonist antibody is a human, humanized, or chimeric antibody.

In some embodiments, the agonist agent comprises one, two, three, four, five, or six CDRs of an anti-NKG2A antibody comprising a VH comprising the amino acid sequence of SEQ ID NO: 13 and a VL comprising the amino acid sequence of SEQ ID NO: 14. In some embodiments, the agonist agent comprises: (i) a heavy chain variable region (VH) comprising a heavy chain complementary determining region (HC-CDR) 1 comprising the amino acid sequence of SEQ ID NO: 7, an HC-CDR2 comprising the amino acid sequence of SEQ ID NO: 8, and an HC-CDR3 comprising the amino acid sequence of SEQ ID NO: 9; and (ii) a light chain variable region (VL) comprising a light chain complementary determining region (LC-CDR) 1 comprising the amino acid sequence of SEQ ID NO: 10, an LC-CDR2 comprising the amino acid sequence of SEQ ID NO: 11, and an LC-CDR3 comprising the amino acid sequence of SEQ ID NO: 12. In some embodiments, the agonist agent comprises a VH comprising HC-CDR1, HC-CDR2 and HC-CDR3 of SEQ ID NO: 13, and a VL comprising LC-CDR1, LC-CDR2 and LC-CDR3 of SEQ ID NO: 14. In some embodiments, the agonist agent comprises a VH comprising an amino acid sequence having at least about 80% (e.g., at least about any one of 85%, 90%, 92%, 95%, 98%, 99%, or more) sequence identity to SEQ ID NO: 13 and/or a VL comprising an amino acid sequence having at least about 80% (e.g., at least about any one of 85%, 90%, 92%, 95%, 98%, 99%, or more) sequence identity to SEQ ID NO: 14. In some embodiments, the agonist agent comprises a VH comprising the amino acid sequence of SEQ ID NO: 13 and a VL comprising the amino acid sequence of SEQ ID NO: 14.

In some embodiments, the agonist agent is a polypeptide comprising from the N-terminus to the C-terminus an anti-NKG2A single-chain variable fragment (scFv), a CD8α hinge sequence and a CD8α transmembrane sequence. In some embodiments, the agonist agent comprises an amino acid sequence having at least about 80% (e.g., at least about any one of 85%, 90%, 92%, 95%, 98%, 99%, or more) sequence identity to SEQ ID NO: 15. In some embodiments, the agonist agent comprises an amino acid sequence having at least about 80% (e.g., at least about any one of 85%, 90%, 92%, 95%, 98%, 99%, or more) sequence identity to SEQ ID NO: 16.

Also provided is a polypeptide comprising the amino acid sequence of SEQ ID NO: 16.

In some embodiments, the agonist agent is a ligand of an NK cell inhibitory receptor. In some embodiments, the agonist agent is a ligand of NKG2A, such as HLA-E or a derivative thereof.

In some embodiments, there is provided a modified therapeutic cell comprising a first heterologous nucleic acid sequence encoding a sICP47 protein or a functional variant thereof and a second heterologous nucleic acid sequence encoding an agonist agent of an NK cell inhibitory receptor (e.g., NKG2A receptor). In some embodiments, the first heterologous nucleic acid sequence encodes a naturally occurring sICP47 protein. In some embodiments, the first heterologous nucleic acid sequence encodes an engineered sICP47 protein comprising an active domain of a naturally occurring sICP47 protein. In some embodiments, the naturally occurring sICP47 protein is from a herpesvirus selected from the group consisting of Simian Agent 8 (SA8), Cercopithecine herpesvirus 16 (CeHV-16) and Cercopithecine herpesvirus 1 (CeHV-1). In some embodiments, the sICP47 protein or functional variant thereof comprises an amino acid sequence having at least about 80% (e.g., at least about any one of 85%, 90%, 92%, 95%, 98%, 99%, or more) sequence identity to an amino acid sequence selected from the group consisting of SEQ ID NOs: 1-3. In some embodiments, the sICP47 protein or functional variant thereof comprises an amino acid sequence having at least about 80% (e.g., at least about any one of 85%, 90%, 92%, 95%, 98%, 99%, or more) sequence identity to an amino acid sequence selected from the group consisting of SEQ ID NOs: 4-6 and 24-25. In some embodiments, the agonist agent is an antibody or antigen-binding fragment thereof that specifically binds to the NK cell inhibitory receptor. In some embodiments, the agonist agent is a membrane-bound antibody that specifically binds NKG2A. In some embodiments, the agonist agent is a ligand of the NK cell inhibitory receptor.

In some embodiments, there is provided a first heterologous nucleic acid sequence encoding a sICP47 protein of SA8 or a functional variant thereof and a second heterologous nucleic acid sequence encoding an agonist agent of an NK cell inhibitory receptor (e.g., NKG2A receptor). In some embodiments, the first heterologous nucleic acid sequence encodes a full-length sICP47 of SA8. In some embodiments, the first heterologous nucleic acid sequence encodes a polypeptide comprising an active domain of a sICP47 protein of SA8. In some embodiments, the sICP47 protein or functional variant thereof comprises an amino acid sequence having at least about 80% (e.g., at least about any one of 85%, 90%, 92%, 95%, 98%, 99%, or more) sequence identity to SEQ ID NO: 1. In some embodiments, the sICP47 protein or functional variant thereof comprises an amino acid sequence having at least about 80% (e.g., at least about any one of 85%, 90%, 92%, 95%, 98%, 99%, or more) sequence identity to SEQ ID NO: 4. In some embodiments, the agonist agent is an antibody or antigen-binding fragment thereof that specifically binds to the NK cell inhibitory receptor. In some embodiments, the agonist agent is a membrane-bound antibody that specifically binds NKG2A. In some embodiments, the agonist agent is a ligand of the NK cell inhibitory receptor.

In some embodiments, there is provided a first heterologous nucleic acid sequence encoding a sICP47 protein of SA8 or a functional variant thereof and a second heterologous nucleic acid sequence encoding an anti-NKG2A antibody comprising a VH comprising a HC-CDR1 comprising SEQ ID NO: 7, a HC-CDR2 comprising SEQ ID NO: 8, a HC-CDR3 comprising SEQ ID NO: 9, and a VL comprising a LC-CDR1 comprising SEQ ID NO: 10, a LC-CDR2 comprising SEQ ID NO: 11, and a LC-CDR3 comprising SEQ ID NO: 12. In some embodiments, the agonist agent comprises a VH comprising SEQ ID NO: 13 and a VL comprising SEQ ID NO: 14. In some embodiments, the anti-NKG2A antibody is a membrane-bound antibody. In some embodiments, the anti-NKG2A antibody comprises a polypeptide comprising from the N-terminus to the C-terminus a single-chain variable fragment (scFv) that specifically binds to NKG2A, a CD8α hinge sequence and a CD8α transmembrane sequence. In some embodiments, the first heterologous nucleic acid sequence encodes a full-length sICP47 of SA8. In some embodiments, the first heterologous nucleic acid sequence encodes a polypeptide comprising an active domain of a sICP47 protein of SA8. In some embodiments, the sICP47 protein or functional variant thereof comprises an amino acid sequence having at least about 80% (e.g., at least about any one of 85%, 90%, 92%, 95%, 98%, 99%, or more) sequence identity to SEQ ID NO: 1. In some embodiments, the sICP47 protein or functional variant thereof comprises an amino acid sequence having at least about 80% (e.g., at least about any one of 85%, 90%, 92%, 95%, 98%, 99%, or more) sequence identity to SEQ ID NO: 4.

In some embodiments, there is provided a first heterologous nucleic acid sequence encoding a sICP47 protein of CeHV-16 or a functional variant thereof and a second heterologous nucleic acid sequence encoding an agonist agent of an NK cell inhibitory receptor (e.g., NKG2A receptor). In some embodiments, the first heterologous nucleic acid sequence encodes a full-length sICP47 of CeHV-16. In some embodiments, the first heterologous nucleic acid sequence encodes a polypeptide comprising an active domain of a sICP47 protein of CeHV-16. In some embodiments, the sICP47 protein or functional variant thereof comprises an amino acid sequence having at least about 80% (e.g., at least about any one of 85%, 90%, 92%, 95%, 98%, 99%, or more) sequence identity to SEQ ID NO: 2. In some embodiments, the sICP47 protein or functional variant thereof comprises an amino acid sequence having at least about 80% (e.g., at least about any one of 85%, 90%, 92%, 95/a, 98%, 99%, or more) sequence identity to SEQ ID NO: 5. In some embodiments, the agonist agent is an antibody or antigen-binding fragment thereof that specifically binds to the NK cell inhibitory receptor. In some embodiments, the agonist agent is a membrane-bound antibody that specifically binds NKG2A. In some embodiments, the agonist agent is a ligand of the NK cell inhibitory receptor.

In some embodiments, there is provided a first heterologous nucleic acid sequence encoding a sICP47 protein of CeHV-16 or a functional variant thereof and a second heterologous nucleic acid sequence encoding an anti-NKG2A antibody comprising a VH comprising a HC-CDR1 comprising SEQ ID NO: 7, a HC-CDR2 comprising SEQ ID NO: 8, a HC-CDR3 comprising SEQ ID NO: 9, and a VL comprising a LC-CDR1 comprising SEQ ID NO: 10, a LC-CDR2 comprising SEQ ID NO: 11, and a LC-CDR3 comprising SEQ ID NO: 12. In some embodiments, the agonist agent comprises a VH comprising SEQ ID NO: 13 and a VL comprising SEQ ID NO: 14. In some embodiments, the anti-NKG2A antibody is a membrane-bound antibody. In some embodiments, the anti-NKG2A antibody comprises a polypeptide comprising from the N-terminus to the C-terminus a single-chain variable fragment (scFv) that specifically binds to NKG2A, a CD8α hinge sequence and a CD8α transmembrane sequence. In some embodiments, the first heterologous nucleic acid sequence encodes a full-length sICP47 of CEHV-16. In some embodiments, the first heterologous nucleic acid sequence encodes a polypeptide comprising an active domain of a sICP47 protein of CeHV-16. In some embodiments, the sICP47 protein or functional variant thereof comprises an amino acid sequence having at least about 80% (e.g., at least about any one of 85%, 90%, 92%, 95%, 98%, 99%, or more) sequence identity to SEQ ID NO: 2. In some embodiments, the sICP47 protein or functional variant thereof comprises an amino acid sequence having at least about 80% (e.g., at least about any one of 85%, 90%, 92%, 95%, 98%, 99%, or more) sequence identity to SEQ ID NO: 5.

In some embodiments, there is provided a first heterologous nucleic acid sequence encoding a sICP47 protein of CeHV-1 or a functional variant thereof and a second heterologous nucleic acid sequence encoding an agonist agent of an NK cell inhibitory receptor (e.g., NKG2A receptor). In some embodiments, the first heterologous nucleic acid sequence encodes a full-length sICP47 of CeHV-1. In some embodiments, the first heterologous nucleic acid sequence encodes a polypeptide comprising an active domain of a sICP47 protein of CeHV-1. In some embodiments, the sICP47 protein or functional variant thereof comprises an amino acid sequence having at least about 80% (e.g., at least about any one of 85%, 90%, 92%, 95%, 98%, 99%, or more) sequence identity to SEQ ID NO: 3. In some embodiments, the sICP47 protein or functional variant thereof comprises an amino acid sequence having at least about 80% (e.g., at least about any one of 85%, 90%, 92%, 95%, 98%, 99%, or more) sequence identity to SEQ ID NO: 6. In some embodiments, the agonist agent is an antibody or antigen-binding fragment thereof that specifically binds to the NK cell inhibitory receptor. In some embodiments, the agonist agent is a membrane-bound antibody that specifically binds NKG2A. In some embodiments, the agonist agent is a ligand of the NK cell inhibitory receptor.

In some embodiments, there is provided a first heterologous nucleic acid sequence encoding a sICP47 protein of CeHV-1 or a functional variant thereof and a second heterologous nucleic acid sequence encoding an anti-NKG2A antibody comprising a VH comprising a HC-CDR1 comprising SEQ ID NO: 7, a HC-CDR2 comprising SEQ ID NO: 8, a HC-CDR3 comprising SEQ ID NO: 9, and a VL comprising a LC-CDR1 comprising SEQ ID NO: 10, a LC-CDR2 comprising SEQ ID NO: 11, and a LC-CDR3 comprising SEQ ID NO: 12. In some embodiments, the agonist agent comprises a VH comprising SEQ ID NO: 13 and a VL comprising SEQ ID NO: 14. In some embodiments, the anti-NKG2A antibody is a membrane-bound antibody. In some embodiments, the anti-NKG2A antibody comprises a polypeptide comprising from the N-terminus to the C-terminus a single-chain variable fragment (scFv) that specifically binds to NKG2A, a CD8α hinge sequence and a CD8α transmembrane sequence. In some embodiments, the first heterologous nucleic acid sequence encodes a full-length sICP47 of CEHV-1. In some embodiments, the first heterologous nucleic acid sequence encodes a polypeptide comprising an active domain of a sICP47 protein of CeHV-1. In some embodiments, the sICP47 protein or functional variant thereof comprises an amino acid sequence having at least about 80% (e.g., at least about any one of 85%, 90%, 92%, 95%, 98%, 99%, or more) sequence identity to SEQ ID NO: 3. In some embodiments, the sICP47 protein or functional variant thereof comprises an amino acid sequence having at least about 80% (e.g., at least about any one of 85%, 90%, 92%, 95%, 98%, 99%, or more) sequence identity to SEQ ID NO: 6.

The modified therapeutic cells can be derived from a variety of cell types and cell sources. Cells from any mammalian species, including, but not limited to, mice, rats, guinea pigs, rabbits, dogs, monkeys, and humans, are contemplated herein. In some embodiments, the therapeutic cell is a human cell.

The modified therapeutic cells may be used as adoptive cell therapies to treat a disease or condition of the individual, such as cancer, autoimmune disease, or infectious disease. Exemplary adoptive cell therapies, include, but are not limited to, tumor infiltrating lymphocytes (TIL), T cell receptor (TCR) modified T cells (TCR-Ts), chimeric antigen receptor (CAR) modified T cells, natural killer (NK) cells, NKT cells, and hematopoietic stem cells (HSCs), and dendritic cell (DC) or myeloid cell therapy. In some embodiments, the modified therapeutic cell is an αβ T cell. In some embodiments, the modified therapeutic cell is a γδ T cells. In some embodiments, the modified therapeutic cell is a γ9δ2 T cell. In some embodiments, the modified therapeutic cell is a δ1 T cell. In some embodiments, the modified therapeutic cell is a δ3 T cell.

In some embodiments, the therapeutic cell is autologous, i.e., the cell is derived from the individual who receives the therapeutic cells. In some embodiments, the therapeutic cell is syngeneic (i.e., the donor and the recipients are different individuals, but are identical twins, triplets or quadruplets, etc.). In some embodiments, the therapeutic cell is allogeneic, i.e., the cell is obtained or derived from a donor, who belongs to the same species, but is different from the individual receiving the therapeutic cells. In some embodiments, the allogeneic therapeutic cell is an off-the-shelf therapeutic cell, which is pre-manufactured, characterized, and made available for immediate administration to patients. In some embodiments, the allogeneic therapeutic cell is a “universal” therapeutic cell, which is derived from cells obtained from one or more donors or cell lines, and is used in adoptive cell therapy for other individuals of the same species.

In some embodiments, the therapeutic cell is derived from a primary cell. In some embodiments, the therapeutic cell is a primary cell isolated from an individual. In some embodiments, the therapeutic cell is propagated (such as proliferated and/or differentiated) from a primary cell isolated from an individual. In some embodiments, the therapeutic cell is of the hematopoietic lineage. In some embodiments, the primary cell is obtained from the thymus. In some embodiments, the primary cell is obtained from the lymph or lymph nodes (such as tumor draining lymph nodes). In some embodiments, the primary cell is obtained from the spleen. In some embodiments, the primary cell is obtained from the bone marrow. In some embodiments, the primary cell is obtained from the blood, such as the peripheral blood. In some embodiments, the primary cell is a Peripheral Blood Mononuclear Cell (PBMC). In some embodiments, the primary cell is derived from the blood plasma In some embodiments, the primary cell is derived from a tumor. In some embodiments, the primary cell is obtained from the mucosal immune system. In some embodiments, the primary cell is obtained from a biopsy sample.

In some embodiments, the therapeutic cell is derived from a cell line. In some embodiments, the therapeutic cell is obtained from a commercial cell line. In some embodiments, the therapeutic cell is propagated (such as proliferated and/or differentiated) from a cell line established from a primary cell isolated from an individual. In some embodiments, the cell line is mortal. In some embodiments, the cell line is immortalized. In some embodiments, the cell line is a tumor cell line, such as a leukemia or lymphoma cell line. In some embodiments, the cell line is a cell line derived from the PBMC. In some embodiments, the cell line is a stem cell line. In some embodiments, the cell line is NK-92.

In some embodiments, the therapeutic cell is an immune cell or a progenitor thereof. Exemplary immune cells useful for the present invention include, but are not limited to, dendritic cells (including immature dendritic cells and mature dendritic cells), T lymphocytes (such as naïve T cells, effector T cells, memory T cells, cytotoxic T lymphocytes, T helper cells, Natural Killer T cells, Treg cells, tumor infiltrating lymphocytes (TIL), and lymphokine-activated killer (LAK) cells), B cells, Natural Killer (NK) cells, γδ T cells (including, e.g., γ9δ2 T cells, δ1 T cells, δ3 T cells), as T cells, monocytes, macrophages, neutrophils, granulocytes, and combinations thereof. Subpopulations of immune cells can be defined by the presence or absence of one or more cell surface markers known in the art (e.g., CD3, CD4, CD8, CD19, CD20, CD11c, CD123, CD56, CD34, CD14, CD33, etc.). In the cases that the pharmaceutical composition comprises a plurality of modified therapeutic cells, the therapeutic cells can be a specific subpopulation of an immune cell type, a combination of subpopulations of an immune cell type, or a combination of two or more immune cell types. In some embodiments, the immune cell is present in a homogenous cell population. In some embodiments, the immune cell is present in a heterogeneous cell population that is enhanced in the immune cell. In some embodiments, the therapeutic cell is a lymphocyte. In some embodiments, the therapeutic cell is not a lymphocyte. In some embodiments, the therapeutic cell is suitable for adoptive cell therapy. In some embodiments, the therapeutic cell is a PBMC. In some embodiments, the therapeutic cell is an immune cell derived from a PBMC. In some embodiments, the therapeutic cell is a T cell. In some embodiment, the therapeutic cell is a CD4⁺ T cell (also known as helper T cell). In some embodiments, the therapeutic cell is a CD8⁺ T cell (also known as cytotoxic T cell). In some embodiments, the therapeutic cell is a T cell expressing TCRα and TCRβ chains (i.e., as T cell). In some embodiments, the therapeutic cell is a T cell expressing TCRγ and TCRδ chains (i.e., γδ T cell). In some embodiments, the therapeutic cell is a γ9δ2 T cell. In some embodiments, the therapeutic cell is a 61 T cell. In some embodiments, the therapeutic cell is a δ3 T cell. In some embodiments, the therapeutic cell is a B cell. In some embodiments, the therapeutic cell is an NK cell. In some embodiments, the therapeutic cell is an NK-T cell. In some embodiments, the therapeutic cell is a dendritic cell (DC). In some embodiments, the therapeutic cell is a DC-activated T cell.

In some embodiments, there is provided a modified immune cell comprising a first heterologous nucleic acid sequence encoding a sICP47 protein or a functional variant thereof. In some embodiments, the first heterologous nucleic acid sequence encodes a naturally occurring sICP47 protein. In some embodiments, the first heterologous nucleic acid sequence encodes an engineered sICP47 protein comprising an active domain of a naturally occurring sICP47 protein. In some embodiments, the naturally occurring sICP47 protein is from a herpesvirus selected from the group consisting of Simian Agent 8 (SA8), Cercopithecine herpesvirus 16 (CeHV-16) and Cercopithecine herpesvirus 1 (CeHV-1). In some embodiments, the first heterologous nucleic acid sequence encodes a sICP47 of SA8 or a functional variant thereof. In some embodiments, the first heterologous nucleic acid sequence encodes a sICP47 of CeHV-16 or a functional variant thereof. In some embodiments, the sICP47 protein or functional variant thereof comprises an amino acid sequence having at least about 80% (e.g., at least about any one of 85%, 90%, 92%, 95%, 98%, 99%, or more) sequence identity to an amino acid sequence selected from the group consisting of SEQ ID NOs: 1-3. In some embodiments, the sICP47 protein or functional variant thereof comprises an amino acid sequence having at least about 80% (e.g., at least about any one of 85%, 90%, 92%, 95%, 98%, 99%, or more) sequence identity to an amino acid sequence selected from the group consisting of SEQ ID NOs: 4-6 and 24-25. In some embodiments, the immune cell is selected from the group consisting of cytotoxic T cell, helper T cell, NK cell, NK-T cell, αβ T cell, γδ T cell, tumor-infiltrating T cell, dendritic cell (DC)-activated T cell, and peripheral blood mononuclear cell (PBMC). In some embodiments, the modified immune cell is a T cell. In some embodiments, the modified immune cell is a T cell. In some embodiments, the modified immune cell is a γδ T cell comprising, such as a γ9δ2 T cell, a δ1 T cell, or a δ3 T cell.

In some embodiments, there is provided a modified immune cell comprising a first heterologous nucleic acid sequence encoding a sICP47 protein or a functional variant thereof and a second heterologous nucleic acid sequence encoding an agonist agent of an NK cell inhibitory receptor (e.g., NKG2A receptor). In some embodiments, the first heterologous nucleic acid sequence encodes a naturally occurring sICP47 protein. In some embodiments, the first heterologous nucleic acid sequence encodes an engineered sICP47 protein comprising an active domain of a naturally occurring sICP47 protein. In some embodiments, the naturally occurring sICP47 protein is from a herpesvirus selected from the group consisting of Simian Agent 8 (SA8), Cercopithecine herpesvirus 16 (CeHV-16) and Cercopithecine herpesvirus 1 (CeHV-1). In some embodiments, the first heterologous nucleic acid sequence encodes a sICP47 of SA8 or a functional variant thereof. In some embodiments, the first heterologous nucleic acid sequence encodes a sICP47 of CeHV-16 or a functional variant thereof. In some embodiments, the sICP47 protein or functional variant thereof comprises an amino acid sequence having at least about 80% (e.g., at least about any one of 85%, 90%, 92%, 95%, 98%, 99%, or more) sequence identity to an amino acid sequence selected from the group consisting of SEQ ID NOs: 1-3. In some embodiments, the sICP47 protein or functional variant thereof comprises an amino acid sequence having at least about 80% (e.g., at least about any one of 85%, 90%, 92%, 95%, 98%, 99%, or more) sequence identity to an amino acid sequence selected from the group consisting of SEQ ID NOs: 4-6 and 24-25. In some embodiments, the agonist agent is an antibody or antigen-binding fragment thereof that specifically binds to the NK cell inhibitory receptor. In some embodiments, the agonist agent is a membrane-bound antibody that specifically binds NKG2A. In some embodiments, the agonist agent is a ligand of the NK cell inhibitory receptor. In some embodiments, the agonist agent comprises a VH comprising a HC-CDR1 comprising SEQ ID NO: 7, a HC-CDR2 comprising SEQ ID NO: 8, a HC-CDR3 comprising SEQ ID NO: 9, and a VL comprising a LC-CDR1 comprising SEQ ID NO: 10, a LC-CDR2 comprising SEQ ID NO: 11, and a LC-CDR3 comprising SEQ ID NO: 12. In some embodiments, the agonist agent comprises a VH comprising SEQ ID NO: 13 and a VL comprising SEQ ID NO: 14. In some embodiments, the agonist agent is a polypeptide comprising from the N-terminus to the C-terminus a single-chain variable fragment (scFv) that specifically binds to NKG2A, a CD8α hinge sequence and a CD8α transmembrane sequence. In some embodiments, the immune cell is selected from the group consisting of cytotoxic T cell, helper T cell, NK cell, NK-T cell, αβ T cell, γδ T cell, tumor-infiltrating T cell, dendritic cell (DC)-activated T cell, and peripheral blood mononuclear cell (PBMC). In some embodiments, the modified immune cell is a T cell. In some embodiments, the modified immune cell is a γδ T cell comprising, such as a γ9δ2 T cell, a δ1 T cell, or a δ3 T cell.

In some embodiments, the modified therapeutic cell is a stem cell or derived from a stem cell. In some embodiments, the stem cell is a totipotent stem cell. In some embodiments, the stem cell is a pluripotent stem cell. In some embodiments, the stem cell is a unipotent stem cell. In some embodiments, the stem cell is a progenitor cell. In some embodiments, the stem cell is an embryonic stem cell (ESC). In some embodiments, the stem cell is hematopoietic stem cell (HSC). In some embodiments, the stem cell is a mesenchymal stem cell. In some embodiments, the stem cell is an induced pluripotent stem cell (iPSC).

In some embodiments, the modified therapeutic cell comprises one or more heterologous nucleic acid sequences. The modified immune cell may comprise any number (such as any of 1, 2, 3, 4, 5, 10, 50, 100, 1000, or more) of the heterologous nucleic acid sequence(s). In some embodiments, the modified immune cell comprises a single copy of the heterologous nucleic acid sequence. In some embodiments, the modified immune cell comprises a plurality of copies of the heterologous nucleic acid sequence(s).

In some embodiments, the modified therapeutic cell (e.g., immune cell or stem cell) further comprises a second heterologous nucleic acid sequence encoding an engineered receptor. In some embodiments, the modified therapeutic cell expresses two or more engineered receptors. In some embodiments, the modified therapeutic cell expresses one or more engineered receptors selected from the group consisting of a chimeric antigen receptor (CAR), a recombinant T cell receptor (TCR), a T-cell antigen coupler (TAC) receptor and a TCR fusion protein (TFP). In some embodiments, the therapeutic cell is a CAR-T cell. In some embodiments, the therapeutic cell is an allogeneic CAR-T cell. In some embodiments, the therapeutic cell is a UCAR-T cell. In some embodiments, the therapeutic cell is a TCR-T cell. In some embodiments, the modified immune cells express a CAR and a TFP. In some embodiments, the modified immune cells express a CAR and a recombinant TCR. In some embodiments, the modified immune cells express a CAR and a TAC receptor. In some embodiments, the modified immune cells express a recombinant TCR and a TAC receptor. In some embodiments, the modified immune cell further comprises at least one additional heterologous nucleic acid sequence, for example, a third heterologous nucleic acid sequence encoding an immunomodulatory agent, such as a co-stimulatory molecule, a cytokine, a chemokine, and/or an immune checkpoint inhibitor.

In some embodiments, there is provided a modified immune cell or progenitor thereof comprising a first heterologous nucleic acid sequence encoding a sICP47 protein or a functional variant thereof, and a second heterologous nucleic acid sequence encoding an engineered receptor. In some embodiments, the first heterologous nucleic acid sequence encodes a naturally occurring sICP47 protein. In some embodiments, the first heterologous nucleic acid sequence encodes an engineered sICP47 protein comprising an active domain of a naturally occurring sICP47 protein. In some embodiments, the naturally occurring sICP47 protein is from a herpesvirus selected from the group consisting of Simian Agent 8 (SA8), Cercopithecine herpesvirus 16(CeHV-16) and Cercopithecine herpesvirus 1 (CeHV-1). In some embodiments, the first heterologous nucleic acid sequence encodes a sICP47 of SA8 or a functional variant thereof. In some embodiments, the first heterologous nucleic acid sequence encodes a sICP47 of CeHV-16 or a functional variant thereof. In some embodiments, the sICP47 protein or functional variant thereof comprises an amino acid sequence having at least about 80% (e.g., at least about any one of 85%, 90%, 92%, 95%, 98%, 99%, or more) sequence identity to an amino acid sequence selected from the group consisting of SEQ ID NOs: 1-3. In some embodiments, the sICP47 protein or functional variant thereof comprises an amino acid sequence having at least about 80% (e.g., at least about any one of 85%, 90%, 92%, 95%, 98%, 99%, or more) sequence identity to an amino acid sequence selected from the group consisting of SEQ ID NOs: 4-6 and 24-25. In some embodiments, the engineered receptor is selected from the group consisting of a chimeric antigen receptor (CAR), a recombinant T cell receptor (TCR), a T-cell antigen coupler (TAC) receptor and a TCR fusion protein (TFP). In some embodiments, the engineered receptor targets a tumor antigen selected from the group consisting of CD19, BCMA, NY-ESO-1, VEGFR2, MAGE-A3, CD20, CD22, CD33, CD38, CEA, EGFR, GD2, HER2, IGF1R, mesothelin, PSMA, ROR1, WT1 and combinations thereof. In some embodiments, the immune cell is selected from the group consisting of cytotoxic T cell, helper T cell, NK cell, NK-T cell, as T cell, γδ T cell, tumor-infiltrating T cell, DC-activated T cell, and PBMC. In some embodiments, the modified immune cell is a T cell. In some embodiments, the modified immune cell is a γδ T cell. In some embodiments, the modified immune cell is a γ9δ2 T cell. In some embodiments, the modified immune cell is a δ1 T cell. In some embodiments, the modified immune cell is a δ3 T cell. In some embodiments, the modified progenitor cell is HSC.

In some embodiments, there is provided a modified immune cell comprising a first heterologous nucleic acid sequence encoding a sICP47 protein or a functional variant thereof, a second heterologous nucleic acid sequence encoding an agonist agent of an NK cell inhibitory receptor (e.g., NKG2A receptor), and a third heterologous nucleic acid sequence encoding an engineered receptor. In some embodiments, the first heterologous nucleic acid sequence encodes a naturally occurring sICP47 protein. In some embodiments, the first heterologous nucleic acid sequence encodes an engineered sICP47 protein comprising an active domain of a naturally occurring sICP47 protein. In some embodiments, the naturally occurring sICP47 protein is from a herpesvirus selected from the group consisting of Simian Agent 8 (SA8), Cercopithecine herpesvirus 16 (CeHV-16) and Cercopithecine herpesvirus 1 (CeHV-1). In some embodiments, the first heterologous nucleic acid sequence encodes a sICP47 of SA8 or a functional variant thereof. In some embodiments, the first heterologous nucleic acid sequence encodes a sICP47 of CeHV-16 or a functional variant thereof. In some embodiments, the sICP47 protein or functional variant thereof comprises an amino acid sequence having at least about 80% (e.g., at least about any one of 85%, 90%, 92%, 95%, 98%, 99%, or more) sequence identity to an amino acid sequence selected from the group consisting of SEQ ID NOs: 1-3. In some embodiments, the sICP47 protein or functional variant thereof comprises an amino acid sequence having at least about 80% (e.g., at least about any one of 85%, 90%, 92%, 95%, 98%, 99%, or more) sequence identity to an amino acid sequence selected from the group consisting of SEQ ID NOs: 4-6 and 24-25. In some embodiments, the agonist agent is an antibody or antigen-binding fragment thereof that specifically binds to the NK cell inhibitory receptor. In some embodiments, the agonist agent is a membrane-bound antibody that specifically binds NKG2A. In some embodiments, the agonist agent is a ligand of the NK cell inhibitory receptor. In some embodiments, the agonist agent comprises a VH comprising a HC-CDR1 comprising SEQ ID NO: 7, a HC-CDR2 comprising SEQ ID NO: 8, a HC-CDR3 comprising SEQ ID NO: 9, and a VL comprising a LC-CDR1 comprising SEQ ID NO: 10, a LC-CDR2 comprising SEQ ID NO: 11, and a LC-CDR3 comprising SEQ ID NO: 12. In some embodiments, the agonist agent comprises a VH comprising SEQ ID NO: 13 and a VL comprising SEQ ID NO: 14. In some embodiments, the agonist agent is a polypeptide comprising from the N-terminus to the C-terminus a single-chain variable fragment (scFv) that specifically binds to NKG2A, a CD8α hinge sequence and a CD8α transmembrane sequence. In some embodiments, the engineered receptor is selected from the group consisting of a chimeric antigen receptor (CAR), a recombinant T cell receptor (TCR), a T-cell antigen coupler (TAC) receptor and a TCR fusion protein (TFP). In some embodiments, the engineered receptor targets a tumor antigen selected from the group consisting of CD19, BCMA, NY-ESO-1, VEGFR2, MAGE-A3, CD20, CD22, CD33, CD38, CEA, EGFR, GD2, HER2, IGF1R, mesothelin, PSMA, ROR1, WT1 and combinations thereof. In some embodiments, the immune cell is selected from the group consisting of cytotoxic T cell, helper T cell, NK cell, NK-T cell, as T cell, γδ T cell, tumor-infiltrating T cell, DC-activated T cell, and PBMC. In some embodiments, the modified immune cell is a T cell. In some embodiments, the modified immune cell is a γδ T cell. In some embodiments, the modified immune cell is a γ9δ2 T cell. In some embodiments, the modified immune cell is a δ1 T cell. In some embodiments, the modified immune cell is a δ3 T cell. In some embodiments, the modified progenitor cell is HSC.

In some embodiments, there is provided a CAR-T cell comprising a first heterologous nucleic acid sequence encoding a first heterologous nucleic acid sequence encoding a sICP47 protein or a functional variant thereof, and a second heterologous nucleic acid sequence encoding a CAR. In some embodiments, the first heterologous nucleic acid sequence encodes a naturally occurring sICP47 protein. In some embodiments, the first heterologous nucleic acid sequence encodes an engineered sICP47 protein comprising an active domain of a naturally occurring sICP47 protein. In some embodiments, the naturally occurring sICP47 protein is from a herpesvirus selected from the group consisting of Simian Agent 8 (SA8), Cercopithecine herpesvirus 16 (CeHV-16) and Cercopithecine herpesvirus 1 (CeHV-1). In some embodiments, the first heterologous nucleic acid sequence encodes a sICP47 of SA8 or a functional variant thereof. In some embodiments, the first heterologous nucleic acid sequence encodes a sICP47 of CeHV-16 or a functional variant thereof. In some embodiments, the sICP47 protein or functional variant thereof comprises an amino acid sequence having at least about 80% (e.g., at least about any one of 85%, 90%, 92%, 95%, 98%, 99%, or more) sequence identity to an amino acid sequence selected from the group consisting of SEQ ID NOs: 1-3. In some embodiments, the sICP47 protein or functional variant thereof comprises an amino acid sequence having at least about 80% (e.g., at least about any one of 85%, 90%, 92%, 95%, 98%, 99%, or more) sequence identity to an amino acid sequence selected from the group consisting of SEQ ID NOs: 4-6 and 24-25. In some embodiments, the CAR targets a tumor antigen selected from the group consisting of CD19, BCMA, NY-ESO-1, VEGFR2, MAGE-A3, CD20, CD22, CD33, CD38, CEA, EGFR, GD2, HER2, IGF1R, mesothelin, PSMA, ROR1, WT1 and combinations thereof. In some embodiments, the CAR is an anti-BCMA CAR. In some embodiments, the CAR is an anti-CD19 CAR. In some embodiments, the CAR-T cell is a γδ T cell. In some embodiments, the CAR-T cell is a γ92 T cell. In some embodiments, the CAR-T cell is a δ1 T cell. In some embodiments, the CAR-T cell is a 63 T cell.

In some embodiments, there is provided a CAR-T cell comprising a first heterologous nucleic acid sequence encoding a first heterologous nucleic acid sequence encoding a sICP47 protein or a functional variant thereof, a second heterologous nucleic acid sequence encoding an agonist agent of an NK cell inhibitory receptor (e.g., NKG2A receptor), and a third heterologous nucleic acid sequence encoding a CAR. In some embodiments, the first heterologous nucleic acid sequence encodes a naturally occurring sICP47 protein. In some embodiments, the first heterologous nucleic acid sequence encodes an engineered sICP47 protein comprising an active domain of a naturally occurring sICP47 protein. In some embodiments, the naturally occurring sICP47 protein is from a herpesvirus selected from the group consisting of Simian Agent 8 (SA8), Cercopithecine herpesvirus 16 (CeHV-16) and Cercopithecine herpesvirus 1 (CeHV-1). In some embodiments, the first heterologous nucleic acid sequence encodes a sICP47 of SA8 or a functional variant thereof. In some embodiments, the first heterologous nucleic acid sequence encodes a sICP47 of CeHV-16 or a functional variant thereof. In some embodiments, the first heterologous nucleic acid sequence encodes a sICP47 of CeHV-16 or a functional variant thereof. In some embodiments, the sICP47 protein or functional variant thereof comprises an amino acid sequence having at least about 80% (e.g., at least about any one of 85%, 90%, 92%, 95%, 98%, 99%, or more) sequence identity to an amino acid sequence selected from the group consisting of SEQ ID NOs: 1-3. In some embodiments, the sICP47 protein or functional variant thereof comprises an amino acid sequence having at least about 80% (e.g., at least about any one of 85%, 90%, 92%, 95%, 98%, 99%, or more) sequence identity to an amino acid sequence selected from the group consisting of SEQ ID NOs: 4-6 and 24-25. In some embodiments, the agonist agent is an antibody or antigen-binding fragment thereof that specifically binds to the NK cell inhibitory receptor. In some embodiments, the agonist agent is a membrane-bound antibody that specifically binds NKG2A. In some embodiments, the agonist agent is a ligand of the NK cell inhibitory receptor. In some embodiments, the agonist agent comprises a VH comprising a HC-CDR1 comprising SEQ ID NO: 7, a HC-CDR2 comprising SEQ ID NO: 8, a HC-CDR3 comprising SEQ ID NO: 9, and a VL comprising a LC-CDR1 comprising SEQ ID NO: 10, a LC-CDR2 comprising SEQ ID NO: 11, and a LC-CDR3 comprising SEQ ID NO: 12. In some embodiments, the agonist agent comprises a VH comprising SEQ ID NO: 13 and a VL comprising SEQ ID NO: 14. In some embodiments, the agonist agent is a polypeptide comprising from the N-terminus to the C-terminus a single-chain variable fragment (scFv) that specifically binds to NKG2A, a CD8α hinge sequence and a CD8α transmembrane sequence. In some embodiments, the CAR targets a tumor antigen selected from the group consisting of CD19, BCMA, NY-ESO-1, VEGFR2, MAGE-A3, CD20, CD22, CD33, CD38, CEA, EGFR, GD2, HER2, IGF1R, mesothelin, PSMA, ROR1, WT1 and combinations thereof. In some embodiments, the CAR is an anti-BCMA CAR. In some embodiments, the CAR is an anti-CD19 CAR. In some embodiments, the CAR-T cell is a γδ T cell. In some embodiments, the CAR-T cell is a γ9δ2 T cell. In some embodiments, the CAR-T cell is a δ1 T cell. In some embodiments, the CAR-T cell is a δ3 T cell.

In some embodiments, the modified therapeutic cell is a T cell, such as allogeneic T cell. In some embodiments, the therapeutic cell is a TCRαβ⁺ T cell. In some embodiments, the therapeutic cell is a TCRγδ⁺ T cell. In some embodiments, the therapeutic cell is a γ9δ2 T cell. In some embodiments, the therapeutic cell is a 61 T cell. In some embodiments, the therapeutic cell is a δ3 T cell. In some embodiments, the therapeutic cell is a CAR-T cell. In some embodiments, the therapeutic cell expresses an anti-BCMA CAR. In some embodiments, the therapeutic cell is a CAR-T cell expressing a BSF17 CAR. In some embodiments, the therapeutic cell expresses an anti-CD19 CAR. In some embodiments, the therapeutic cell is a CAR-T cell expressing a CTL-019 CAR. In some embodiments, the therapeutic cell is a TCR-T cell. In some embodiments, the therapeutic cell is a T cell expressing a TAC receptor. In some embodiments, the therapeutic cell is a T cell expressing a TFP. In some embodiments, the therapeutic cell is a T cell expressing a combination of engineered receptors selected from the group consisting of CAR, TCR, TAC receptor and TFP.

In some embodiments, the therapeutic cell is a HSC, such as an allogeneic HSC. In some embodiments, the therapeutic cell is an HSC that expresses one or more therapeutic agents. In some embodiments, the therapeutic cell is an HSC that expresses one or more engineered receptors selected from the group consisting of CAR, TCR, TAC receptor and TFP. In some embodiments, the therapeutic cell is an HSC expressing one or more therapeutic agents other than engineered receptors. In some embodiments, the therapeutic cell is a viral vector-transduced HSC, such as retroviral or lentiviral transduced HSC.

In some embodiments, the therapeutic cell is an NK cell. In some embodiments, the therapeutic cell is an NK cell expressing one or more engineered receptors selected from the group consisting of CAR, TCR, TAC receptor and TFP.

In some embodiments, the therapeutic cell is an NK-T cell. In some embodiments, the therapeutic cell is an NK-T cell expressing one or more engineered receptors selected from the group consisting of CAR, TCR, TAC receptor and TFP.

In some embodiments, expression of the sICP47 protein or functional variant thereof downregulates cell surface expression of MHC molecules in the therapeutic cell.

In some embodiments, expression of the sICP47 protein or functional variant thereof reduces the expression level (e.g., RNA level or protein level) of the MHC molecule by no more than about 95%, such as no more than about any one of 10%, 15%, 20%, 25%, 30%, 35%, 40%, 45%, 50%, 55%, 60%, 65%, 70%, 75%, 80%, 85% or 90%. In some embodiments, expression of the sICP47 protein or functional variant thereof reduces the expression level (e.g., RNA level or protein level) of the MHC molecule by about any one of 10%, 15%, 20%, 25%, 30%, 35%, 40% h, 45%, 50%1a, 55%, 60%, 65%, 70%, 75%, 80%, 85%, 90%, including any values or ranges in between these values. In some embodiments, expression of the sICP47 protein or functional variant thereof reduces the expression level (e.g., RNA level or protein level) of the MHC molecule by about any one of 10%-20%, 20%-30%, 30%-40%, 40%-50%, 50%-60%, 60%-70%, 70%-80%, 80%-90%, 10%-25%, 25%-50%, 50%-75%, 75%-95%, 10%-30%, 30%-60%, 60%-90%, 10%-50%, 50%-95%, 25%-75%, 20%-40%, 40%-60%, 60%-80%, or 10%-90%. In some embodiments, the expression level is mRNA level, e.g., mRNA level of one or more subunits or chains of an MHC class I molecule or an MHC class 1H molecule. In some embodiments, the expression level is protein level, e.g., protein level of an MHC class I molecule, or an MHC class 1H molecule. The expression level may be steady-state expression level of the MHC molecule in the modified therapeutic cell. The steady-state expression level of the MHC molecule may be achieved in the modified therapeutic cell after the sICP47 protein or functional variant thereof is expressed in the therapeutic cell for at least about any one of 6 hours, 12 hours, 1 day, 2 days, 3 days, 1 week or more. The expression level of the MHC molecule can be measured using any known methods in the art. For example, mRNA expression level can be determined using quantitative polymerase chain reaction (qPCR), or fluorescence in situ hybridization (FISH) assays. Protein expression levels can be determined using Western blots, enzyme-linked immunosorbent assays (ELISA), or reporter assays.

In some embodiments, expression of the sICP47 protein or functional variant thereof reduces the expression level (e.g., RNA level or protein level) of MHC class I molecule(s) by no more than about 95%, such as no more than about any one of 10%, 15%, 20%, 25%, 30%, 35%, 40%, 45%, 50%, 55%, 60%, 65%, 70%, 75%, 80%, 85% or 90%. In some embodiments, expression of the sICP47 protein or functional variant thereof reduces the expression level of MHC class I molecule(s) by about any one of 10%, 15%, 20%, 25%, 30%, 35%, 40%, 45%, 50%, 55%, 60%, 65%, 70%, 75%, 80%, 85%, 90%, including any values or ranges in between these values. In some embodiments, expression of the sICP47 protein or functional variant thereof reduces the expression level (e.g., RNA level or protein level) of MHC class I molecule(s) by about any one of 10%-20%, 20%-30%, 30%-40%, 40%-50%, 50%-60%, 60%-70%, 70%-80%, 80%-90%, 10%-25%, 25%-50%, 50%-75%, 75%-95%, 10%-30%, 30%-60%, 60%-90%, 10%-50%, 50%-95%, 25%-75%, 20%-40%, 40%-60%, 60%-80%, or 10%-90%.

In some embodiments, the modified therapeutic cell expresses MHC class I molecules. In some embodiments, the modified therapeutic cell expresses MHC class H molecules. In some embodiments, the modified therapeutic cell expresses both MHC class I molecules and MHC class II molecules. In some embodiments, the MHC genes of the therapeutic cell are not genetically modified. For example, the modified therapeutic cell does not have a knock-out of its B2M gene.

In some embodiments, expression of the sICP47 protein or functional variant thereof reduces allo-reaction (e.g., cytotoxicity) mediated by allogeneic T cells or NK cells. In some embodiments, one or more HLA alleles of the allogeneic T cell or NK cell have mismatching allotypes compared to those of the modified therapeutic cell. In some embodiments, the mismatching alleles are for one or more class I HLA genes. In some embodiments, the mismatching alleles are for one or more class II HLA genes. In some embodiments, no more than any one of 7, 6, 5, 4, 3, 2, or 1 alleles of HLA-A, HLA-B, HLA-C and HLA-DRB1 of the allogeneic T cell or NK cell have mismatching allotypes compared to those of the modified therapeutic cell. In some embodiments, all tested HLA alleles (e.g., HLA-A, HLA-B, HLA-C and/or HLA-DRB1) of the allogeneic T cell or NK cell have mismatching allotypes compared to those of the modified therapeutic cell. In some embodiments, all tested HLA alleles (e.g., HLA-A, HLA-B, HLA-C and/or HLA-DRB1) of the allogeneic T cell or NK cell have matching allotypes compared to those of the modified therapeutic cell.

Recognition by T cell or NK cell can be determined using assays known in the art, for example, by in vitro co-culture assays in which the modified therapeutic cells are co-cultured with allogeneic T cells or NK cells (e.g., PBMCs) for a certain period of time, and recognition of the modified therapeutic cells by allogeneic T cells or NK cells can be assessed by measuring proliferation of T cells or NK cells (e.g., by CSFE staining), cytokine release (e.g., IFNγ, TNF, IL6 or 12) by T cells or NK cells, or survival of the modified therapeutic cells (e.g., using reporter assay such as LDH assay). In some embodiments, expression of the sICP47 protein or functional variant thereof reduces cytotoxicity by allogeneic T cells against the modified therapeutic cell by at least about any one of 10 fold, 20 fold, 50 fold, 100 fold, 200 fold, 500 fold, 1000 fold or more, including any values or ranges between these values, compared to an unmodified therapeutic cell that does not express the sICP47 protein or functional variant thereof. In some embodiments, expression of the sICP47 protein or functional variant thereof reduces cytotoxicity by allogeneic NK cells against the modified therapeutic cell by at least about any one of 10 fold, 20 fold, 50 fold, 100 fold, 200 fold, 500 fold, 1000 fold or more, including any values or ranges between these values, compared to an unmodified therapeutic cell that does not express the agent.

In vivo graft rejection or HvGD assays may also be used to assess allogeneic T cell or NK cell recognition of the modified therapeutic cells. For example, modified therapeutic cells and allogeneic T cells or NK cells may be transplanted into an immunodeficient mouse model, and persistence of the modified therapeutic cells in the mouse may be monitored over time using a reporter expressed by the modified therapeutic cells. In some embodiments, at least about 10%, such as at least about any one of 15%, 20%, 30%, 40%, 50%, 60%, 70%, 80%, 90% or more, including any values and ranges in between these values, of the modified therapeutic cells persist for a period of time in a host with allogeneic T cells or allogeneic NK cells. The period of time may be at least about any one of 1 week, 2 weeks, 3 weeks, 1 month, 2 months, 3 months, 6 months, 1 year or longer.

In some embodiments, there is provided a method of down-regulating expression of MHC class I (e.g., HLA-I) molecule on a modified therapeutic cell, comprising introducing into a precursor cell a first heterologous nucleic acid sequence encoding a sICP47 or a functional variant thereof. In some embodiments, the method further comprises introducing into the precursor cell a second heterologous nucleic acid sequence encoding an agonist agent of an NK cell inhibitory receptor, such as a membrane-bound anti-NKG2A antibody. In some embodiments, expression of the sICP47 or functional variant thereof down-regulates the expression level of MHC class I molecules on the modified therapeutic cell by at least about 10%, such as at least about any one of 15%, 20%, 30%, 40%, 50%, 60%, 70%, 80%, 90% or more, including any values and ranges in between these values, compared to the expression level of MHC class I molecules on the precursor cell.

As used herein, “down-regulating” the expression of a molecule (e.g., a gene) means suppressing transcription or translation of the molecule, which does not encompass knockout or complete depletion of the molecule. The expression level (e.g., RNA or protein level) of a molecule may be down-regulated to less than about any one of 90%, 80%, 70%, 60%, 50%, 40%, 30%, 20%, 10% or less compared to the wildtype level (e.g., RNA or protein level) of the molecule.

Also provided are methods of producing the modified therapeutic cells described herein. Nucleic acid(s) comprising the heterologous nucleic acid sequence(s) described herein may be transiently or stably incorporated in the modified therapeutic cells. In some embodiments, the nucleic acid(s) is transiently expressed in the modified therapeutic cell. For example, the nucleic acid(s) may be present in the nucleus of the modified therapeutic cell in an extrachromosomal array. The nucleic acid(s) may be introduced into the modified therapeutic cell using any transfection or transduction methods known in the art, including viral or non-viral methods. Exemplary non-viral transfection methods include, but are not limited to, chemical-based transfection, such as using calcium phosphate, dendrimers, liposomes, or cationic polymers (e.g., DEAE-dextran or polyethylenimine); non-chemical methods, such as electroporation, cell squeezing, sonoporation, optical transfection, impalefection, protoplast fusion, hydrodynamic delivery, or transposons; particle-based methods, such as using a gene gun, magnectofection or magnet assisted transfection, particle bombardment; and hybrid methods, such as nucleofection.

In some embodiments, the heterologous nucleic acid sequence(s) is present in the genome of the modified therapeutic cell. For example, nucleic acid(s) comprising the heterologous nucleic acid sequence(s) may be integrated into the genome of the modified therapeutic cell by any methods known in the art, including, but not limited to, virus-mediated integration, random integration, homologous recombination methods, and site-directed integration methods, such as using site-specific recombinase or integrase, transposase, Transcription activator-like effector nuclease (TALEN®), CRISPR/Cas9, and zinc-finger nucleases. In some embodiments, the heterologous nucleic acid sequence(s) is integrated in a specifically designed locus of the genome of the modified therapeutic cell. In some embodiments, the heterologous nucleic acid sequence(s) is integrated in an integration hotspot of the genome of the modified therapeutic cell. In some embodiments, the heterologous nucleic acid sequence(s) is integrated in a random locus of the genome of the modified therapeutic cell. In the cases that multiple copies of the heterologous nucleic acid sequence(s) are present in a single modified therapeutic cell, the heterologous nucleic acid sequences may be integrated in a plurality of loci of the genome of the modified therapeutic cell.

Precursor immune cells can be prepared using a variety of methods known in the art. For example, primary immune cells, such as T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, immune cells (such as T cells) can be obtained from a unit of blood collected from an individual using any number of techniques known in the art, such as FICOLL™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS), or a wash solution lacking divalent cations, such as calcium and magnesium. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In some embodiments, primary T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA, and CD45RO cells, can be further isolated by positive or negative selection techniques. For example, in one embodiment, T cells are isolated by incubation with anti-CD3/anti-CD28 (i.e., 3×28)-conjugated beads, such as DYNABEADS® M-450 CD3/CD28 T, for a time period sufficient for positive selection of the desired T cells.

In some embodiments, a T cell population may further be enriched by negative selection using a combination of antibodies directed to surface markers unique to the negatively selected cells. For example, one method involves cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells, which typically express CD4⁺, CD25⁺, CD62L^{hi} GITR⁺, and FoxP3⁺.

Methods of introducing vectors or nucleic acids into a therapeutic cell (such as a precursor immune cell) are known in the art. The vectors or nucleic acids can be transferred into a therapeutic cell by physical, chemical, or biological methods.

Physical methods for introducing the vector(s) or nucleic acid(s) into a therapeutic cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001) Molecular Cloning: A Laboratory Manual, Cold Spring Harbor Laboratory, New York. In some embodiments, the vector is introduced into the cell by electroporation.

Biological methods for introducing the vector(s) or nucleic acid(s) into a therapeutic cell include the use of DNA and RNA vectors. Viral vectors have become the most widely used method for inserting genes into mammalian, e.g., human cells.

Chemical means for introducing the vector(s) or nucleic acid(s) into a therapeutic cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

In some embodiments, the transduced or transfected precursor immune cell is propagated ex vivo after introduction of the heterologous nucleic acid(s). In some embodiments, the transduced or transfected precursor immune cell is cultured to propagate for at least about any of 1 day, 2 days, 3 days, 4 days, 5 days, 6 days, 7 days, 10 days, 12 days, or 14 days. In some embodiments, the transduced or transfected precursor immune cell is cultured for no more than about any of 1 day, 2 days, 3 days, 4 days, 5 days, 6 days, 7 days, 10 days, 12 days, or 14 days. In some embodiments, the transduced or transfected precursor immune cell is further evaluated or screened to select the modified immune cell.

Reporter genes may be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al. FEBS Letters 479: 79-82 (2000)).

Other methods to confirm the presence of the heterologous nucleic acid(s) in the precursor immune cell, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots).

### A. Simian ICP47

The modified therapeutic cells described herein express a simian ICP47 (sICP47) protein or a functional variant thereof.

In some embodiments, the sICP47 protein is derived from ICP47 of SA8. The SA8 sICP47 protein is a 78-amino acid protein encoded by strain Simian Agent 8 of the herpes simplex virus type 2, with an active domain comprising amino acid residues 3-36. In some embodiments, the SA8 sICP47 has the amino acid sequence of SEQ ID NO: 4.

In some embodiments, the sICP47 protein comprises an active domain of ICP47 of SA8 or a functional variant thereof. In some embodiments, the sICP47 protein or fictional variant thereof comprises an amino acid sequence having at least 80% (e.g., at least about any one of 85%, 90°/a, 92%, 95%, 98%, 99%, or more) sequence identity to the amino acid sequence of SEQ ID NO: 1. In some embodiments, the sICP47 protein comprises the amino acid sequence of SEQ ID NO: 1. In some embodiments, the sICP47 protein consists of the amino acid sequence of SEQ ID NO: 1.

In some embodiments, the sICP47 protein comprises full-length sICP47 of SA8 or a functional variant thereof. In some embodiments, the sICP47 protein or functional variant thereof comprises an amino acid sequence having at least 80% (e.g., at least about any one of 85%, 90%, 92%, 95%, 98%, 99%, or more) sequence identity to the amino acid sequence of SEQ ID NO: 4. In some embodiments, the sICP47 protein comprises the amino acid sequence of SEQ ID NO: 4. In some embodiments, the sICP47 protein consists of the amino acid sequence of SEQ ID NO: 4.

In some embodiments, the sICP47 protein is derived from ICP47 of Cercopithecine herpesvirus 16 (CeHV-16). In some embodiments, the sICP47 of CeHV-16 has the amino acid sequence of SEQ ID NO: 5, including an active domain comprising the amino acid sequence of SEQ ID NO: 2.

In some embodiments, the sICP47 protein comprises an active domain of ICP47 of CeHV-16 or a functional variant thereof. In some embodiments, the sICP47 protein or functional variant thereof comprises an amino acid sequence having at least 80% (e.g., at least about any one of 85%, 90%, 92%, 95%, 98%, 99%, or more) sequence identity to the amino acid sequence of SEQ ID NO: 2. In some embodiments, the sICP47 protein comprises the amino acid sequence of SEQ ID NO: 2. In some embodiments, the sICP47 protein consists of the amino acid sequence of SEQ ID NO: 2.

In some embodiments, the sICP47 protein comprises full-length sICP47 of CeHV-16 or a functional variant thereof. In some embodiments, the sICP47 protein or functional variant thereof comprises an amino acid sequence having at least 80% (e.g., at least about any one of 85%, 90%, 92%, 95%, 98%, 99%, or more) sequence identity to the amino acid sequence of SEQ ID NO: 5. In some embodiments, the sICP47 protein comprises the amino acid sequence of SEQ ID NO: 5. In some embodiments, the sICP47 protein consists of the amino acid sequence of SEQ ID NO: 5.

In some embodiments, the sICP47 protein is derived from ICP47 of Cercopithecine herpesvirus 1 (CeHV-1). In some embodiments, the sICP47 of CeHV-1 has the amino acid sequence of SEQ ID NO: 6, including an active domain comprising the amino acid sequence of SEQ ID NO: 3.

In some embodiments, the sICP47 protein comprises an active domain of ICP47 of CeHV-1 or a functional variant thereof. In some embodiments, the sICP47 protein or functional variant thereof comprises an amino acid sequence having at least 80% (e.g., at least about any one of 85%, 90%, 92%, 95%, 98%, 99%, or more) sequence identity to the amino acid sequence of SEQ ID NO: 3. In some embodiments, the sICP47 protein comprises the amino acid sequence of SEQ ID NO: 3. In some embodiments, the sICP47 protein consists of the amino acid sequence of SEQ ID NO: 3.

In some embodiments, the sICP47 protein comprises full-length sICP47 of CeHV-1 or a functional variant thereof. In some embodiments, the sICP47 protein or functional variant thereof comprises an amino acid sequence having at least 80% (e.g., at least about any one of 85%, 90%, 92%, 95%, 98%, 99%, or more) sequence identity to the amino acid sequence of SEQ ID NO: 6. In some embodiments, the sICP47 protein comprises the amino acid sequence of SEQ ID NO: 6. In some embodiments, the sICP47 protein consists of the amino acid sequence of SEQ ID NO: 6.

In some embodiments, the sICP47 protein is derived from ICP47 of Macacine alphaherpesvirus 1. In some embodiments, the sICP47 of Macacine alphaherpesvirus 1 has the amino acid sequence of SEQ ID NO: 24.

In some embodiments, the sICP47 protein comprises an active domain of ICP47 of Macacine alphaherpesvirus 1 or a functional variant thereof. In some embodiments, the sICP47 protein comprises full-length sICP47 of Macacine alphaherpesvirus 1 or a functional variant thereof. In some embodiments, the sICP47 protein or functional variant thereof comprises an amino acid sequence having at least 80% (e.g., at least about any one of 85%, 90%, 92%, 95%, 98%, 99%, or more) sequence identity to the amino acid sequence of SEQ ID NO: 24. In some embodiments, the sICP47 protein comprises the amino acid sequence of SEQ ID NO: 24. In some embodiments, the sICP47 protein consists of the amino acid sequence of SEQ ID NO: 24.

In some embodiments, the sICP47 protein is derived from ICP47 of Pappine alphaherpesvirus 2. In some embodiments, the sICP47 of Pappine alphaherpesvirus 2 has the amino acid sequence of SEQ ID NO: 25.

In some embodiments, the sICP47 protein comprises an active domain of ICP47 of Pappine alphaherpesvirus 2 or a functional variant thereof. In some embodiments, the sICP47 protein comprises full-length sICP47 of Pappine alphaherpevirus 2 or a functional variant thereof. In some embodiments, the sICP47 protein or functional variant thereof comprises an amino acid sequence having at least 80% (e.g., at least about any one of 85%, 90%, 92%, 95%, 98%, 99%, or more) sequence identity to the amino acid sequence of SEQ ID NO: 25. In some embodiments, the sICP47 protein comprises the amino acid sequence of SEQ ID NO: 25. In some embodiments, the sICP47 protein consists of the amino acid sequence of SEQ ID NO: 25.

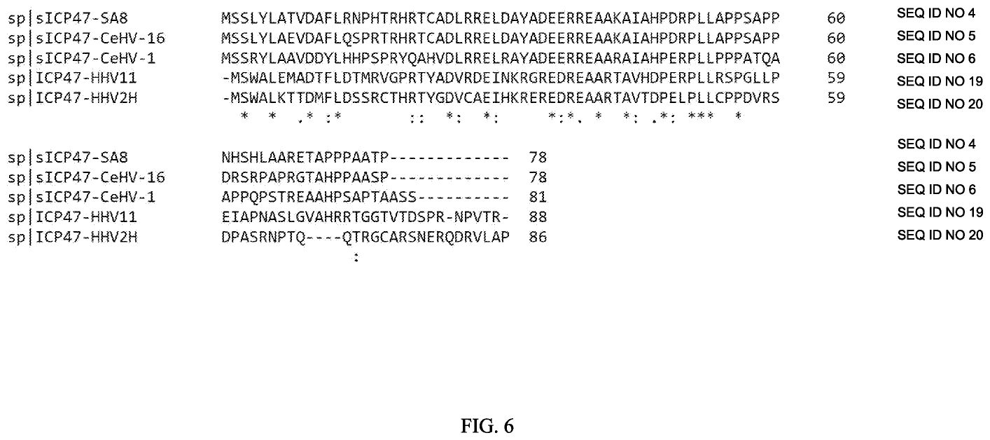

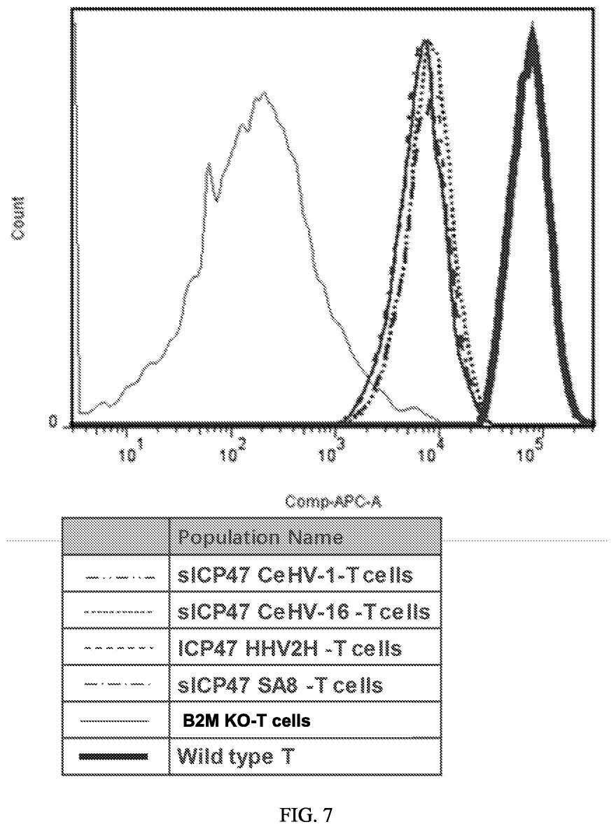

The simian ICP47 proteins described herein have low sequence identities to ICP47 proteins from human herpesviruses. FIG. 6 shows sequence alignments of three sICP47 proteins and two human ICP47 proteins. Table 2 below shows pairwise sequence identities among the various ICP47 proteins. FIG. 7 shows the simian ICP47 proteins from a herpesvirus selected from the group consisting of Simian Agent 8 (SA8), Cercopithecine herpesvirus 16 (CeHV-16) and Cercopithecine herpesvirus 1 (CeHV-1) can down-regulate the expression of HLA-ABC.

TABLE 2
Sequence Identity between strains of sICP47s and human ICP47s

| Sequence | sICP47 | SICP47 | SICP47 | ICP47 | ICP47 |
|---|---|---|---|---|---|
| Identity (%) | SA8 | CeHV-16 | CeHV-1 | HHV11 | HHV2H |
| sICP47 SA8 | 100.00 | 84.62 | 57.69 | 31.17 | 28.77 |
| (SEQ ID NO: 4) | | | | | |
| sICP47 CeHV-16 | 84.62 | 100.00 | 57.69 | 35.06 | 32.88 |
| (SEQ ID NO: 5) | | | | | |
| sICP47 CeHV-1 | 57.69 | 57.69 | 100.00 | 25.00 | 26.32 |
| (SEQ ID NO: 6) | | | | | |
| ICP47 HHV11 | 31.17 | 35.06 | 25.00 | 100.00 | 50.00 |
| (SEQ ID NO: 19) | | | | | |
| ICP47 HHV2H | 28.77 | 32.88 | 26.32 | 50.00 | 100.00 |
| (SEQ ID NO: 20) | | | | | |

Without being bound by theory, the active domain of sICP47 blocks peptide binding to TAP, as well as suppress peptide transport by TAP. sICP47 is a viral protein that plays a role in the inhibition of the host immune response, by blocking antigen processing and presentation in the host cells (see J Exp Med. 1997 May 5; 185(9): 1565-1572). In general, ICP47 protein binds specifically to TAP (transporters associated with antigen processing) in the infected cells. The interactions between sICP47 and TAP blocks peptide binding and translocation by TAP, and subsequently inhibits loading of peptides onto MHC class I molecules. Without peptides bound, MHC I molecules are subjected to proteasomal degradation in the endoplasmic reticulum. Thus, infected cells are masked for immune recognition by cytotoxic T-lymphocytes. See, for example, Journal of virology 74.10 (2000): 4465-4473.

Central to adaptive immunity is the interaction between the as T cell antigen receptor (TCR) on T cells and peptides presented by the major histocompatibility complex (MHC) molecules on the surface of target cells. The human MHC is called the HLA (Human Leukocyte Antigen). The MHC molecules are highly polymorphic. Allogeneic cells express MHC class I and MHC class II molecules, which may cause graft rejection when administered to an MHC-mismatched patient. The patient's own T cells and NK cells could reject the infused therapeutic cells, resulting in diminished therapeutic efficacy. See, for example, Dao M et al., Cancer Res (2018) 78: 3588; Patel S et al. Front Oncol. (2019); 9: 196.

Two major classes of MHC molecules, MHC class I and MHC class II molecules, determine the recognition of foreign cells by T-lymphocytes in a host. MHC class I molecules are expressed on the surface of almost all nucleated cells, including immune cells and HSCs. The MHC class I molecule is a heterodimer comprising a highly polymorphic alpha heavy chain, which is non-covalently associated with a conserved light chain called beta-2 microglobulin (B2M). The heavy chains of HLA class I molecules are encoded by major class I HLA genes HLA-A, HLA-B and HLA-C, and minor class I HLA genes HLA-E, HLA-F and HLA-G. The conserved β2-microglobulin binds with a subunit encoded by a major or minor class I HLA gene to produce a functional HLA class I heterodimer on the surface of a cell. MHC class I molecules present endogenously synthesized peptides to host CD8+ T lymphocytes.

MHC class II molecules are expressed on B lymphocytes, antigen-presenting cells (e.g., monocytes, macrophages, and dendritic cells), and activated T cells. Class II HLA genes include HLA-DP (i.e., HLA-DPA1 encoding alpha chain, and HLA-DPB1 encoding beta chain), HLA-DQ (i.e., HLA-DQA1 encoding alpha chain, and HLA-DQB1 encoding beta chain), and HLA-DR (i.e., HLA-DRA encoding alpha chain, and HLA-DRB1, DRB3, DRB4 and DRB5 encoding beta chain). MHC II molecules function by presenting antigenic peptides to CD4+ T cells, which lead to mutual cell activation and propagation of the humoral immune response. See, Patel S. et al. Front Oncol. (2019); 9: 196.

MHC-I plays a critical role in the events leading to the rejection or acceptance of allografts. “Mechanism of cellular rejection in transplantation.” Pediatr Nephrol. 2010; 25(1): 61-74. A non-DNA binding protein, class II transactivator (CUTA), has been identified as the pivotal master regulator that regulates expression of MHC class II and antigen-processing genes in a tissue-specific and inducible manner.

HLA genes are highly polymorphic, and each HLA gene has many different alleles. HLA typing, HLA antibody screening and crossmatching can be carried out to assess whether a modified therapeutic cell has mismatched HLA allele(s) compared to those of host cells (such as allogeneic T cell or NK cell, or cells from an individual being treated), as recognition of foreign HLA by host T cells could trigger an immune response against adoptively transferred cells. Methods for HLA typing, antibody screening and crossmatching are known in the art. See, for example, Choo, Yonsei Medical Journal, 48(1): 11-23 (2007); and Althaf et al., World Journal of Transplantation, 2017, 7(6): 339-348, which are incorporated herein by reference in their entirety.

HLA typing can be done using serologic or molecular typing methods. In a serologic HLA typing assay, a tray containing sera with antibodies to a multitude of known HLA alleles is used. For typing, recipient lymphocytes are introduced into the tray wells contacting sera, complement and dye. In tray wells where antibodies can bind to the antigens on the surface of lymphocytes, complement is activated. This results in complement pathways triggered resulting in cell death, ultimately allowing the dye to enter the cell. Tray wells with significant cell death are then identified under phase contrast microscopy. Through a process of comparison and elimination of positive wells, the HLA type is assigned. Peripheral blood lymphocytes (PBLs) can be used for serologic typing of HLA-A, HLA-B and HLA-C. B cells isolated from PBLs can be used for serologic typing of HLA class II, such as HLA-DRB1. Molecular HLA typing can be carried out using PCR-based assays such as sequence-specific oligonucleotide probes (SSOP) or sequence-specific primer (SSP) method, or by DNA sequencing.

Without being bound by any theory or hypothesis, graft rejection is mediated by host T and NK cells. There are several proposed mechanisms for recognition of alloantigens on graft cells. Allo-MHC molecules can create many new pMHC complexes that can serve as ligands for various T cell clones. See, for example, Front Immunol. 13(3):184. The prevalence of either model in T cell allorecognition presumably depends upon the degree of heterogeneity (structural and/or conformational) between recipient and donor MHC molecules. Induction of the adaptive immune response to an allograft begins with recognition of alloantigen by recipient T cells, which is now well characterized and known to occur through three main processes known as the direct, the indirect, and the semi-direct pathways of antigen presentation, discussed below.

Direct Allorecognition: Naïve T cells located in lymph nodes can become activated through recognition of allogeneic MHC molecules displayed on donor passenger leukocytes. The direct T cell alloresponse is polyclonal in that it involves a large portion of the T cell repertoire (1-10%).

Indirect Allorecognition: T cells interact with donor peptides (derived from MHC and minor histocompatibility antigens) processed and presented by recipient APCs, thus indirectly activate allorecognition. This can be inhibited, for example, by expressing sICP47 in the cells.

Semi-Direct Allorecognition: T cells can also recognize intact donor MHC molecules on recipient APCs (MHC molecules transferred from donor to recipient APCs).

More specifically, MHC class I H chain and B2M assemble with peptide in a multimeric complex with calreticulin, ERp57, tapasin, and the TAP heterodimer in the ER. Certain viral proteins retard MHC class I egress or induce its turnover, in some cases by ejection of the molecules from the ER into the cytoplasm. Peptides are provided by proteasomal cleavage of ubiquitinated cytosolic proteins and TAP transport into the ER, and both TAP and the proteasome are known targets for viral interference. Within the ER, the peptides are N-terminally trimmed by ER aminopeptidase associated with Ag presentation (ERAAP). Once peptide is bound, the complete MHC class I molecule is released from ER chaperones and proceeds through the Golgi. Via vesicular transport, the MHC class I molecule reaches the cell surface where it can present peptide to CTL (see J Immunol, 2003, 171:4473-4478; and PNAS, 2003, 102: 5144-5149).

After the arrival of MHC class I molecules at the cell surface, the presence of certain virus proteins can cause the endocytosis of MHC class I molecules. For example, HIV-1 Nef binds MHC class I on its cytoplasmic tail and escorts it from the cell membrane into the endosomal compartment. These MHC molecules are subsequently degraded, or they are transported into the trans-Golgi with the assistance of protein transport proteins like phosphofurin acidic cluster sorting protein-1, adaptor protein complexes, and phosphoinositide 3-kinase (see J biomed biotechnol, Volume 2011, Article ID 724607).

Other viruses, such as human CMV (HCMV) invests heavily in products able to interfere with MHC class I. The HCMV unique short (US) genes (US2, US3, US6, and US11) all assist HCMV in evading MHC class I presentation. HCMV encodes a unique long (UL) region protein, UL18, which is an MHC class I homolog, capable of binding b2m and peptide.

Notably, Herpes Simplex Virus 1 (HSV-1) and HSV-2 encode a soluble cytoplasmic protein, ICP47, that associates with the peptide binding site formed by the C-terminal cytosolic domains of TAP1 and TAP2, thereby acting as a high-affinity competitor for peptide binding to the MHC class I molecules. See International Immunology, 9(19): 1115-1122.

Natural killer (NK) cells play an important role in the host response against viral infections and cancer development. They are able to kill virus-infected and tumor cells, and they produce different important cytokines that stimulate the antiviral and antitumor adaptive immune response. While, more reports described the virus NK cell evasion strategies, particularly obvious for cytomegaloviruses, and increasing evidence indicates that most, if not all, members of the herpesvirus family suppress NK cell activity to some extent (Steffi D. P. et al. (2018) Journal of Virology, 92(11): 2105-2117; and Cristina Cerboni et al. (2007) Journal of General Virology, 88, 242-250).

CD4 and CD8 are coreceptors known to bind class II and class I MHC molecules, respectively and to be involved in the activation and biology of T cells. Much of the effect of CD4 is apparently attributable to the recruitment of lck to the TCR-CD3 complex, which results in efficient T cell activation. In addition, there also is a significant positive effect on T cells, even with CD4 molecules that are unable to bind lck, and there appears to be an effect on TCR-ligand interaction as well (Janeway, C. A et al. Annu. Rev. Immunol. 10:645, 1992; Miceli, M. C. et al. Adv. Immunol. 53:59, 1993; Zamoyska, R. 1998. Curr. Opin. Immunol. 10:82). CD8 also greatly augments the response of class I MHC-specific T cells and is thought to stabilize TCR-peptide-MHC complexes by about 10-fold (31, 32, 33). Overall, each of these coreceptor molecules apparently has two roles: to stabilize TCR-ligand interactions physically and to aid in signaling by recruiting other molecules.

Evidence from transgenic mouse models suggests that CD8 plays a critical role in TCR binding and activation by peptide-MHC complex multimers (tetramers). With a human CTL clone specific for a tumor-associated MHC-peptide complex that the binding of tetramers to the TCR on these cells can be completely blocked by anti-human CD8 antibodies. This blockage was mediated by anti-CD8 Abs but not anti-CD3 Abs and was dose dependent. The blocking effect of the anti-CD8 Abs was attributable to directly inhibiting tetramer binding and was not attributable to Ab-mediated TCR-CD8 internalization and down-regulation.

### B. Engineered Receptor

The modified therapeutic cells described herein may express one or more engineered receptors. Exemplary engineered receptors include, but are not limited to, CAR, recombinant TCR, TAC receptor, and TFPs. In some embodiments, the engineered receptor comprises an extracellular domain that specifically binds to an antigen (e.g., a tumor antigen), a transmembrane domain, and an intracellular signaling domain. In some embodiments, the intracellular signaling domain comprises a primary intracellular signaling domain and/or a co-stimulatory domain. In some embodiments, the intracellular signaling domain comprises an intracellular signaling domain of a TCR co-receptor. In some embodiments, the engineered receptor is encoded by a heterologous nucleic acid operably linked to a promoter (such as a constitutive promoter or an inducible promoter). In some embodiments, the engineered receptor is introduced to the modified immune cell by inserting proteins into the cell membrane while passing cells through a microfluidic system, such as CELL SQUEEZE (see, for example, U.S. Patent Application Publication No. 20140287509). The engineered receptor may enhance the function of the modified therapeutic cells, such as by targeting the modified therapeutic cells (e.g., modified immune cells), by transducing signals, and/or by enhancing cytotoxicity of the modified therapeutic cells (e.g., modified immune cells). In some embodiments, the modified therapeutic cell does not express an engineered receptor, such as CAR, TCR, TAC receptor, or TFP.

In some embodiments, the engineered receptor comprises one or more specific binding domains that target at least one tumor antigen, and one or more intracellular effector domains, such as one or more primary intracellular signaling domains and/or co-stimulatory domains.

In some embodiments, the engineered receptor is a chimeric antigen receptor (CAR). Many chimeric antigen receptors are known in the art and may be suitable for the modified therapeutic cells of the present invention. CARs can also be constructed with a specificity for any cell surface marker by utilizing antigen binding fragments or antibody variable domains of, for example, antibody molecules. Any method for producing a CAR may be used herein. See, for example, U.S. Pat. Nos. 6,410,319, 7,446,191, 7,514,537, 9,765,342B2, WO 2002/077029, WO2015/142675, US2010/065818, US 2010/025177, US 2007/059298, WO2017025038A1, and Berger C. et al., J. Clinical Investigation 118: 1 294-308 (2008), which are hereby incorporated by reference.

In some embodiments, a CAR comprises an extracellular domain comprising at least one targeting domain that specifically binds at least one tumor antigen, a transmembrane domain, and an intracellular signaling domain. In some embodiments, the intracellular signaling domain generates a signal that promotes an immune effector function of the CAR-containing cell, e.g., a CAR-T cell. “Immune effector function or immune effector response” refers to function or response, e.g., of an immune effector cell, that enhances or promotes an immune attack of a target cell. For example, an immune effector function or response may refer to a property of a T or NK cell that promotes killing or the inhibition of growth or proliferation, of a target cell. Examples of immune effector function, e.g., in a CAR-T cell, include cytolytic activity (such as antibody-dependent cellular toxicity, or ADCC) and helper activity (such as the secretion of cytokines). In some embodiments, the CAR has an intracellular signaling domain with an attenuated immune effector function. In some embodiments, the CAR has an intracellular signaling domain having no more than about any of 90%, 80%, 70%, 60%, 50%, 40%, 30%, 20%, 10% or less of an immune effector function (such as cytolytic function against target cells) compared to a CAR having a full-length and wildtype CD3ζ and optionally one or more co-stimulatory domains. In some embodiments, the intracellular signaling domain generates a signal that promotes proliferation and/or survival of the CAR containing cell. In some embodiments, the CAR comprises one or more intracellular signaling domains selected from the signaling domains of CD28, CD137, CD3, CD27, CD40, ICOS, GITR, and OX40. The signaling domain of a naturally occurring molecule can comprise the entire intracellular (i.e., cytoplasmic) portion, or the entire native intracellular signaling domain, of the molecule, or a fragment or derivative thereof.

In some embodiments, the intracellular signaling domain of a CAR comprises a primary intracellular signaling domain. “Primary intracellular signaling domain” refers to cytoplasmic signaling sequence that acts in a stimulatory manner to induce immune effector functions. In some embodiments, the primary intracellular signaling domain contains a signaling motif known as Immunoreceptor Tyrosine-based Activation Motif or ITAM. In some embodiments, the primary intracellular signaling domain comprises a functional signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma RIIa, DAP10, and DAP 12. In some embodiments, the primary intracellular signaling domain comprises a nonfunctional or attenuated signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma RIIa, DAP10, and DAP 12. The nonfunctional or attenuated signaling domain can be a mutant signaling domain having a point mutation, insertion or deletion that attenuates or abolishes one or more immune effector functions, such as cytolytic activity or helper activity, including antibody-dependent cellular toxicity (ADCC). In some embodiments, the CAR comprises a nonfunctional or attenuated CD3 zeta (i.e. CD3ζ or CD3z) signaling domain. In some embodiments, the intracellular signaling domain does not comprise a primary intracellular signaling domain. An attenuated primary intracellular signaling domain may induce no more than about any of 90%, 80%, 70%, 60%, 50%, 40%, 30%, 20%, 10% or less of an immune effector function (such as cytolytic function against target cells) compared to CARs having the same construct, but with the wildtype primary intracellular signaling domain.

In some embodiments, the intracellular signaling domain of a CAR comprises one or more (such as any of 1, 2, 3, or more) co-stimulatory domains. “Co-stimulatory domain” can be the intracellular portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival. Co-stimulatory molecules are cell surface molecules other than antigen receptors or their ligands that contribute to an efficient immune response. A co-stimulatory molecule can be represented in the following protein families: TNF receptor proteins, Immunoglobulin-like proteins, cytokine receptors, integrins, signaling lymphocytic activation molecules (SLAM proteins), and activating NK cell receptors. Co-stimulatory molecules include, but are not limited to an MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such co-stimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CDIOO (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

In some embodiments, the CAR comprises a single co-stimulatory domain. In some embodiments, the CAR comprises two or more co-stimulatory domains. In some embodiments, the intracellular signaling domain comprises a functional primary intracellular signaling domain and one or more co-stimulatory domains. In some embodiments, the CAR does not comprise a functional primary intracellular signaling domain (such as CD3ζ). In some embodiments, the CAR comprises an intracellular signaling domain consisting of or consisting essentially of one or more co-stimulatory domains. In some embodiments, the CAR comprises an intracellular signaling domain consisting of or consisting essentially of a nonfunctional or attenuated primary intracellular signaling domain (such as a mutant CD3ζ) and one or more co-stimulatory domains. Upon binding of the targeting domain to tumor antigen, the co-stimulatory domains of the CAR may transduce signals for enhanced proliferation, survival and differentiation of the engineered immune cells having the CAR (such as T cells), and inhibit activation induced cell death. In some embodiments, the one or more co-stimulatory signaling domains are derived from one or more molecules selected from the group consisting of CD27, CD28, 4-1BB (i.e., CD137), OX40, CD30, CD40, CD3, lymphocyte function-associated antigen-1(LFA-1), CD2, CD7, LIGHT, NKG2C, B7-H3 and ligands that specially bind to CD83.

In some embodiments, the intracellular signaling domain of the CAR comprises a co-stimulatory signaling domain derived from CD28. In some embodiments, the intracellular signaling domain comprises a cytoplasmic signaling domain of CD3 and a co-stimulatory signaling domain of CD28. In some embodiments, the intracellular signaling domain in the chimeric receptor of the present application comprises a co-stimulatory signaling domain derived from 4-1BB (i.e., CD137). In some embodiments, the intracellular signaling domain comprises a cytoplasmic signaling domain of CD3ζ and a co-stimulatory signaling domain of 4-1BB.

In some embodiments, the intracellular signaling domain of the CAR comprises a co-stimulatory signaling domain of CD28 and a co-stimulatory signaling domain of 4-1BB. In some embodiments, the intracellular signaling domain comprises a cytoplasmic signaling domain of CD3ζ, a co-stimulatory signaling domain of CD28, and a co-stimulatory signaling domain of 4-1BB. In some embodiments, the intracellular signaling domain comprises a polypeptide comprising from the N-terminus to the C-terminus: a co-stimulatory signaling domain of CD28, a co-stimulatory signaling domain of 4-1BB, and a cytoplasmic signaling domain of CD3ζ.

In some embodiments, the CAR comprises a polypeptide comprising from the N-terminus to the C-terminus: a CD8 leader, an extracellular binding domain, a CD8 hinge, a CD8 transmembrane, a 4-BB intracellular co-stimulatory domain, and a CD3ζ intracellular signaling domain.

In some embodiments, the CAR is a chimeric signaling domain (“CMSD”)-containing chimeric antigen receptor, wherein the CMSD comprises ITAMs (also referred to herein as “CMSD ITAMs”) and optional linkers (also referred to herein as “CMSD linkers”) arranged in a configuration that is different from any of the naturally occurring ITAM-containing parent molecules. For example, in some embodiments, the CMSD comprises two or more ITAMs directly linked to each other. In some embodiments, the CMSD comprises ITAMs connected by one or more “heterologous linkers”, namely, linker sequences which are either not derived from an ITAM-containing parent molecule (e.g., G/S linkers), or derive from an ITAM-containing parent molecule that is different from the ITAM-containing parent molecule from which one or more of the CMSD ITAMs are derived from. In some embodiments, the CMSD comprises two or more (such as 2, 3, 4, or more) identical ITAMs. In some embodiments, at least two of the CMSD ITAMs are different from each other. In some embodiments, at least one of the CMSD ITAMs is not derived from CD3ζ. In some embodiments, at least one of the CMSD ITAMs is not ITAM1 or ITAM2 of CD3ζ. In some embodiments, the CMSD does not comprise CD3ζ ITAM1 and/or CD3ζ ITAM2. In some embodiments, at least one of the CMSD ITAMs is CD3ζ ITAM3. In some embodiments, the CMSD does not comprise any ITAMs from CD3ζ. In some embodiments, at least two of the CMSD ITAMs are derived from the same ITAM-containing parent molecule. In some embodiments, the CMSD comprises two or more (such as 2, 3, 4, or more) ITAMs, wherein at least two of the CMSD ITAMs are each derived from a different ITAM-containing parent molecule. In some embodiments, at least one of the CMSD ITAMs is derived from an ITAM-containing parent molecule selected from the group consisting of: CD3ε, CD3δ, CD3γ, Igα (CD79a), Igβ (CD79b), FcεRIβ, FcεRIγ, DAP12, CNAIP/NFAM1, STAM-1, STAM-2, and Moesin.

In some embodiments, the CAR comprises a polypeptide comprising from the N-terminus to the C-terminus: a CD8 leader, an extracellular binding domain, a CD8 hinge, a CD8 transmembrane, a 4-1BB intracellular co-stimulatory domain, and one or more ITAM sequences.

In some embodiments, the targeting domain of the CAR is an antibody or an antibody fragment, such as an scFv, a Fv, a Fab, a (Fab′)₂, a single domain antibody (sdAb) such as a V_{H}H domain. In some embodiments, the targeting domain of the CAR is a ligand or an extracellular portion of a receptor that specifically binds to a tumor antigen. In some embodiments, the one or more targeting domains of the CAR specifically bind to a single tumor antigen. In some embodiments, the CAR is a bispecific or multispecific CAR with targeting domains that bind two or more tumor antigens. In some embodiments, the tumor antigen is selected from the group consisting of CD19, BCMA, NY-ESO-1, VEGFR2, MAGE-A3, CD20, CD22, CD33, CD38, CEA, EGFR (such as EGFRvII), GD2, HER2, IGF1R, mesothelin, PSMA, ROR1, WT1 and other tumor antigens with clinical significance, and combinations thereof.

In some embodiments, the CAR is an anti-CD19 CAR. In some embodiments, the CAR comprises an anti-CD19 scFv. In some embodiments, the anti-CD19 scFv is derived from an anti-CD19 antibody such as FMC63. In some embodiments, the CAR is an anti-CD19 CAR comprising the amino acid sequence of SEQ ID NO: 21.

SEQ ID NO: 21 (CTL-019 CAR; CD8α SP-CD19 scFv-CD8α hinge-CD8α TM-4-1BB-CD3ζ amino acid sequence; CD8α SP is italicized, CD8α hinge is squared, CD8α TM is italicized, 4-1BB cytoplasmic is underlined, CD3amino is bolded)

MALPVTALLLPLALLLHAARPDIQMTQTTSSLSASLGDRVTISCRASQD
ISKYLNWYQQKPDGTVKLLIYHTSRLHSGVPSRFSGSGSGTDYSLTISN
LEQEDIATYFCQQGNTLPYTFGGGTKLEITGGGGSGGGGSGGGGSEVKL
QESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYG

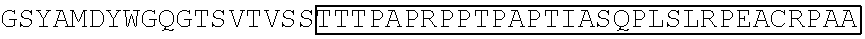

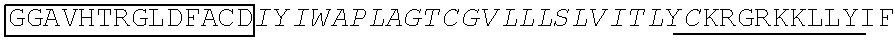

KQPFMRPVQTTQEEDGCSCRFPEEEEGGCELRVKFSRSADAPAYKQGQN
QLYNELNLGRREEYDVLDKRRGRDPEMGGKPRRKNPQEGLYNELQKDKM
AEAYSEIGMKGERRRGKGHDGLYQGLSTATKDTYDALHMQALPPR

In some embodiments, the CAR is an anti-BCMA CAR. A wide variety of antigen binding domain sequences can be used as the targeting domains of the CAR. See, e.g., WO2017/025038, which is incorporated herein in its entirety. In some embodiments, the CAR comprises an anti-BCMA scFv. In some embodiments, the CAR comprises an anti-BCMA sdAb, such as V_{H}H.

In some embodiments, the transmembrane domain of the CAR comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, IL-7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, TTGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In some embodiments, the transmembrane domain of the CAR is a CD4, CD3, CD8α, or CD28 transmembrane domain. In some embodiments, the transmembrane domain of the CAR comprises a transmembrane domain of CD8a.

In some embodiments, the extracellular domain is connected to the transmembrane domain by a hinge region. In one embodiment, the hinge region comprises the hinge region of CD8α.

In some embodiments, the CAR comprises a signal peptide, such as a CD8αSP.

Any CAR known in the art or developed by the inventors, including the CARs described in PCT/CN2017/096938 and PCT/CN2016/094408 (the contents of which are incorporated herein by reference in their entireties), may be used in the methods described herein. Exemplary structures of CARs are shown in FIGS. 15A-15D of PCT/CN2017/096938.

In some embodiments, the engineered receptor is a recombinant T-cell receptor. In some embodiments, the recombinant TCR is specific for a tumor antigen. In some embodiments, the tumor antigen is selected from the group consisting of CD19, BCMA, NY-ESO-1, VEGFR2, MAGE-A3, VEGFR2, MAGE-A3, CD20, CD22, CD33, CD38, CEA, EGFR (such as EGFRvIII), GD2, HER2, IGF1R, mesothelin, PSMA, ROR1, WT1 and other tumor antigens with clinical significance. In some embodiments, the tumor antigen is derived from an intracellular protein of tumor cells. Many TCRs specific for tumor antigens (including tumor-associated antigens) have been described, including, for example, NY-ESO-1 cancer-testis antigen, the p53 tumor suppressor antigens, TCRs for tumor antigens in melanoma (e.g., MARTI, gp 100), leukemia (e.g., WT1, minor histocompatibility antigens), and breast cancer (HER2, NY-BR1, for example). Any of the TCRs known in the art may be used in the present application. In some embodiments, the TCR has an enhanced affinity to the tumor antigen. Exemplary TCRs and methods for introducing the TCRs to immune cells have been described, for example, in U.S. Pat. No. 5,830,755, and Kessels et al. Immunotherapy through TCR gene transfer. Nat. Immunol. 2, 957-961 (2001). In some embodiments, the modified therapeutic cell is a TCR-T cell.

The TCR receptor complex is an octomeric complex formed by variable TCR receptor α and β chains (γ and δ chains on case of γδ T cells) with three dimeric signaling modules CD3δ/ε, CD3γ/ε and CD247 (T-cell surface glycoprotein CD3 zeta chain) ζ/ζ or ζ/η. Ionizable residues in the transmembrane domain of each subunit form a polar network of interactions that hold the complex together. TCR complex has the function of activating signaling cascades in T cells.

In some embodiments, the engineered receptor is an engineered TCR comprising one or more T-cell receptor (TCR) fusion proteins (TFPs). Exemplary TFPs have been described, for example, in US20170166622A1, which is incorporated herein by reference. In some embodiments, the TFP comprises an extracellular domain of a TCR subunit that comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications. In some embodiments, the TFP comprises a transmembrane domain that comprises a transmembrane domain of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications. In some embodiments, the TFP comprises a transmembrane domain that comprises a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

In some embodiments, the TFP comprising a TCR subunit comprising at least a portion of a TCR extracellular domain, and a TCR intracellular domain comprising a stimulatory domain from an intracellular signaling domain of CD3 epsilon; and an antigen binding domain, wherein the TCR subunit and the antigen binding domain are operatively linked, and wherein the TFP incorporates into a TCR when expressed in a T cell.

In some embodiments, the TFP comprises a TCR subunit comprising at least a portion of a TCR extracellular domain, and a TCR intracellular domain comprising a stimulatory domain from an intracellular signaling domain of CD3 gamma; and an antigen binding domain wherein the TCR subunit and the antigen binding domain are operatively linked, and wherein the TFP incorporates into a TCR when expressed in a T cell.

In some embodiments, the TFP comprises a TCR subunit comprising at least a portion of a TCR extracellular domain, and a TCR intracellular domain comprising a stimulatory domain from an intracellular signaling domain of CD3 delta; and an antigen binding domain, wherein the TCR subunit and the antigen binding domain are operatively linked, and wherein the TFP incorporates into a TCR when expressed in a T cell.

In some embodiments, the TFP comprises a TCR subunit comprising at least a portion of a TCR extracellular domain, and a TCR intracellular domain comprising a stimulatory domain from an intracellular signaling domain of TCR alpha; and an antigen binding domain wherein the TCR subunit and the antigen binding domain are operatively linked, and wherein the TFP incorporates into a TCR when expressed in a T cell.

In some embodiments, the TFP comprises a TCR subunit comprising at least a portion of a TCR extracellular domain, and a TCR intracellular domain comprising a stimulatory domain from an intracellular signaling domain of TCR beta; and an antigen binding domain wherein the TCR subunit and the antigen binding domain are operatively linked, and wherein the TFP incorporates into a TCR when expressed in a T cell.

In some embodiments, the engineered receptor is a T-cell antigen coupler (TAC) receptor. Exemplary TAC receptors have been described, for example, in US20160368964A1, which is incorporated herein by reference. In some embodiments, the TAC comprises a targeting domain, a TCR-binding domain that specifically binds a protein associated with the TCR complex, and a T-cell receptor signaling domain. In some embodiments, the targeting domain is an antibody fragment, such as scFv or V_{H}H, which specifically binds to a tumor antigen. In some embodiments, the targeting domain is a designed Ankyrin repeat (DARPin) polypeptide. In some embodiments, the tumor antigen is selected from the group consisting of CD19, BCMA, NY-ESO-1, VEGFR2, MAGE-A3, VEGFR2, MAGE-A3, CD20, CD22, CD33, CD38, CEA, EGFR (such as EGFRvH), GD2, HER2, IGF1R, mesothelin, PSMA, ROR1, WT1 and other tumor antigens with clinical significance. In some embodiments, the protein associated with the TCR complex is CD3, such as CD3ε. In some embodiments, the TCR-binding domain is a single chain antibody, such as scFv, or a V_{H}H. In some embodiments, the TCR-binding domain is derived from UCHT1. In some embodiments, the TAC receptor comprises a cytosolic domain and a transmembrane domain. In some embodiments, the T-cell receptor signaling domain comprises a cytosolic domain derived from a TCR co-receptor. Exemplary TCR co-receptors include, but are not limited to, CD4, CD8, CD28, CD45, CD4, CD5, CD9, CD16, CD22, CD33, CD37, CD64, CD80, CD86, CD134, CD137 and CD 154. In some embodiments, the TAC receptor comprises a transmembrane domain and a cytosolic domain derived from CD4. In some embodiments, the TAC receptor comprises a transmembrane domain and a cytosolic domain derived from CD8 (such as CD8a).

T cell co-receptors are expressed as membrane proteins on T cells. They can provide stabilization of the TCR: peptide: MHC complex and facilitate signal transduction. The two subtypes of T cell co-receptor, CD4 and CD8, display strong specificity for particular MHC classes. The CD4 co-receptor can only stabilize TCR: MHC H complexes while the CD8 co-receptor can only stabilize the TCR: MHC I complex. The differential expression of CD4 and CD8 on different T cell types results in distinct T cell functional subpopulations. CD8+ T cells are cytotoxic T cells.

CD4 is a glycoprotein expressed on the surface of immune cells such as T helper cells, monocytes, macrophages, and dendritic cells. CD4 has four immunoglobulin domains (D₁ to D₄) exposed on the extracellular cell surface. CD4 contains a special sequence of amino acids on its short cytoplasmic/intracellular tail, which allow CD4 tail to recruit and interact with the tyrosine kinase Lck. When the TCR complex and CD4 each bind to distinct regions of the MHC II molecule, the close proximity between the TCR complex and CD4 allows Lck bound to the cytoplasmic tail of CD4 to tyrosine-phosphorylate the Immunoreceptor Tyrosine Activation Motifs (ITAM) on the cytoplasmic domains of CD3, thus amplifying TCR generated signal.

CD8 is a glycoprotein of either a homodimer composed of two a chains (less common), or a heterodimer composed of one α and one β chain (more common), each comprising an immunoglobulin variable (IgV)-like extracellular domain connected to the membrane by a thin stalk, and an intracellular tail. CD8 is predominantly expressed on the surface of cytotoxic T cells, but can also be found on natural killer cells, cortical thymocytes, and dendritic cells. The CD8 cytoplasmic tail interacts with Lck, which phosphorylates the cytoplasmic CD3 and (-chains of the TCR complex once TCR binds its specific antigen. Tyrosine-phosphorylation on the cytoplasmic CD3 and ζ-chains initiates a cascade of phosphorylation, eventually leading to gene transcription.

In some embodiments, the modified therapeutic cell expresses more than one engineered receptor, such as any combination of CAR, recombinant TCRs, TAC receptors and TFPs.

In some embodiments, the engineered receptor (such as CAR, TCR, TAC or TFP) expressed by the modified therapeutic cell targets one or more tumor antigens. Tumor antigens are proteins that are produced by tumor cells that can elicit an immune response, particularly T-cell mediated immune responses. The selection of the targeted antigen of the invention will depend on the particular type of cancer to be treated. Exemplary tumor antigens include, for example, a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In some embodiments, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA). In B-cell lymphoma, the tumor-specific idiotype immunoglobulin constitutes a truly tumor-specific immunoglobulin antigen that is unique to the individual tumor. B cell differentiation antigens such as CD19, CD20 and CD37 are other candidates for target antigens in B-cell lymphoma.

In some embodiments, the tumor antigen is a tumor-specific antigen (TSA) or a tumor-associated antigen (TAA). A TSA is unique to tumor cells and does not occur on other cells in the body. A TAA is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include the following: Differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, pl80erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

### C. Nucleic Acids

The modified therapeutic cells described herein comprise one or more nucleic acids comprising heterologous nucleic acid sequence(s) encoding any one of the agents for reducing MHC expression and/or engineered receptors described herein. In some embodiments, the nucleic acid is a DNA. In some embodiments, the nucleic acid is a RNA. In some embodiments, the nucleic acid is linear. In some embodiments, the nucleic acid is circular.

The heterologous nucleic acid sequence(s) may be operably linked to one or more regulatory sequences. Exemplary regulatory sequences that control the transcription and/or translation of a coding sequence are known in the art and may include, but not limited to, a promoter, additional elements for proper initiation, regulation and/or termination of transcription (e.g. polyA transcription termination sequences), mRNA transport (e.g. nuclear localization signal sequences), processing (e.g. splicing signals), stability (e.g. introns and non-coding 5′ and 3′ sequences), translation (e.g. an initiator Met, tripartite leader sequences, IRES ribosome binding sites, signal peptides, etc.), and insertion site for introducing an insert into the viral vector. In some embodiments, the regulatory sequence is a promoter, a transcriptional enhancer and/or a sequence that allows for proper expression of the agent and/or the engineered receptor.

The term “regulatory sequence” or “control sequence” refers to a DNA sequence that affects the expression of a coding sequence to which it is operably linked. The nature of such regulatory sequences differs depending upon the host organism. In prokaryotes, regulatory sequences generally include promoters, ribosomal binding sites, and terminators. In eukaryotes, regulatory sequences include promoters, terminators and, in some instances, enhancers, transactivators or transcription factors.

The term “operably linked” refers to a juxtaposition wherein the components so described are in a relationship permitting them to function in their intended manner. A regulatory sequence “operably linked” to a coding sequence is ligated in such a way that expression of the coding sequence is achieved under conditions compatible with the regulatory sequences.

As used herein, a “promoter” or a “promoter region” refers to a segment of DNA or RNA that controls transcription of the DNA or RNA to which it is operatively linked. The promoter region includes specific sequences that are involved in RNA polymerase recognition, binding and transcription initiation. In addition, the promoter includes sequences that modulate recognition, binding and transcription initiation activity of RNA polymerase (i.e., binding of one or more transcription factors). These sequences can be cis acting or can be responsive to trans acting factors. Promoters, depending upon the nature of the regulation, can be constitutive or regulated. Regulated promoters can be inducible or environmentally responsive (e.g. respond to cues such as pH, anaerobic conditions, osmoticum, temperature, light, or cell density). Many such promoter sequences are known in the art. See, for example, U.S. Pat. Nos. 4,980,285; 5,631,150; 5,707,928; 5,759,828; 5,888,783; 5,919,670, and, Sambrook, et al., Molecular Cloning: A Laboratory Manual, 2nd Ed., Cold Spring Harbor Press (1989).

In some embodiments, the promoter is an endogenous promoter. For example, a nucleic acid sequencing encoding the engineered receptor may be knocked-in to the genome of a modified immune cell downstream of an endogenous promoter using any methods known in the art, such as CRISPR/Cas9 method. In some embodiments, the endogenous promoter is a promoter for an abundant protein, such as beta-actin. In some embodiments, the endogenous promoter is an inducible promoter, for example, inducible by an endogenous activation signal of the modified therapeutic cell (e.g., modified immune cell). In some embodiments, wherein the modified therapeutic cell is a T cell, the promoter is a T cell activation-dependent promoter (such as an IL-2 promoter, an NFAT promoter, or an NFκB promoter). In some embodiments, the promoter is a heterologous promoter.

Varieties of promoters have been explored for gene expression in mammalian cells, and any of the promoters known in the art may be used in the present invention. Promoters may be roughly categorized as constitutive promoters or regulated promoters, such as inducible promoters. In some embodiments, the heterologous nucleic acid sequence encoding the engineered receptor is operably linked to a constitutive promoter. In some embodiments, the heterologous nucleic acid sequence encoding the engineered receptor is operably linked to an inducible promoter.

Constitutive promoters allow heterologous genes (also referred to as transgenes) to be expressed constitutively in the host cells. Exemplary constitutive promoters contemplated herein include, but are not limited to, Cytomegalovirus (CMV) promoters, human elongation factors-1alpha (hEF1α), ubiquitin C promoter (UbiC), phosphoglycerokinase promoter (PGK), simian virus 40 early promoter (SV40), and chicken β-Actin promoter coupled with CMV early enhancer (CAGG). The efficiencies of such constitutive promoters on driving transgene expression have been widely compared in a huge number of studies. In some embodiments, the promoter is a hEF1α promoter.

In some embodiments, the promoter is an inducible promoter. Inducible promoters belong to the category of regulated promoters. The inducible promoter can be induced by one or more conditions, such as a physical condition, microenvironment of the modified therapeutic cell (e.g., modified immune cell), or the physiological state of the modified therapeutic cell (e.g., modified immune cell), an inducer (i.e., an inducing agent), or a combination thereof. In some embodiments, the inducing condition does not induce the expression of endogenous genes in the modified therapeutic cell (e.g., modified immune cell), and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the modified therapeutic cell (e.g., modified immune cell).

In some embodiments, the promoter is inducible by an inducer. In some embodiments, the inducer is a small molecule, such as a chemical compound. In some embodiments, the small molecule is selected from the group consisting of doxycycline, tetracycline, alcohol, metal, or steroids. Chemically-induced promoters have been most widely explored. Such promoters includes promoters whose transcriptional activity is regulated by the presence or absence of a small molecule chemical, such as doxycycline, tetracycline, alcohol, steroids, metal and other compounds. Doxycycline-inducible system with reverse tetracycline-controlled transactivator (rtTA) and tetracycline-responsive element promoter (TRE) is the most established system at present. WO9429442 describes the tight control of gene expression in eukaryotic cells by tetracycline responsive promoters. WO9601313 discloses tetracycline-regulated transcriptional modulators. Additionally, Tet technology, such as the Tet-on system, has described, for example, on the website of TetSystems.com. Any of the known chemically regulated promoters may be used to drive expression of the therapeutic protein in the present application.

In some embodiments, the inducer is a polypeptide, such as a growth factor, a hormone, or a ligand to a cell surface receptor, for example, a polypeptide that specifically binds a tumor antigen. Many polypeptide inducers are also known in the art, and they may be suitable for use in the present invention. For example, ecdysone receptor-based gene switches, progesterone receptor-based gene switches, and estrogen receptor based gene switches belong to gene switches employing steroid receptor derived transactivators (WO9637609 and WO9738117 etc.).

In some embodiments, the inducer comprises both a small molecule component and one or more polypeptides. For example, inducible promoters that dependent on dimerization of polypeptides are known in the art, and may be suitable for use in the present invention. The first small molecule CID system, developed in 1993, used FK1012, a derivative of the drug FK506, to induce homo-dimerization of FKBP. By employing similar strategies, Wu et al successfully make the CAR-T cells titratable through an ON-switch manner by using Rapalog/FKPB-FRB* and Gibberelline/GID1-GAI dimerization dependent gene switch (C.-Y. Wu et al., Science 350, aab4077 (2015)). Other dimerization dependent switch systems include Coumermycin/GyrB-GyrB (Nature 383 (6596): 178-81), and HaXS/Snap-tag-HaloTag (Chemistry and Biology 20 (4): 549-57).

In some embodiments, the promoter is a light-inducible promoter, and the inducing condition is light. Light inducible promoters for regulating gene expression in mammalian cells are also well-known in the art (see, for example, Science 332, 1565-1568 (2011); Nat. Methods 9, 266-269 (2012); Nature 500: 472-476 (2013); Nature Neuroscience 18:1202-1212 (2015)). Such gene regulation systems can be roughly divided into two categories based on their regulations of (1) DNA binding or (2) recruitment of a transcriptional activation domain to a DNA bound protein. For instance, synthetic mammalian blue light controlled transcription system based on melanopsin, which, in response to blue light (480 nm), triggers an intracellular calcium increase that result in calcineurin-mediated mobilization of NFAT, were developed and tested in mammalian cells. More recently, Motta-Mena et al described a new inducible gene expression system developed from naturally occurring EL222 transcription factor that confers high-level, blue light-sensitive control of transcriptional initiation in human cell lines and zebrafish embryos (Nat. Chem. Biol. 10(3):196-202 (2014)). Additionally, the red light induced interaction of photoreceptor phytochrome B (PhyB) and phytochrome-interacting factor 6 (PIF6) of Arabidopsis thaliana was exploited for a red light triggered gene expression regulation. Furthermore, ultraviolet B (UVB)-inducible gene expression system were also developed and proven to be efficient in target gene transcription in mammalian cells (Chapter 25 of Gene and Cell Therapy: Therapeutic Mechanisms and Strategies, Fourth Edition CRC Press, Jan. 20, 2015). Any of the light-inducible promoters described herein may be used to drive expression of the therapeutic protein in the present invention.

In some embodiments, the promoter is a light-inducible promoter that is induced by a combination of a light-inducible molecule, and light. For example, a light-cleavable photocaged group on a chemical inducer keeps the inducer inactive, unless the photocaged group is removed through irradiation or by other means. Such light-inducible molecules include small molecule compounds, oligonucleotides, and proteins. For example, caged ecdysone, caged IPTG for use with the lac operon, caged toyocamycin for ribozyme-mediated gene expression, caged doxycycline for use with the Tet-on system, and caged Rapalog for light mediated FKBP/FRB dimerization have been developed (see, for example, Curr Opin Chem Biol. 16(3-4): 292-299 (2012)).

In some embodiments, the promoter is a radiation-inducible promoter, and the inducing condition is radiation, such as ionizing radiation. Radiation inducible promoters are also known in the art to control transgene expression. Alteration of gene expression occurs upon irradiation of cells. For example, a group of genes known as “immediate early genes” can react promptly upon ionizing radiation. Exemplary immediate early genes include, but are not limited to, Erg-1, p21/WAF-1, GADD45alpha, t-PA, c-Fos, c-Jun, NF-kappaB, and API. The immediate early genes comprise radiation responsive sequences in their promoter regions. Consensus sequences have been found in the Erg-1 promoter, and are referred to as serum response elements or known as CArG elements. Combinations of radiation induced promoters and transgenes have been intensively studied and proven to be efficient with therapeutic benefits. See, for example, Cancer Biol Ther. 6(7):1005-12 (2007) and Chapter 25 of Gene and Cell Therapy: Therapeutic Mechanisms and Strategies, Fourth Edition CRC Press, Jan. 20, 2015.

In some embodiments, the promoter is a heat inducible promoter, and the inducing condition is heat. Heat inducible promoters driving transgene expression have also been widely studied in the art. Heat shock or stress protein (HSP) including Hsp90, Hsp70, Hsp60, Hsp40, Hsp10 etc. plays important roles in protecting cells under heat or other physical and chemical stresses. Several heat inducible promoters including heat-shock protein (HSP) promoters and growth arrest and DNA damage (GADD) 153 promoters have been attempted in pre-clinical studies. The promoter of human hsp70B gene, which was first described in 1985 appears to be one of the most highly-efficient heat inducible promoters. Huang et al reported that after introduction of hsp70B-EGFP, hsp70B-TNFalpha and hsp70B-IL12 coding sequences, tumor cells expressed extremely high transgene expression upon heat treatment, while in the absence of heat treatment, the expression of transgenes were not detected. Tumor growth was delayed significantly in the IL12 transgene plus heat-treated group of mice in vivo (Cancer Res. 60:3435 (2000)). Another group of scientists linked the HSV-tk suicide gene to hsp70B promoter and test the system in nude mice bearing mouse breast cancer. Mice whose tumor had been administered the hsp70B-HSVtk coding sequence and heat-treated showed tumor regression and a significant survival rate as compared to no heat treatment controls (Hum. Gene Ther. 11:2453 (2000)). Additional heat inducible promoters known in the art can be found in, for example, Chapter 25 of Gene and Cell Therapy: Therapeutic Mechanisms and Strategies, Fourth Edition CRC Press, Jan. 20, 2015. Any of the heat-inducible promoters discussed herein may be used to drive the expression of the therapeutic protein of the present invention.

In some embodiments, the promoter is inducible by a redox state. Exemplary promoters that are inducible by redox state include inducible promoter and hypoxia inducible promoters. For instance, Post D E et al developed hypoxia-inducible factor (HIF) responsive promoter, which specifically and strongly induce transgene expression in HIF-active tumor cells (Gene Ther. 8: 1801-1807 (2001); Cancer Res. 67: 6872-68δ1 (2007)).

In some embodiments, the promoter is inducible by the physiological state, such as an endogenous activation signal, of the modified therapeutic cell (e.g., modified immune cell). In some embodiments, wherein the modified therapeutic cell is a T cell, the promoter is a T cell activation-dependent promoter, which is inducible by the endogenous activation signal of the modified T cell. In some embodiments, the modified T cell is activated by an inducer, such as phorbol myristate acetate (PMA), ionomycin, or phytohaemagglutinin. In some embodiments, the modified T cell is activated by recognition of a tumor antigen on the tumor cells via the engineered receptor (such as CAR, TCR or TAC). In some embodiments, the T cell activation-dependent promoter is an IL-2 promoter. In some embodiments, the T cell activation-dependent promoter is an NFAT promoter. In some embodiments, the T cell activation-dependent promoter is a NFκB promoter.

The heterologous nucleic acid sequences(s) described herein can be present in a heterologous gene expression cassette, which comprises one or more protein-coding sequences and optionally one or more promoters. In some embodiments, the heterologous gene expression cassette comprises a single protein-coding sequence. In some embodiments, the heterologous gene expression cassette comprises two or more protein-coding sequences driven by a single promoter (i.e., polycistronic). In some embodiments, the heterologous gene expression cassette further comprises one or more regulatory sequences (such as 5′UTR, 3′UTR, enhancer sequence, IRES, transcription termination sequence), recombination sites, one or more selection markers (such as antibiotic resistance gene, reporter gene, etc.), signal sequence, or combinations thereof. In some embodiments, a first heterologous nucleic acid sequence encoding the sICP47 or functional variant thereof is fused to a second heterologous nucleic acid sequence encoding an agonist agent of an NKG2A inhibitory receptor via a third nucleic acid sequence encoding a self-cleavable linker, such as P2A, T2A, E2A, or F2A peptide. In some embodiments, the self-cleavable linker is a P2A peptide comprising the amino acid sequence of SEQ ID NO: 23.

In some embodiments, the heterologous nucleic acid sequence(s) are present in a vector. In some embodiments, the modified therapeutic cell comprises a vector comprising a first heterologous nucleic acid sequence encoding a sICP47 protein or functional variant thereof. In some embodiments, the modified therapeutic cell comprises a vector comprising a first heterologous nucleic acid sequence encoding a sICP47 protein or functional variant thereof and a second heterologous nucleic acid sequence encoding an engineered receptor (e.g., CAR). In some embodiments, the modified therapeutic cell comprises a vector comprising a first heterologous nucleic acid sequence encoding a sICP47 protein or functional variant thereof and a second heterologous nucleic acid sequence encoding an agonist agent of an NK cell inhibitory receptor (e.g., membrane-bound anti-NKG2A antibody). In some embodiments, the modified therapeutic cell comprises a vector comprising a first heterologous nucleic acid sequence encoding a sICP47 protein or functional variant thereof, a second heterologous nucleic acid sequence encoding an agonist agent of an NK cell inhibitory receptor (e.g., membrane-bound anti-NKG2A antibody), and a third heterologous nucleic acid sequence encoding an engineered receptor (e.g., CAR). In some embodiments, the vector comprises an EGFP comprising the amino acid sequence of SEQ ID NO: 22.

In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the therapeutic cell (e.g., modified immune cell) in vitro or ex vivo. A number of retroviral systems are known in the art. In some embodiments, adenovirus vectors are used. In some embodiments, lentivirus vectors are used. In some embodiments, self-inactivating lentiviral vectors are used. For example, self-inactivating lentiviral vectors can be packaged with protocols known in the art. The resulting lentiviral vectors can be used to transduce a mammalian cell (such as human T cells) using methods known in the art.

In some embodiments, the vector is a non-viral vector, such as a plasmid, or an episomal expression vector.

In some embodiments, the vector is an expression vector. “Expression vector” is a construct that can be used to transform a selected host and provides for expression of a coding sequence in the selected host. Expression vectors can for instance be cloning vectors, binary vectors or integrating vectors. Expression comprises transcription of the nucleic acid molecule preferably into a translatable mRNA. Regulatory elements ensuring expression in eukaryotic cells are well known to those skilled in the art. In the case of eukaryotic cells they comprise normally promoters ensuring initiation of transcription and optionally poly-A signals ensuring termination of transcription and stabilization of the transcript. Examples of regulatory elements permitting expression in eukaryotic host cells are AOX1 or GAL1 promoter in yeast or the CMV-, SV40-, RSV-promoter (Rous sarcoma virus), CMV-enhancer, SV40-enhancer or a globin intron in mammalian and other animal cells. Furthermore, depending on the expression system used leader sequences capable of directing the polypeptide to a cellular compartment or secreting it into the medium may be added to the coding sequence of the recited nucleic acid sequence and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the nucleic acid sequence can encode a fusion protein including an N-terminal identification peptide imparting desired characteristics, e.g., stabilization or simplified purification of expressed recombinant product. Suitable expression vectors are known in the art such as Okayama-Berg cDNA expression vector pcDV1 (Pharmacia), pEF-Neo, pCDM8, pRc/CMV, pcDNA1, pcDNA3 (Invitrogen), pEF-DHFR and pEF-ADA, (Raum et al., Cancer Immunol Immunother (2001) 50(3), 141-150) or pSPORT1 (GIBCO BRL).

### III. Methods of Treatment

One aspect of the present application relates to methods of treating a disease or condition (such as cancer) in an individual, comprising administering to the individual an effective amount of any one of the modified therapeutic cells described herein. The present application contemplates modified therapeutic cells that can be administered either alone or in any combination with another therapy, and in at least some aspects, together with a pharmaceutically acceptable carrier or excipient. In some embodiments, prior to administration, the modified therapeutic cells may be combined with suitable pharmaceutical carriers and excipients that are well known in the art.

In some embodiments, there is provided a method of treating a disease or condition (e.g., cancer) in an individual (e.g., human), comprising administering to the individual an effective amount of a pharmaceutical composition comprising a modified therapeutic cell (e.g., a modified immune cell) and a pharmaceutically acceptable carrier, wherein the modified therapeutic cell comprises a first heterologous nucleic acid sequence encoding a sICP47 protein or a functional variant thereof. In some embodiments, the first heterologous nucleic acid sequence encodes a naturally occurring sICP47 protein. In some embodiments, the first heterologous nucleic acid sequence encodes an engineered sICP47 protein comprising an active domain of a naturally occurring sICP47 protein. In some embodiments, the naturally occurring sICP47 protein is from a herpesvirus selected from the group consisting of Simian Agent 8 (SAB), Cercopithecine herpesvirus 16 (CeHV-16) and Cercopithecine herpesvirus 1 (CeHV-1). In some embodiments, the first heterologous nucleic acid sequence encodes a sICP47 of SA8 or a functional variant thereof. In some embodiments, the first heterologous nucleic acid sequence encodes a sICP47 of CeHV-16 or a functional variant thereof. In some embodiments, the sICP47 protein or functional variant thereof comprises an amino acid sequence having at least about 80% (e.g., at least about any one of 85%, 90%, 92%, 95%, 98%, 99%, or more) sequence identity to an amino acid sequence selected from the group consisting of SEQ ID NOs: 1-3. In some embodiments, the sICP47 protein or functional variant thereof comprises an amino acid sequence having at least about 80% (e.g., at least about any one of 85%, 90%, 92%, 95%, 98%, 99%, or more) sequence identity to an amino acid sequence selected from the group consisting of SEQ ID NOs: 4-6 and 24-25. In some embodiments, the therapeutic cell is an immune cell selected from the group consisting of cytotoxic T cell, helper T cell, NK cell, NK-T cell, as T cell, γδ T cell, tumor-infiltrating T cell, dendritic cell (DC)-activated T cell, and peripheral blood mononuclear cell (PBMC). In some embodiments, the modified immune cell is a T cell. In some embodiments, the modified immune cell is a T cell. In some embodiments, the modified immune cell is a γδ T cell comprising, such as a γ9δ2 T cell, a δ1 T cell, or a δ3 T cell. In some embodiments, the modified therapeutic cell further comprises a heterologous nucleic acid sequence encoding an agonist agent of an NK cell inhibitory receptor. In some embodiments, the agonist agent is an antibody or antigen-binding fragment thereof that specifically binds to the NK cell inhibitory receptor. In some embodiments, the agonist agent is a membrane-bound antibody that specifically binds NKG2A. In some embodiments, the agonist agent is a ligand of the NK cell inhibitory receptor. In some embodiments, the agonist agent comprises a VH comprising a HC-CDR1 comprising SEQ ID NO: 7, a HC-CDR2 comprising SEQ ID NO: 8, a HC-CDR3 comprising SEQ ID NO: 9, and a VL comprising a LC-CDR1 comprising SEQ ID NO: 10, a LC-CDR2 comprising SEQ ID NO: 11, and a LC-CDR3 comprising SEQ ID NO: 12. In some embodiments, the agonist agent comprises a VH comprising SEQ ID NO: 13 and a VL comprising SEQ ID NO: 14. In some embodiments, the agonist agent is a polypeptide comprising from the N-terminus to the C-terminus a single-chain variable fragment (scFv) that specifically binds to NKG2A, a CD8α hinge sequence and a CD8α transmembrane sequence.

In some embodiments, there is provided a method of treating a disease or condition (e.g., cancer) in an individual (e.g., human), comprising administering to the individual an effective amount of a pharmaceutical composition comprising a modified immune cell or progenitor thereof and a pharmaceutically acceptable carrier, wherein the modified immune cell or progenitor thereof comprises a first heterologous nucleic acid sequence encoding a sICP47 protein or a functional variant thereof and a second heterologous nucleic acid sequence encoding an engineered receptor (e.g., CAR). In some embodiments, the first heterologous nucleic acid sequence encodes a naturally occurring sICP47 protein. In some embodiments, the first heterologous nucleic acid sequence encodes an engineered sICP47 protein comprising an active domain of a naturally occurring sICP47 protein. In some embodiments, the naturally occurring sICP47 protein is from a herpesvirus selected from the group consisting of Simian Agent 8 (SA8), Cercopithecine herpesvirus 16 (CeHV-16) and Cercopithecine herpesvirus 1 (CeHV-1). In some embodiments, the first heterologous nucleic acid sequence encodes a sICP47 of SA8 or a functional variant thereof. In some embodiments, the first heterologous nucleic acid sequence encodes a sICP47 of CeHV-16 or a functional variant thereof. In some embodiments, the sICP47 protein or functional variant thereof comprises an amino acid sequence having at least about 80% (e.g., at least about any one of 85%, 90%, 92%, 95%, 98%, 99%, or more) sequence identity to an amino acid sequence selected from the group consisting of SEQ ID NOs: 1-3. In some embodiments, the sICP47 protein or functional variant thereof comprises an amino acid sequence having at least about 80% (e.g., at least about any one of 85%, 90%, 92%, 95%, 98%, 99%, or more) sequence identity to an amino acid sequence selected from the group consisting of SEQ ID NOs: 4-6 and 24-25. In some embodiments, the engineered receptor is selected from the group consisting of a chimeric antigen receptor (CAR), a recombinant T cell receptor (TCR), a T-cell antigen coupler (TAC) receptor and a TCR fusion protein (TFP). In some embodiments, the engineered receptor targets a tumor antigen selected from the group consisting of CD19, BCMA, NY-ESO-1, VEGFR2, MAGE-A3, CD20, CD22, CD33, CD38, CEA, EGFR, GD2, HER2, IGF1R, mesothelin, PSMA, ROR1, WT1 and combinations thereof. In some embodiments, the modified immune cell or progenitor thereof is allogeneic. In some embodiments, the immune cell is selected from the group consisting of cytotoxic T cell, helper T cell, NK cell, NK-T cell, αβ T cell, γδ T cell, tumor-infiltrating T cell, dendritic cell (DC)-activated T cell, and peripheral blood mononuclear cell (PBMC). In some embodiments, the modified immune cell is a T cell. In some embodiments, the modified immune cell is a γδ T cell. In some embodiments, the modified immune cell is a γ9δ2 T cell. In some embodiments, the modified immune cell is a δ1 T cell. In some embodiments, the modified immune cell is a δ3 T cell. In some embodiments, the modified progenitor cell is HSC. In some embodiments, the modified immune cell further comprises a third heterologous nucleic acid sequence encoding an agonist agent of an NK cell inhibitory receptor. In some embodiments, the agonist agent is an antibody or antigen-binding fragment thereof that specifically binds to the NK cell inhibitory receptor. In some embodiments, the agonist agent is a membrane-bound antibody that specifically binds NKG2A. In some embodiments, the agonist agent is a ligand of the NK cell inhibitory receptor. In some embodiments, the agonist agent comprises a VH comprising a HC-CDR1 comprising SEQ ID NO: 7, a HC-CDR2 comprising SEQ ID NO: 8, a HC-CDR3 comprising SEQ ID NO: 9, and a VL comprising a LC-CDR1 comprising SEQ ID NO: 10, a LC-CDR2 comprising SEQ ID NO: 11, and a LC-CDR3 comprising SEQ ID NO: 12. In some embodiments, the agonist agent comprises a VH comprising SEQ ID NO: 13 and a VL comprising SEQ ID NO: 14. In some embodiments, the agonist agent is a polypeptide comprising from the N-terminus to the C-terminus a single-chain variable fragment (scFv) that specifically binds to NKG2A, a CD8α hinge sequence and a CD8α transmembrane sequence.

In some embodiments, there is provided a method of treating a disease or condition (e.g., cancer) in an individual (e.g., human), comprising administering to the individual an effective amount of a pharmaceutical composition comprising a CAR-T cell and a pharmaceutically acceptable carrier, wherein the CAR-T cell comprises a first heterologous nucleic acid sequence encoding a sICP47 protein or a functional variant thereof, and a second heterologous nucleic acid sequence encoding a CAR. In some embodiments, the first heterologous nucleic acid sequence encodes a naturally occurring sICP47 protein. In some embodiments, the first heterologous nucleic acid sequence encodes an engineered sICP47 protein comprising an active domain of a naturally occurring sICP47 protein. In some embodiments, the naturally occurring sICP47 protein is from a herpesvirus selected from the group consisting of Simian Agent 8 (SA8), Cercopithecine herpesvirus 16 (CeHV-16) and Cercopithecine herpesvirus 1 (CeHV-1). In some embodiments, the first heterologous nucleic acid sequence encodes a sICP47 of SA8 or a functional variant thereof. In some embodiments, the first heterologous nucleic acid sequence encodes a sICP47 of CeHV-16 or a functional variant thereof. In some embodiments, the sICP47 protein or functional variant thereof comprises an amino acid sequence having at least about 80% (e.g., at least about any one of 85%, 90%, 92%, 95%, 98%, 99%, or more) sequence identity to an amino acid sequence selected from the group consisting of SEQ ID NOs: 1-3. In some embodiments, the sICP47 protein or functional variant thereof comprises an amino acid sequence having at least about 80% (e.g., at least about any one of 85%, 90%, 92%, 95%, 98%, 99%, or more) sequence identity to an amino acid sequence selected from the group consisting of SEQ ID NOs: 4-6 and 24-25. In some embodiments, the CAR targets a tumor antigen selected from the group consisting of CD19, BCMA, NY-ESO-1, VEGFR2, MAGE-A3, CD20, CD22, CD33, CD38, CEA, EGFR, GD2, HER2, IGF1R, mesothelin, PSMA, ROR1, WT1 and combinations thereof. In some embodiments, the CAR is an anti-BCMA CAR. In some embodiments, the CAR is an anti-CD19 CAR. In some embodiments, the CAR-T cell is a γδ T cell. In some embodiments, the CAR-T cell is a γ9δ2 T cell. In some embodiments, the CAR-T cell is a δ1 T cell. In some embodiments, the CAR-T cell is a δ3 T cell. In some embodiments, the CAR-T cell further comprises a third heterologous nucleic acid sequence encoding an agonist agent of an NK cell inhibitory receptor. In some embodiments, the agonist agent is an antibody or antigen-binding fragment thereof that specifically binds to the NK cell inhibitory receptor. In some embodiments, the agonist agent is a membrane-bound antibody that specifically binds NKG2A. In some embodiments, the agonist agent is a ligand of the NK cell inhibitory receptor. In some embodiments, the agonist agent comprises a VH comprising a HC-CDR1 comprising SEQ ID NO: 7, a HC-CDR2 comprising SEQ ID NO: 8, a HC-CDR3 comprising SEQ ID NO: 9, and a VL comprising a LC-CDR1 comprising SEQ ID NO: 10, a LC-CDR2 comprising SEQ ID NO: 11, and a LC-CDR3 comprising SEQ ID NO: 12. In some embodiments, the agonist agent comprises a VH comprising SEQ ID NO: 13 and a VL comprising SEQ ID NO: 14. In some embodiments, the agonist agent is a polypeptide comprising from the N-terminus to the C-terminus a single-chain variable fragment (scFv) that specifically binds to NKG2A, a CD8α hinge sequence and a CD8α transmembrane sequence.

The methods described herein are suitable for treating diseases or conditions such as cancer, autoimmune diseases, or infectious diseases. The methods described herein can be used to treat a variety of cancers, including both solid cancer and liquid cancer. In some embodiments, the cancer is selected from the group consisting of leukemia, lymphoma, melanoma, breast cancer, lung cancer, liver cancer, gastric cancer, colon cancer, bone cancer, brain cancer, pancreatic cancer, and ovarian cancer. The methods are applicable to cancers of all stages, including early stage cancer, non-metastatic cancer, primary cancer, advanced cancer, locally advanced cancer, metastatic cancer, or cancer in remission. The methods described herein may be used as a first therapy, second therapy, third therapy, or combination therapy with other types of cancer therapies known in the art, such as chemotherapy, surgery, hormone therapy, radiation, gene therapy, immunotherapy, bone marrow transplantation, stem cell transplantation, targeted therapy, cryotherapy, ultrasound therapy, photodynamic therapy, radio-frequency ablation or the like, in an adjuvant setting or a neoadjuvant setting (i.e., the method may be carried out before the primary/definitive therapy). In some embodiments, the method is used to treat an individual who has previously been treated. In some embodiments, the cancer has been refractory to prior therapy. In some embodiments, the method is used to treat an individual who has not previously been treated. In some embodiments, the method is for treating blood cancer, such as leukemia or lymphoma, including plasmacytoma and myeloma.

The effective amount of the modified therapeutic cells administered in the methods described herein will depend upon a number of factors, such as the particular type and stage of disease or condition (e.g., cancer) being treated, the route of administrations, the activity of the engineered receptors, and the like. Appropriate dosage regimen can be determined by a physician based on clinical factors, including the patient's size, body surface area, age, the particular compound to be administered, sex, time and route of administration, general health, and other drugs being administered concurrently. In some embodiments, the effective amount of the pharmaceutical composition is below the level that induces a toxicological effect (i.e., an effect above a clinically acceptable level of toxicity) or is at a level where a potential side effect can be controlled or tolerated when the pharmaceutical composition is administered to the individual. In some embodiments, the effective amount of the pharmaceutical composition comprises about 105 to about 10⁹ modified therapeutic cells/kg.

In some embodiments, the pharmaceutical composition is administered for a single time (e.g. bolus injection). In some embodiments, the pharmaceutical composition is administered for multiple times (such as any of 2, 3, 4, 5, 6, or more times). When the pharmaceutical composition is administered for multiple times, each administration may use the same or different routes and may take place at the same site or at alternative sites. The pharmaceutical composition may be administered at a suitable frequency, such as from daily to once per year. The optimal dosage and treatment regime for a particular patient can readily be determined by one skilled in the art of medicine by monitoring the patient for signs of disease and adjusting the treatment accordingly.

In some embodiments, the individual to be treated is a mammal. Examples of mammals include, but are not limited to, humans, monkeys, rats, mice, hamsters, guinea pigs, dogs, cats, rabbits, pigs, sheep, goats, horses, cattle and the like. In some embodiments, the individual is a human.

The modified therapeutic cells are suitable in both autologous cell therapy and allogeneic cell therapy. In some embodiments, the modified therapeutic cell is allogeneic for the individual to be treated. In some embodiments, the modified therapeutic cell has matching MHC allotypes as the individual to be treated. In some embodiments, the modified therapeutic cell has mismatching MHC allotypes as the individual to be treated.

In some embodiments, HLA typing is carried out prior to the treatment methods. In some embodiments, one or more HLA alleles of the individual have mismatching allotypes compared to those of the modified therapeutic cell. In some embodiments, the mismatching alleles are for one or more class I HLA genes. In some embodiments, the mismatching alleles are for one or more class II HLA genes. In some embodiments, no more than any one of 8, 7, 6, 5, 4, 3, 2, or 1 alleles of HLA-A, HLA-B, HLA-C and HLA-DRB1 of the individual have mismatching allotypes compared to those of the modified therapeutic cell. In some embodiments, all tested HLA alleles (e.g., HLA-A, HLA-B, HLA-C and/or HLA-DRB1) of the individual have mismatching allotypes compared to those of the modified therapeutic cell. In some embodiments, all tested HLA alleles (e.g., HLA-A, HLA-B, HLA-C and/or HLA-DRB1) of the individual have matching allotypes compared to those of the modified therapeutic cell.

In some embodiments, HLA antibody screening is carried out prior to the treatment methods. Preformed HLA antibodies can be detected by testing the individual's serum against a panel of lymphocytes with known HLA types. Complement-mediated microlymphocytotoxicity technique or anti-human globulin (AHG) enhancement method can be used for HLA antibody screening. The results are expressed as the percentage of the panel cells that are reactive, which is referred to as percentage panel reactive antibody (% PRA). In some embodiments, the individual has no more than about any one of 90%, 80%, 70%, 60%, 50%, 40%, 30%, 25%, or less PRA.

In some embodiments, crossmatching is performed between the individual and the modified therapeutic cell. In some embodiments, the individual is crossmatch-negative with respect to the modified therapeutic cell. In some embodiments, the individual is crossmatch-positive with respect to the modified therapeutic cell. In some embodiments, the individual has pre-formed antibodies against one or more HLA alleles of the modified therapeutic cell.

In some embodiments, the method reduces undesired immune response associated with the modified therapeutic cell compared to a method using a therapeutic cell that does not comprise the first heterologous nucleic acid sequence encoding the sICP47 protein or functional variant thereof. In some embodiments, the method reduces undesired immune response associated with the modified therapeutic cell in the individual by at least about any one of 10%, 20%, 30%, 40%, 50%, 60%, 70%, 80%, 90% or more. In some embodiments, the undesired immune response comprises HvGD (e.g., T-cell and/or NK-cell mediated HvGD). In some embodiments, the method prevents HvGD (e.g., T-cell and/or NK-cell mediated HvGD) by at least about any one of 10, 20, 30, 40, 50, 60, or more days. In some embodiments, the undesired immune response comprises graft rejection. In some embodiments, the method prevents graft rejection by at least about any one of 10, 20, 30, 40, 50, 60 or more days. In some embodiments, the undesired immune response comprises anti-drug-antibody (ADA) against the modified therapeutic cell. In some embodiments, the method reduces ADA against the modified therapeutic cell by at least about any one of 10%, 20%, 30%, 40%, 50%, 60%, 70%, 80%, 90% or more. In some embodiments, the method induces immune tolerance towards the modified therapeutic cell in the individual compared to a method using a therapeutic cell that does not comprise the first heterologous nucleic acid sequence encoding the sICP47 protein or functional variant thereof. In some embodiments, the method reduces or inhibits NK cell-mediated clearance of the modified therapeutic cell. In some embodiments, the method reduces or inhibits T cell-mediated clearance of the modified therapeutic cell. In some embodiments, the method reduces or inhibits both NK cell-mediated and T cell-mediated clearance of the modified therapeutic cell.

In some embodiments, there is provided a method of reducing graft rejection against allogeneic therapeutic cells in an individual in need thereof, comprising administering to the individual an effective amount of the allogeneic therapeutic cells, wherein the allogeneic therapeutic cells comprise a first heterologous nucleic acid sequence encoding a first heterologous nucleic acid sequence encoding a sICP47 protein or a functional variant thereof. In some embodiments, the therapeutic cells are immune cells, such as cytotoxic T cell, helper T cell, NK cell, NK-T cell, αβ cell, γδ T cell, tumor-infiltrating T cell, dendritic cell (DC)-activated T cell, peripheral blood mononuclear cell (PBMC), or combinations thereof. In some embodiments, the therapeutic cells are stem cells, such as HSC or ESC. In some embodiments, the therapeutic cells further comprise a second heterologous nucleic acid sequence encoding an agonist agent of an NK cell inhibitory receptor (e.g., a membrane-bound anti-NKG2A antibody). In some embodiments, the therapeutic cells further comprise a third heterologous nucleic acid sequence encoding an engineered receptor, such as a CAR, a recombinant TCR, a TAC receptor, and/or a TFP. In some embodiments, the therapeutic cells are CAR-T cells.

At the December 2018 annual meeting of the American Society of Hematology in San Diego, Allogene Therapeutics presented preliminary Phase I data on this UCART19 product. Among 16 evaluable pediatric and young adult patients with acute lymphoblastic leukemia, 88 percent achieved a complete response (CR) with complete or incomplete recovery (CRi) of their blood counts. That is only 14 patients, but in percentage terms roughly in line with the CR/CRi rate of Kymriah. Of the 12 of those patients who achieved minimal residual disease negativity, five remained in remission 4.5 to 16.4 months after receiving UCART19, while seven progressed. Median persistence of the CAR-T cells inside the body was only 28 days. By contrast, an analysis last year found that Kymriah (tisagenlecleucel) persisted for up to 20 months, with a median persistence of 168 days. Of 75 patients with a median follow-up of more than a year, δ1 percent remained in remission (Karen T. M., et al (2018) Clinical Pharmacology of Tisagenlecleucel in B-cell Acute Lymphoblastic Leukemia. Clin Cancer Res; 24(24)). The data of allogeneic CAR-T from Allogene Therapeutics suggest the cells cannot last inside the body as long as their autologous counterparts. As we know, the survival of a certain amount of CAR-T cells in the body plays a decisive role in its efficacy in vivo.

In addition, there are gene knockout methods to remove HLA class I and II molecules from expression on CAR-T cells. For example, by knocking out HLA Class I molecules, the cell membrane that is essential for the expression of β2 microglobulin (B2M), can prevent host TCRab cells from recognizing donor CAR-T cells as heterologous through class I HLA (Poirot, L et al. Cancer Res. 2015, 75, 3853-3864; Valton, J et al. Mol. Ther. 2015, 23, 1507-1518; Torikai, H et al. Blood 2013, 122, 1341-1349; and Liu, X. J. et al. Curr. Res. Transl. Med. 2018, 66, 39-42). However, activated T cells also express class II HLA, which may still stimulate rejection. Even worse, after the HLA molecule is knocked out, the sensitivity of NK cells to cells without HLA expression will be greatly increased. Much shorter persistency of allo-CART is a significant unsolved bottleneck for allo-product to be used as monotherapy.

### Pharmaceutical Compostions

In some embodiments, there is provided a composition comprising any one of the modified therapeutic cells described herein. The composition may comprise any number of the modified therapeutic cell. In some embodiments, the composition comprises a single copy of the modified therapeutic cell. In some embodiments, the composition comprises at least about any of 1, 10, 100, 1000, 10⁴, 10⁵, 10⁶, 10⁷, 10⁸ or more copies of the modified therapeutic cells. In some embodiments, there is provided a pharmaceutical composition comprising an effective amount of modified therapeutic cell (such as allogeneic CAR-T cell), and a pharmaceutically acceptable carrier.

Also provided are the compositions for use in any one of the methods described herein, and use of the compositions in preparation of a medicament for any one of the methods described herein.

“Carriers” as used herein include pharmaceutically acceptable carriers, excipients, or stabilizers which are nontoxic to the cells or individual being exposed thereto at the dosages and concentrations employed. Often the physiologically acceptable carrier is an aqueous pH buffered solution. Examples of suitable pharmaceutical carriers are well known in the art and include phosphate buffered saline solutions, water, emulsions, such as oil/water emulsions, various types of wetting agents, sterile solutions, etc. Acceptable carriers, excipients, or stabilizers are nontoxic to recipients at the dosages and concentrations employed.

Pharmaceutical compositions comprising such carriers can be formulated by well-known conventional methods. The solvent or diluent is preferably isotonic, hypotonic or weakly hypertonic and has a relatively low ionic strength. Representative examples include sterile water, physiological saline (e.g., sodium chloride), Ringer's solution, glucose, trehalose or saccharose solutions, Hank's solution, and other aqueous physiologically balanced salt solutions (see, for example, the most current edition of Remington: The Science and Practice of Pharmacy, A. Gennaro, Lippincott, Williams&Wilkins).

The pharmaceutical compositions described herein may be administered via any suitable routes. In some embodiments, the pharmaceutical composition is administered parenterally, transdermally (into the dermis), intraluminally, intra-arterially (into an artery), intramuscularly (into muscle), intrathecally or intravenously. In some embodiments, the pharmaceutical composition is administered subcutaneously (under the skin). In some embodiments, the pharmaceutical composition is administered intravenously. In some embodiments, the pharmaceutical composition is administered to the individual via infusion or injection. In some embodiments, the pharmaceutical composition is administered directly to the target site, e.g., by biolistic delivery to an internal or external target site or by catheter to a site in an artery. In some embodiments, the pharmaceutical composition is administered locally, e.g., intratumorally. Administrations may use conventional syringes and needles or any compound or device available in the art capable of facilitating or improving delivery of the active agent(s) in the subject.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. In addition, the pharmaceutical composition of the present disclosure might comprise proteinaceous carriers, like, e.g., serum albumin or immunoglobulin, preferably of human origin.

In some embodiments, the pharmaceutical composition is suitably buffered for human use. Suitable buffers include without limitation phosphate buffer (e.g. PBS), bicarbonate buffer and/or Tris buffer capable of maintaining a physiological or slightly basic pH (e.g., from approximately pH 7 to approximately pH 9). In some embodiments, the pharmaceutical composition can also be made to be isotonic with blood by the addition of a suitable tonicity modifier, such as glycerol.

In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container.

In some embodiments, the pharmaceutical composition must meet certain standards for administration to an individual. For example, the United States Food and Drug Administration has issued regulatory guidelines setting standards for cell-based immunotherapeutic products, including 21 CFR 610 and 21 CFR 610.13. Methods are known in the art to assess the appearance, identity, purity, safety, and/or potency of pharmaceutical compositions. In some embodiments, the pharmaceutical composition is substantially free of extraneous protein capable of producing allergenic effects, such as proteins of an animal source used in cell culture other than the therapeutic cells. In some embodiments, “substantially free” is less than about any of 10%, 5%, 1%, 0.1%, 0.01%, 0.001/6, 1 ppm or less of total volume or weight of the pharmaceutical composition. In some embodiments, the pharmaceutical composition is prepared in a GMP-level workshop. In some embodiments, the pharmaceutical composition comprises less than about 5 EU/kg body weight/hr of endotoxin for parenteral administration. In some embodiments, at least about 70% of the therapeutic cells in the pharmaceutical composition are alive for intravenous administration. In some embodiments, the pharmaceutical composition has a “no growth” result when assessed using a 14-day direct inoculation test method as described in the United States Pharmacopoeia (USP). In some embodiments, prior to administration of the pharmaceutical composition, a sample including both the therapeutic cells and the pharmaceutically acceptable excipient should be taken for sterility testing approximately about 48-72 hours prior to the final harvest (or coincident with the last re-feeding of the culture). In some embodiments, the pharmaceutical composition is free of mycoplasma contamination. In some embodiments, the pharmaceutical composition is free of detectable microbial agents. In some embodiments, the pharmaceutical composition is free of communicable disease agents, such as HIV type I, HIV type II, HBV, HCV, Human T-lymphotropic virus, type I; and Human T-lymphotropic virus, type II.

### IV. Kits and Articles of Manufacture

Also provided are kits, unit dosages, and articles of manufacture comprising any one of the modified therapeutic cells described herein.

In some embodiments, a kit is provided which contains any one of the pharmaceutical compositions described herein and preferably provides instructions for its use. In some embodiments, there is provided a kit comprising: (a) any one of the modified therapeutic cells described herein; and (b) instructions for use in any one of the methods described herein. In some embodiments, the modified therapeutic cells are allogeneic therapeutic cells. In some embodiments, the modified therapeutic cells are modified immune cells.

In some embodiments, there is provided a kit comprising: (a) a composition comprising allogeneic CAR-T cells comprising a first heterologous nucleic acid sequence encoding a sICP47 protein or a functional variant thereof; and (b) instructions for treating a disease or condition (e.g., cancer) in an individual in need thereof. In some embodiments, the allogeneic CAR-T cells are universal CAR-T cells. In some embodiments, the allogeneic CAR-T cells target BCMA, or CD19. In some embodiments, the sICP47 protein or functional variant thereof is derived from SA8. In some embodiments, the CAR-T cells further comprise a second heterologous nucleic acid sequence encoding an agonist agent of an NK cell inhibitory receptor, such as a membrane-bound anti-NKG2A antibody.

In some embodiments, the kit further comprises one or more lymphodepletion agents. In some embodiments, the kit further comprises fludarabine and cyclophosphamide. In some embodiments, the kit, in addition to the modified therapeutic cells, further comprises a second cancer therapy, such as chemotherapy, hormone therapy, and/or immunotherapy. The kit(s) may be tailored to a particular cancer for an individual and comprise respective second cancer therapies for the individual.

The kits may contain one or more additional components, such as containers, reagents, culturing media, inducers, cytokines, buffers, antibodies, and the like to allow propagation or induction of the modified therapeutic cells. The kits may also contain a device for administration of the pharmaceutical composition.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like. Some components of the kits may be packaged either in aqueous media or in lyophilized form.

The article of manufacture can comprise a container and a label or package insert on or associated with the container. Suitable containers include, for example, bottles, vials, syringes, etc. The containers may be formed from a variety of materials such as glass or plastic. Generally, the container holds a composition, which is effective for treating a disease, or disorder (such as cancer) described herein, and may have a sterile access port (for example, the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g., from 2-6 administrations). Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

### EXAMPLES

The examples and exemplary embodiments below are intended to be purely exemplary of the invention and should therefore not be considered to limit the invention in any way. The following examples and detailed description are offered by way of illustration and not by way of limitation.

### Example 1: Viral Transfection and Viral Particle Generation

To generate viral particles comprising nucleic acids encoding any of the systems disclosed herein, lentivirus packaging plasmid mixture including pMDLg/pRRE (Addgene #12251), pRSV-Rev (Addgene #12253), and pMD2.G (Addgene #12259) was premixed with a modified PLVX-EF1A vector (including a target construct) at a pre-optimized ratio with polyetherimide (PEI), and incubated at room temperature for 5 minutes. The transfection mix was added dropwise to 293-T cells and mixed gently. Transfected 293-T cells were incubated overnight at 37° C. and 5% CO₂. 24 hours post-transfection, supernatants were collected and centrifuged at 4° C., 500 g for 10 min to remove any cellular debris, followed by an ultracentrifugation step. Centrifuged supernatants were filtered through a 0.45 μm PES filter to concentrate the viral supernatants post ultracentrifugation. After centrifugation, the supernatants were carefully discarded and the virus pellets were rinsed with pre-chilled DPBS. The concentration of virus was measured. Virus was aliquoted and stored at −80° C. Viral titer was determined by functional transduction of a standard T cell line.

Briefly, the lentiviral vector PLVX-EF1A was modified using pLVX-Puro (Clontech #632164) by replacing the original promoter with human elongation factor 1α promoter (hEF1α) and by removing the puromycin resistance gene with EcoRI and BamHI by GenScript. The modified PLVX-EF1A vector contains target construct inserted in the vector. For sICP47 expressing T cells, the target construct was sICP47 sequence-P2A-eGFP. For sICP47 and mNKG2A antibody expressing T cells, the target construct was sICP47 sequence-signal peptide-anti-NKG2A-CD8h/CD8TM-P2A. The CAR used was CD19 CAR. The modified PLVX-EF1A was further subjected to the lentivirus packaging procedure as described above.

### Example 2: Immune Cell Preparation

Leukocytes were collected in R10 medium, and then mixed with 0.9% NaCl solution at a 1:1 (v/v) ratio. 3 mL of lymphoprep medium was added to a 15 mL centrifuge tube. The lymphoprep was slowly layered to form 6 mL of diluted lymphocyte mix. The lymphocyte mix was centrifuged at 800 g for 30 minutes without brakes at 20° C. Lymphocyte buffy coat was then collected with a 200 μL pipette. The harvested fraction was diluted at least 6 fold using 0.9% NaCl or R10 to reduce the density of the solution. The harvested fraction was then centrifuged at 250 g for 10 minutes at 20° C. The supernatant was aspirated completely, and 10 mL of R10 was added to the cell pellet. The mixture was further centrifuged at 250 g for 10 minutes at 20° C. The supernatant was then aspirated. 2 mL of pre-warmed R10 with 100 IU/mL IL-2 at 37° C. was added to the cell pellet, and the cell pellet was re-suspended gently. Cells were quantified and the PBMC sample was ready for experimentation. Human T cells were purified from PBMCs using Miltenyi Pan T cell isolation kit (Cat #130-096-535). Human CD8+ T cells were purified from PBMCs using Miltenyi CD8+ T cell isolation kit (Cat #1 30-094-156). Human NK cells were purified from PBMCs using Miltenyi NK T cell isolation kit (Cat #130-050-401).

The prepared T cells or CD8+ T cells were subsequently pre-activated for 48 hours with human T cell Activation/Expansion kit (Milteny #130-091-441) by using one loaded anti-Biotin MACSiBead Particle per two cells (bead-to-cell ratio 1:2).

### Example 3: T Cell Transfection

The pre-activated T cells were collected and re-suspended in 1640 medium containing 300 IU/mL IL-2. Lentiviral vectors encoding the constructs of was diluted to MOI=5 with the same medium and used to infect 10⁶ activated T cells. The pre-activated T cells were transduced with lentivirus stock in the presence of 8 pg/ml polybrene by centrifugation at 1000 g, 32° C. for 1 hour. The transduced cells containing vectors expressing the target constructs were then transferred to the cell culture incubator for transgene expression under suitable conditions. The transduced cells were centrifuged and replaced with fresh media the following day. Cell density was measured every other day, and fresh media were added to continue the expansion.

### Example 4: B2M Knockout on Primary T Cells by CRWASPR/Cas9 Genome Editing

On days 1-2 of T cells activation, the T cells were collected by centrifugation at 200 g for 10 min. Cells were washed with 2 ml of DPBS and re-suspended for determination of cell density. The cell density was adjusted to 1×10⁶-4×10⁶ per 5 ml in a 15 ml centrifuge tube, followed by centrifugation at 200 g for 10 min. The supernatant was removed after centrifugation. Cas9 solution (made by GenScript) and RNP complex solution (premixed) were added to the cell, and the mixture was incubated for 20 min at room temperature.

The solution for electroporation was prepared as suggested by the kit manual (Human T cell NUCLEOFECTOR™ kit). The electroporation solution was gently added to the cells and incubated for 10 min at room temperature. After incubation, the cell suspension was transferred into an electric rotor, and subjected to the electroporation procedure. After electroporation, 2 ml of pre-warmed medium was added to the cells. The cells were then incubated at 37° C. and 5% CO₂.

To prepare anti-NKG2A antibody expressing B2M knockout T cells, perform transduction of anti-NKG2A expression vector following procedures in Example 3 on day 2-4 after electroporation.

### Example 5: Quantification of Receptor Expression

On day 3 and onwards (typically day 3 to day 8) post transduction, cells were evaluated for expression of the constructs by flow cytometry. An aliquot of cells was collected from the culture, washed, pelleted, and re-suspended in 100 μl PBS, supplemented with 0.5% FBS and diluted binding antibody or antigen protein at a dilution factor of 100. Re-suspended cells were in about 100 μl of Ab. Cells were incubated at 4° C. for 30 minutes. Viability dye was also added according to manufacturer's instructions. After incubation, cells were washed twice in PBS and re-suspended in 100 to 200 μl PBS for analysis. The mean fluorescence of the system was quantified by flow cytometry.

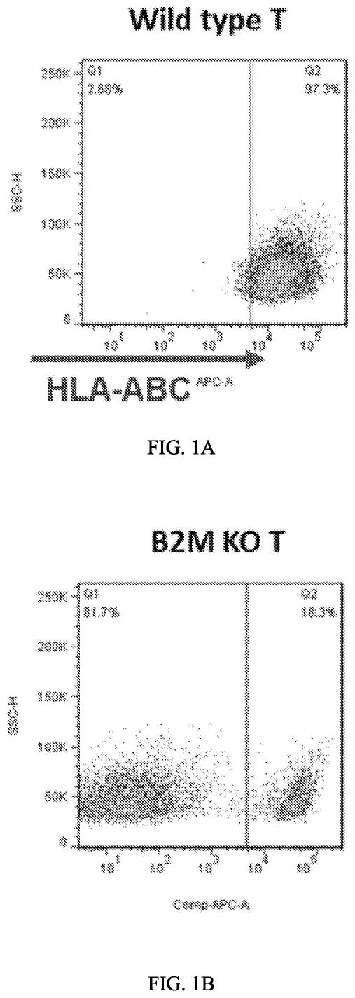

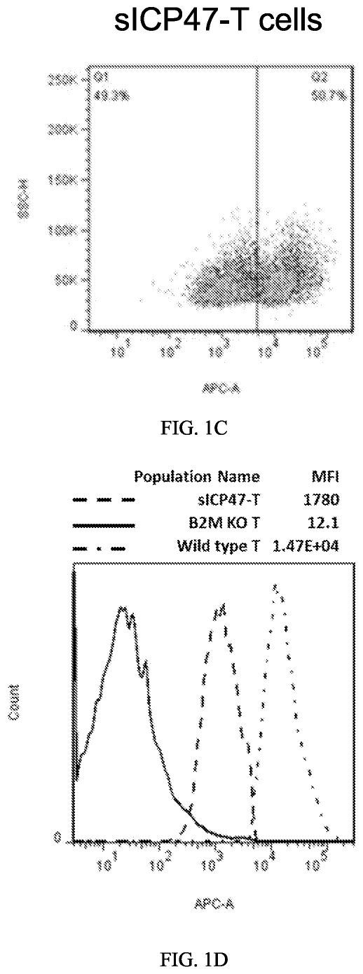

The HLA-ABC expression levels were measured on wild type T cells, sICP47 SA8-T cells, and B2M knockout T cells, as shown in FIGS. 1A-1D. The HLA-ABC expression was down-regulated in sICP47 SA8-T cells. There was no detectable B2M on the cell surface of the B2M knockout T cells.

The HLA-ABC expression levels were measured on wild type T cells, sICP47 SA8-T cells, sICP47 CeHV-16-T cells, sICP47 CeHV-1-T cells, and ICP47 HHV2H-T cells, as shown in FIG. 7. The HLA-ABC expression was down-regulated in sICP47 SA8-T cells, sICP47 CeHV-16-T cells, sICP47 CeHV-1-T cells, and ICP47 HHV2H-T cells.

### Example 6: Allo-Reaction of Allogeneic Wild Type T Cells or Allogeneic CD8+ T Cells with Target Cells

Target cells (B2M knockout T cells and sICP47 SA8-T cells) were labeled with 2.5 uM CFSE in PBS for 5 min at room temperature before the reaction was stopped by addition of 1/10 volume FBS. Cells were washed twice and resuspended in culture media. Effector cells (allo-wild type T cells and allo-CD8+ T cells) and target cells were co-cultured at desired Effector:Target ratios (E:T ratio) for 24 hours or 48 hours. Killing of CFSE labeled target cells were examined by flow cytometry.

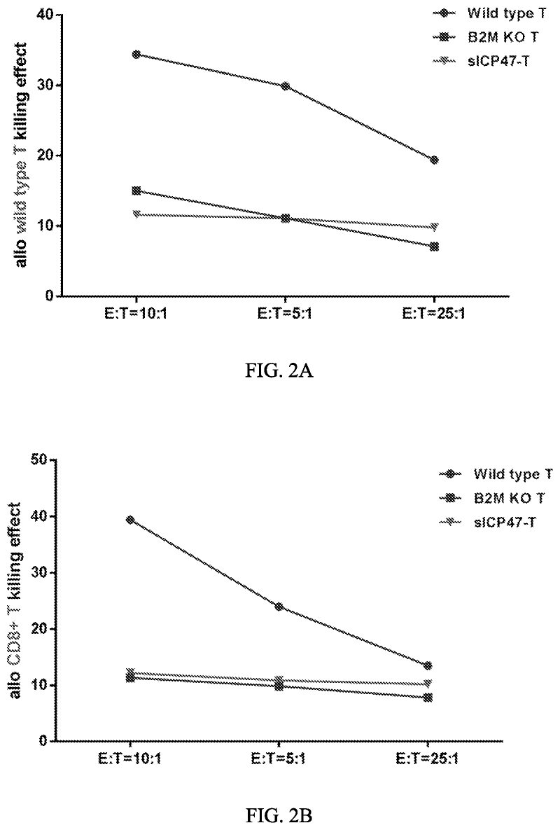

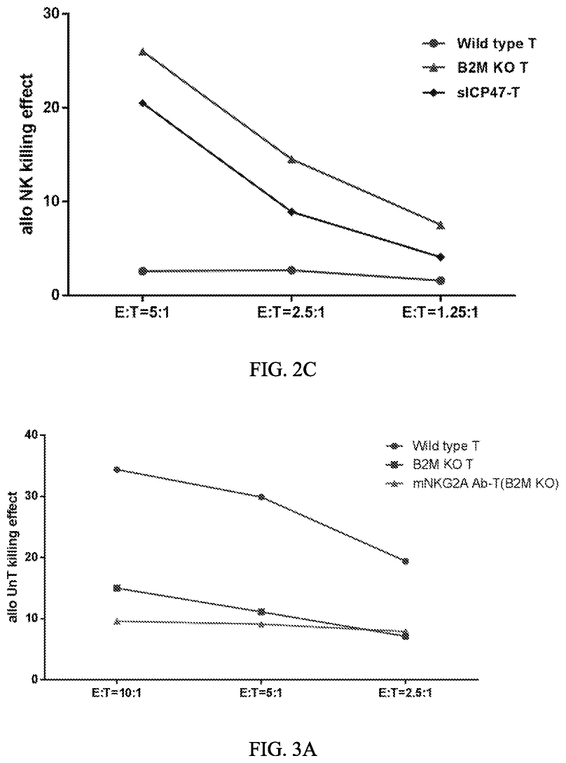

As shown in FIGS. 2A-2B, when allogeneic wild type T cells or allogeneic CD8+ T cells to were co-cultured with the wild type T, B2M knockout T cells or sICP47 SA8-T cells, B2M knockout T cells and sICP47 SA8-T cells showed anti-alloreaction effect as compared to wild type T cells.

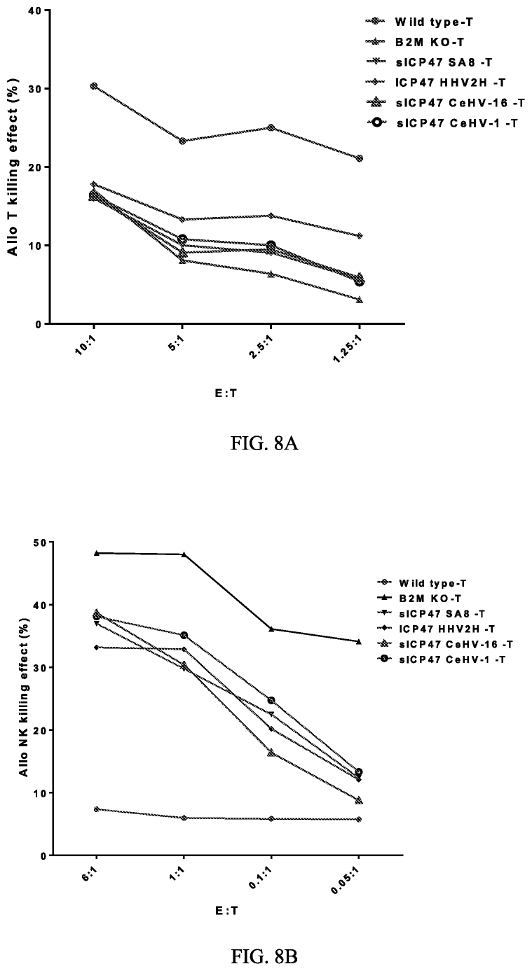

As shown in FIG. 8A, when allogeneic wild type T cells were co-cultured with the wild type T, B2M knockout T cells or sICP47 SA8-T cells, sICP47 CeHV-16-T cells, sICP47 CeHV-1-T cells, and ICP47 HHV2H-T cells showed anti-alloreaction effect as compared to wild type T cells. Compared to ICP47 HHV2H-T cells, sICP47 SA8-T cells, sICP47 CeHV-16-T cells and sICP47 CeHV-1-T cells showed enhanced anti-alloreaction effect when co-cultured with wild type T cells.

### Example 7: Allo-Reaction of Allogeneic NK Cells with Target Cell

Target cells (sICP47-T cells and B2M knockout T cells) were labeled with 2.5 uM CFSE in PBS for 5 min at room temperature before the reaction was stopped by the addition of 1/10 volume FBS. Cells were washed twice and were resuspended in culture media. Effector cells (allo-NK cells) and target cells were co-cultured at desired Effector:Target ratios (E:T ratio) for 24 hours. Killing of CFSE labeled target cells were examined by flow cytometry.

As shown in FIG. 2C. When allogeneic NK cells were co-cultured with the wild type T, B2M knockout T cells, or sICP47 SA8-T cells, NK cells show strong killing effect on the B2M knockout T cells; notably, however, sICP47 SA8-T cells exhibit anti-alloreaction effect when co-cultured with allogeneic NK cells.

As shown in FIG. 8B, when allogeneic NK cells were co-cultured with the wild type T, B2M knockout T cells or sICP47 SA8 transduced T cells, sICP47 CeHV-16-T cells, sICP47 CeHV-1-T cells, and ICP47 HHV2H-T cells showed anti-alloreaction effect as compared to wild type T cells. All these sICP47-T cells exhibit anti-alloreaction effect when co-cultured with allogeneic NK cells. Compared to ICP47 HHV2H-T cells, sICP47 SA8-T cells, sICP47 CeHV-16-T cells and sICP47 CeHV-1-T cells showed similar anti-alloreaction effect when co-cultured with allogeneic NK cells.

### Example 8: Allo-Reaction of Allogeneic NK Cells, Allo-Wild Type T Cells, and all-CD8+ T Cells with Target Cells Expressing NK Inhibitory Receptor Ligand

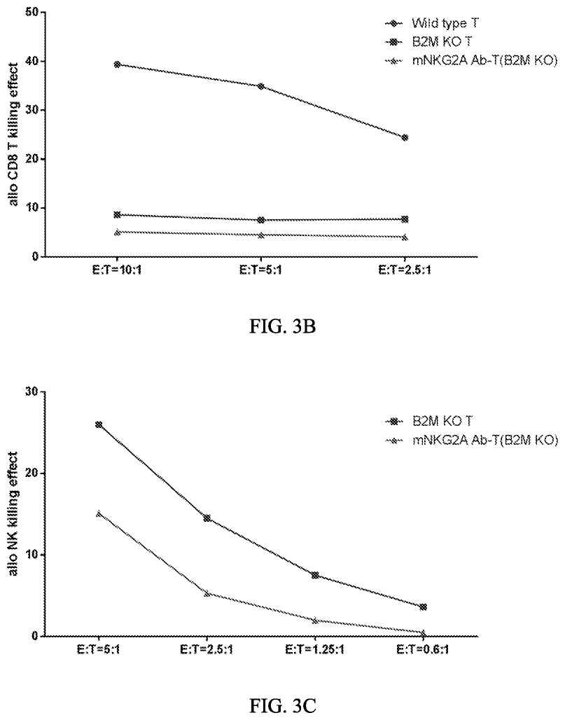

Inhibition of allo-reactive NK cells, allo-wild type T cells, and allo-CD8+ T cells by overexpression of NK inhibitory receptor ligand was tested on membrane bound anti-NKG2A antibody expressing B2M knockout T cells following the procedures in Examples 6 and 7 above. The anti-NKG2A antibody expressing B2M knockout T cells can escape killing by allo-wild type T cells and allo-CD8+ T cells, and can partially inhibit killing by allo-NK cells (FIGS. 3A-3C).

### Example 9: Combinatorial Reduction of Allo-Reaction In Vitro by Co-Expressing sICP47 and Anti-NKG2A Antibody on CAR-T Cells

To test the combinatorial effect of sICP47 and anti-NKG2A antibody on the reduction of allo-reaction, sICP47 SA8 and membrane bound anti-NKG2A antibody were co-expressed on CD19 CAR-T cells using methods in Example 1. The modified T cells herein are denoted as sICP47 SA8+mNKG2A antibody+CD19 CAR-T cells.

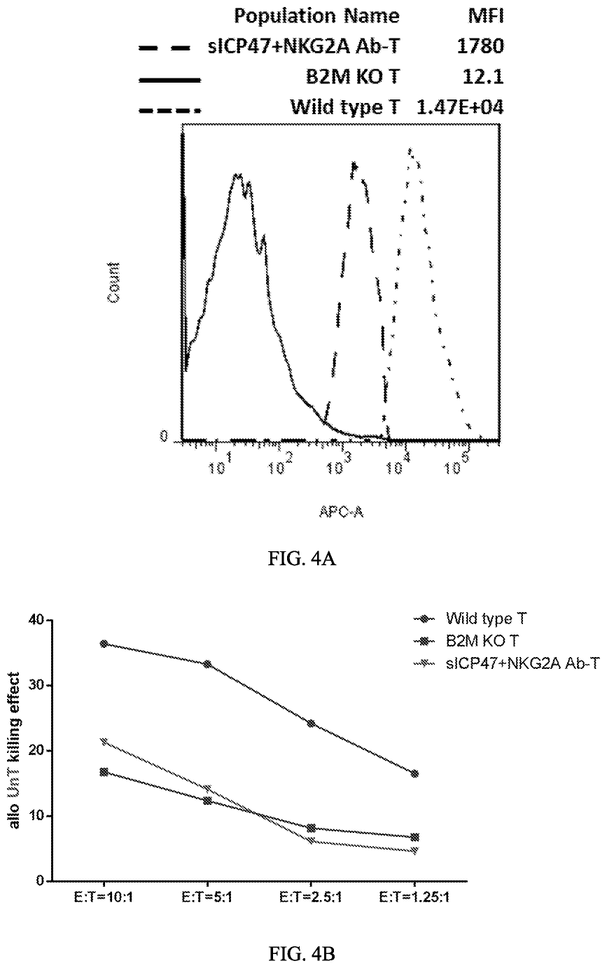

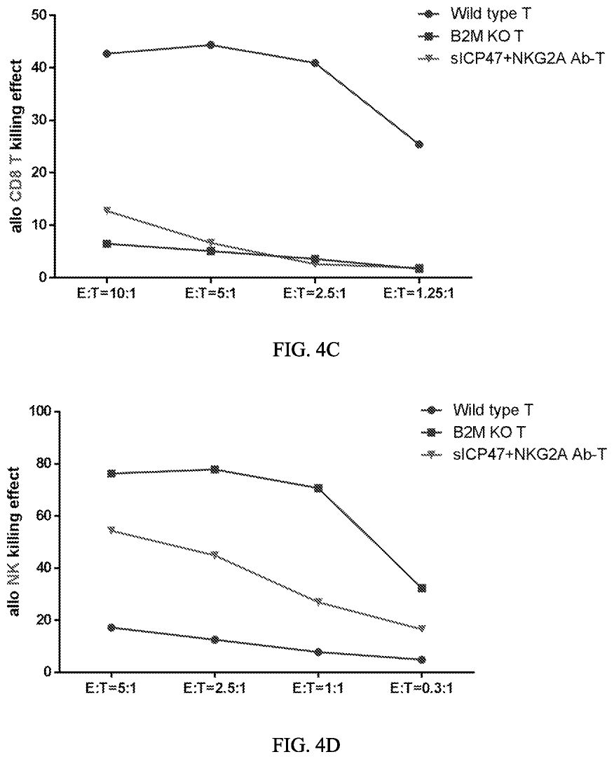

The HLA-ABC expression level on sICP47 SA8+mNKG2A antibody+CD19 CAR-T cells was determined using methods in Example 5. Similar to sICP47 SA8-T cells, the sICP47 SA8+mNKG2A antibody+CD19 CAR-T cells showed down-regulation of HLA-ABC expression level (FIG. 4A).

Inhibition of allo-reactions by the sICP47 SA8+mNKG2A antibody+CD19 CAR-T cells was tested in vitro following the procedures in Examples 6, 7, and 8. Notably, the anti-NKG2A antibody expressing B2M knockout T cells exhibit anti-alloreaction effect against killing by allo-wild type T cells and allo-CD8+ T cells, and can partially inhibit killing by allo-NK cells comparing to B2M knockout T cells (FIGS. 4B-4D).

### Example 10: Combinatorial Reduction of Allo-Reaction In Vivo Using the sICP47+mNKG2A Antibody+CD19 CAR-T Cells

Inhibition of allo-reactions by the sICP47 SA8+mNKG2A antibody+CD19 CAR-T cells was tested in vivo in an HVG model. NCG mice were infused with different combinations of allogeneic cells (PBMCs or NK cells) and graft CAR-T cells, as shown in different groups in Table 3.

TABLE 3
Experimental design

| | Number | Host cells (HLA-A2+) | | | Graft cells (HLA-A2−) | | | |
|---|---|---|---|---|---|---|---|---|
| | of | Infusion | Host | PBMC/ | Infusion | | | |
| Group | mice | time | cells | NK dose | time | Graft T cells | | Dose |
| 1 | 4 | Day-4 | — | — | Day 0 | Donor 2 | UnT | 5M |
| 2 | 4 | Day-4 | Allo-PBMC | 20M | Day 0 | Donor 2 | UnT | 5M |
| 3 | 4 | Day-4 | — | — | Day 0 | Donor 2 | B2M KO T | 5M |
| 4 | 4 | Day-4 | Allo-PBMC | 20M | Day 0 | Donor 2 | B2M KO T | 5M |
| 5 | 4 | Day-4 | — | — | Day 0 | Donor 2 | sICP47 SA8 + | 5M |
| | | | | | | | mNKG2A | |
| | | | | | | | antibody + | |
| | | | | | | | CD19 | |
| | | | | | | | CAR-T | |
| 6 | 4 | Day-4 | Allo-PBMC | 20M | Day 0 | Donor 2 | sICP47 SA8 + | 5M |
| | | | | | | | mNKG2A | |
| | | | | | | | antibody + | |
| | | | | | | | CD19 | |
| | | | | | | | CAR-T | |
| 7 | 3 | Day-4 | Allo-PBMC | 20M | Day 0 | Donor 2 | TRAC KO T | 5M |
| 8 | 4 | Day-4 | Allo-NK | 10M * 3 | Day 0, 3, 6 | Donor 2 | — | — |
| 9 | 4 | Day-4 | Allo-NK | 10M * 3 | Day 0, 3, 6 | Donor 2 | UnT | 5M |
| 10 | 4 | Day-4 | Allo-NK | 10M * 3 | Day 0, 3, 6 | Donor 2 | B2M KO T | 5M |
| 11 | 4 | Day-4 | Allo-NK | 10M * 3 | Day 0, 3, 6 | Donor 2 | sICP47 SA8 + | 5M |
| | | | | | | | mNKG2A | |
| | | | | | | | antibody + | |
| | | | | | | | CD19 | |
| | | | | | | | CAR-T | |

### Model I: Allo-PBMC as Host Cells in NCG Mice

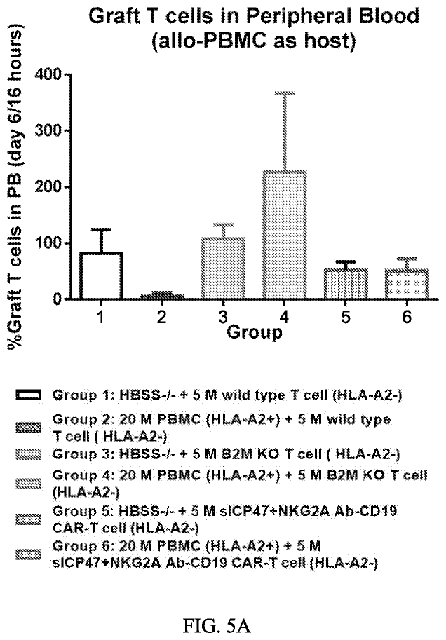

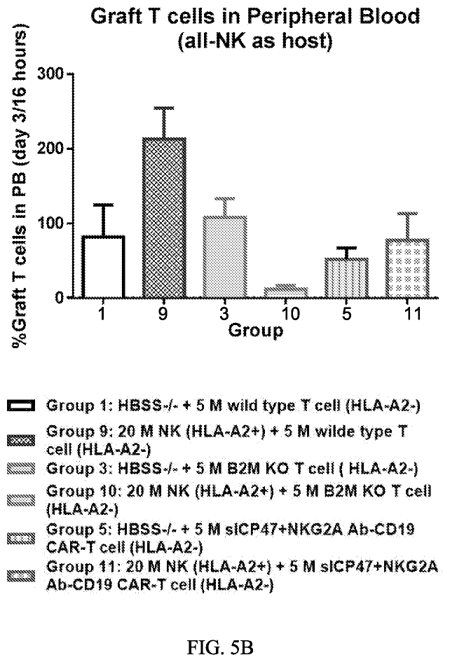

4 days (denoted as day −4 in Table 3 and hereafter) prior to injecting graft Tcells, 20 M PBMC cells were intravenously injected into the NCG mice in groups 2, 4, 6, and 7 as host cells to reconstitute the humanized immune-system. Then 5 M allogenic unT, or B2M KO T, or TRAC KO T or sICP47 SA8+mNKG2A antibody+CD19 CAR-T cells were intravenously injected to the mice 4 days after host cell injection (denoted as day 0), respectively. To distinguish host cells from graft T cells, we used the cells with different HLA-A2 subtype (sec Table 3): the host cells are HLA-A2+, and the graft Tcells are HLA-A2−. After grafted cell infusion, we performed the FACS to detect host Tcells, host NK cells and grafted T cells in the peripheral blood weekly, calculated the percentage of each cell type by ratio of cell number to total live cells, and the survival percentage of each cell type by ratio of live cells at the time point of counting to the number of cells infused. If the animal displayed serious GvHD signs, or the body weight loss>20%, we humanely euthanized the mouse by CO₂ followed by cervical dislocation. 6 days and 16 hours after cell infusion, host T cells and graft T cells were collected from the peripheral blood and counted. Results are shown in FIG. 5A.

Cell counts of PBMC host cells expanded from 1.46% to 17.03% in peripheral blood. NK cells, on the other hand, decreased from 1.51% to 0.77%, as they cannot survive without supplement of cytokines in this model. These data indicate that host T cells are the major host immune cells this model. When host PBMC is absent (Group 1, FIG. 5A), 81.75% of the wild type T cells remained in the peripheral blood at 6 days and 16 hours after cell infusion. In contrast, wild type T cells were rejected in the presence of host PBMC (Group 2, FIG. 5A), with only 6% wild type T cells remaining.

A comparison between Groups 3 and 4 shows that the B2M knockout T cells in the absence of host PBMC persisted in the peripheral blood (107.75% of the amount infused in NCG mice, Group 3, FIG. 5A); and in the presence of host PBMC, the B2M knockout T cells proliferated in the peripheral blood (226.5%, Group 4, FIG. 5A), possibly due to allo-reaction/stimulation between the host T cells and the graft T cells as in the case of GVHD. Notably, 51.8% of the sICP47+mNKG2A antibody+CD19 CAR-T cells in Group 5 (without host PBMC) and a similar percentage in Group 6 (with PBMC) were present in the peripheral blood (FIG. 5A).

### Model II: Activated Allo-NK Cells as Hast Cells in NCG Mike

Next, the graft T cells were subjected to allogeneic NK cells as host cells in NCG mice. As shown in Table 3, 10 M cultured and purified NK cells were intravenously injected into the NCG mice in Groups 8, 9, 10, and 11 on day 4. Moreover, 3 days and 16 hours of after infusion, cells were collected from peripheral blood and counted. Results are shown in FIG. 5B.

The wild type T cells in Group 9 proliferated to 213% of the infused amount in the presence of host NK cells. B2M knockout T cells are prone to killing by the NK cells, and only 11.9% of the grafted B2M knockout T cells (Group 10, FIG. 5B) remained in the peripheral blood in the presence of host NK cells. Remarkably, 77.4% the sICP47 SA8+mNKG2A antibody+CD19 CAR expressing T cells in Group 11 (FIG. 5B, with host NK cells) remained, higher than the 51.8% remaining in Group 5 (without host T cells or NK cells). These results showed that the sICP47+mNKG2A antibody+CD19 CAR expressing T cells proliferated in the peripheral blood with host NK cells present and demonstrated better persistence in vivo compared to B2M knockout T cells.

SEQUENCE LISTING

SEQ ID NO: 1 sICP47 active domain of SA8
SLYLATVDAFLRNPHTRHRTCADLRRELDAYADE
SEQ ID NO:2 sICP47 active domain of CeHV-16
SLYLAEVDAFLQSPRTRHRTCADLRRELDAYADE
SEQ ID NO: 3 sICP47 active domain of CeHV-1
SRYLAAVDDYLHHPSPRYQAHVDLRRELRAYADE
SEQ ID NO: 4 sICP47 of SA8
MSSLYLATVDAFLRNPHTRHRTCADLRRELDAYADEERREAAKAIAHPDRPLLAPPSAP
PNHSHLAARETAPPPAATP
SEQ ID NO: 5 sICP47 of CeHV-16
MSSLYLAEVDAFLQSPRTRHRTCADLRRELDAYADEERREAAKAIAHPDRPLLAPPSAP
PDRSRPAPRGTAHPPAASP
SEQ ID NO: 6 sICP47 of CeHV-1
MSSRYLAAVDDYLHHPSPRYQAHVDLRRELRAYADEERREAARAIAHPERPLLPPPATQ
AAPPQPSTREAAHPSAPTAASS
SEQ ID NO: 7 anti-NKG2A Ab HC-CDR1
GYTFTSYWMN
SEQ ID NO: 8 anti-NKG2A Ab HC-CDR2
RIDPYDSETHYAQKLQG
SEQ ID NO: 9 anti-NKG2A Ab HC-CDR3
GGYDFDVGTLYWFFDV
SEQ ID NO: 10 anti-NKG2A Ab LC-CDR1
RASENIYSYLA
SEQ ID NO: 11 anti-NKG2A Ab LC-CDR2
NAKTLAE
SEQ ID NO: 12 anti-NKG2A Ab LC-CDR3
QHHYGTPRT
SEQ ID NO: 13 anti-NKG2A Ab VH
QVQLVQSGAEVKKPGASVKVSCKASGYTFTSYWMNWVRQAPGQGLEWMGRIDPYDS
ETHYAQKLQGRVTMTTDTSTSTAYMELRSLRSDDTAVYYCARGGYDFDVGTLYWFFD
VWGQGTTVTVSS
SEQ ID NO: 14 anti-NKG2A Ab VL
DIQMTQSPSSLSASVGDRVTITCRASENIYSYLAWYQQKPGKAPKLLIYNAKTLAEGVPS
RFSGSGSGTDFTLTISSLQPEDFATYYCQHHYGTPRTFGGGTKVEIK
SEQ ID NO: 15 anti-NKG2A scFv
DIQMTQSPSSLSASVGDRVTITCRASENIYSYLAWYQQKPGKAPKLLIYNAKTLAEGVPS
RFSGSGSGTDFTLTISSLQPEDFATYYCQHHYGTPRTFGGGTKVEIKGGGGSGGGGSGG
GGSQVQLVQSGAEVKKPGASVKVSCKASGYTFTSYWMNWVRQAPGQGLEWMGRIDP
YDSETHYAQKLQGRVTMTTDTSTSTAYMELRSLRSDDTAVYYCARGGYDFDVGTLYW
FFDVWGQGTTVTVSS
SEQ ID NO: 16 membrane bound anti-NKG2A
MALPVTALLLPLALLLHAARPDIQMTQSPSSLSASVGDRVTITCRASENIYSYLAWYQQ
KPGKAPKLLIYNAKTLAEGVPSRFSGSGSGTDFTLTISSLQPEDFATYYCQHHYGTPRTF
GGGTKVEIKGGGGSGGGGSGGGGSQVQLVQSGAEVKKPGASVKVSCKASGYTFTSYW
MNWVRQAPGQGLEWMGRIDPYDSETHYAQKLQGRVTMTTDTSTSTAYMELRSLRSDD
TAVYYCARGGYDFDVGTLYWFFDVWGQGTTVTVSSTTTPAPRPPTPAPTIASQPLSLRP
EACRPAAGGAVHTRGLDFACDIYIWAPLAGTCGVLLLSLVITLYC
SEQ ID NO: 17 CD8a hinge
TTTPAPRPPTPAPTIASQPLSLRPEACRPAAGGAVHTRGLDFACD
SEQ ID NO: 18 CD8a TM
IYIWAPLAGTCGVLLLSLVITLYC
SEQ ID NO: 19 ICP47-HHV11
MSWALEMADTFLDTMRVGPRTYADVRDEINKRGREDREAARTAVHDPERPLLRSPGLL
PEIAPNASLGVAHRRTGGTVTDSPRNPVTR
SEQ ID NO: 20 ICP47-HHV2H
MSWALKTTDMFLDSSRCTHRTYGDVCAEIHKREREDREAARTAVTDPELPLLCPPDVRS
DPASRNPTQQTRGCARSNERQDRVLAP
SEQ ID NO: 21 CD19 CAR
MALPVTALLLPLALLLHAARPDIQMTQTTSSLSASLGDRVTISCRASQDISKYLNWYQQ
KPDGTVKLLIYHTSRLHSGVPSRFSGSGSGTDYSLTISNLEQEDIATYFCQQGNTLPYTFG
GGTKLEITGGGGSGGGGSGGGGSEVKLQESGPGLVAPSQSLSVTCTVSGVSLPDYGVSW
IRQPPRKGLEWLGVIWGSETTYYNSALKSRLTIIKDNSKSQVFLKMNSLQTDDTAIYYCA
KHYYYGGSYAMDYWGQGTSVTVSSTTTPAPRPPTPAPTIASQPLSLRPEACRPAAGGAV
HTRGLDFACDIYIWAPLAGTCGVLLLSLVITLYCKRGRKKLLYIFKQPFMRPVQTTQEED
GCSCRFPEEEEGGCELRVKFSRSADAPAYKQGQNQLYNELNLGRREEYDVLDKRRGRD
PEMGGKPRRKNPQEGLYNELQKDKMAEAYSEIGMKGERRRGKGHDGLYQGLSTATKD
TYDALHMQALPPR
SEQ ID NO: 22 EGFP
MVSKGEELFTGVVPILVELDGDVNGHKFSVSGEGEGDATYGKLTLKFICTTGKLPVPWP
TLVTTLTYGVQCFSRYPDHMKQHDFFKSAMPEGYVQERTIFFKDDGNYKTRAEVKFEG
DTLVNRIELKGIDFKEDGNILGHKLEYNYNSHNVYIMADKQKNGIKVNFKIRHNIEDGSV
QLADHYQQNTPIGDGPVLLPDNHYLSTQSALSKDPNEKRDHMVLLEFVTAAGITLGMD
ELYK
SEQ ID NO: 23 P2A
GSGATNFSLLKQAGDVEENPGP
SEQ ID NO: 24 sICP47 of Macacine alphaherpesvirus 1
MSSRYLAAADDYLRHPTPRYRTYVDLRKDLDAYADEERRDAAKAIAHPDRPLLLPPPN
PQRISARESAPPSPPTTASH
SEQ ID NO: 25 sICP47 of Pappine alphaherpesvirus 2
MSSLYLAEADAFLQSPHTRHRTCADLRRELDAYADEERREAAKAIAHPDRPLLAPPSAP
PDRSRPAPREAAHPPAASP

What is claimed is:

1. A modified therapeutic cell comprising a first heterologous nucleic acid sequence encoding a simian ICP47 (sICP47) protein or a functional variant thereof.

2. The modified therapeutic cell of claim 1, wherein the first heterologous nucleic acid sequence encodes a naturally occurring sICP47 protein.

3. The modified therapeutic cell of claim 1, wherein the first heterologous nucleic acid sequence encodes an engineered sICP47 protein comprising an active domain of a naturally occurring sICP47 protein.

4. The modified therapeutic cell of claim 2 or 3, wherein the naturally occurring sICP47 protein is from a herpesvirus selected from the group consisting of Simian Agent 8 (SA8), Cercopithecine herpesvirus 16 (CeHV-16) and Cercopithecine herpesvirus 1 (CeHV-1).

5. The modified therapeutic cell of claim 4, wherein the first heterologous nucleic acid sequence encodes a sICP47 of SA8 or a functional variant thereof.

6. The modified therapeutic cell of claim 4, wherein the first heterologous nucleic acid sequence encodes a sICP47 of CeHV-16 or a functional variant thereof.

7. The modified therapeutic cell of claim 4, wherein the first heterologous nucleic acid sequence encodes a sICP47 of CeHV-1 or a functional variant thereof.

8. The modified therapeutic cell of any one of claims 1-7, wherein the sICP47 protein or functional variant thereof comprises an amino acid sequence having at least 80% sequence identity to an amino acid sequence selected from the group consisting of SEQ ID NOs: 1-3.

9. The modified therapeutic cell of claim 8, wherein the sICP47 protein or functional variant thereof comprises the amino acid sequence of SEQ ID NO: 1.

10. The modified therapeutic cell of any one of claims 1-9, wherein the sICP47 protein or functional variant thereof comprises an amino acid sequence having at least 80% sequence identity to an amino acid sequence selected from the group consisting of SEQ ID NOs: 4-6.

11. The modified therapeutic cell of claim 10, wherein the first heterologous nucleic acid sequence encodes a sICP47 protein comprising the amino acid sequence of SEQ ID NO: 4.

12. The modified therapeutic cell of any one of claims 1-11, further comprising a second heterologous nucleic acid sequence encoding an agonist agent of a natural killer (NK) cell inhibitory receptor.

13. The modified therapeutic cell of claim 12, wherein the NK cell inhibitory receptor is an NKG2A receptor.

14. The modified therapeutic cell of claim 12 or 13, wherein the agonist agent is a ligand of the NK cell inhibitory receptor.

15. The modified therapeutic cell of claim 12 or 13, wherein the agonist agent is an agonist antibody or antigen binding fragment thereof that specifically binds to the NK cell inhibitory receptor.

16. The modified therapeutic cell of claim 15, wherein the agonist antibody or antigen binding fragment thereof is membrane bound.

17. The modified therapeutic cell of claim 15 or 16, wherein the agonist antibody or antigen binding fragment thereof specifically binds to NKG2A.

18. The modified therapeutic cell of claim 17, wherein:
(a) the agonist antibody or antigen binding fragment thereof comprises:
(i) a heavy chain variable region (VH) comprising a heavy chain complementary determining region (HC-CDR) 1 comprising the amino acid sequence of SEQ ID NO: 7, an HC-CDR2 comprising the amino acid sequence of SEQ ID NO: 8, and an HC-CDR3 comprising the amino acid sequence of SEQ ID NO: 9; and
(ii) a light chain variable region (VL) comprising a light chain complementary determining region (LC-CDR) 1 comprising the amino acid sequence of SEQ ID NO: 10, an LC-CDR2 comprising the amino acid sequence of SEQ ID NO: 11, and an LC-CDR3 comprising the amino acid sequence of SEQ ID NO: 12; and/or
(b) the agonist antibody or antigen binding fragment thereof comprises a VH comprising an amino acid sequence having at least about 85% sequence identity to the amino acid sequence of SEQ ID NO: 13, and a VL comprising an amino acid sequence having at least about 85% sequence identity to the amino acid sequence of SEQ ID NO: 14.

19. The modified therapeutic cell of claim 17 or 18, wherein the agonist agent is a polypeptide comprising from the N-terminus to the C-terminus a single-chain variable fragment (scFv) that specifically binds to NKG2A, a CD8α hinge sequence and a CD8α transmembrane sequence.

20. The modified therapeutic cell of claim 19, wherein the agonist agent comprises an amino acid sequence having at least about 85% sequence identity to the amino acid sequence of SEQ ID NO: 15.

21. The modified therapeutic cell of claim 19 or 20, wherein the agonist agent comprises an amino acid sequence having at least about 85% sequence identity to the amino acid sequence of SEQ ID NO: 16.

22. The modified therapeutic cell of any one of claims 1-21, wherein the therapeutic cell is an immune cell.

23. The modified therapeutic cell of claim 22, wherein the immune cell is selected from the group consisting of cytotoxic T cell, helper T cell, NK cell, NK-T cell, as T cell, γ5 T cell, tumor-infiltrating T cell, dendritic cell (DC)-activated T cell, macrophage and peripheral blood mononuclear cell (PBMC).

24. The modified therapeutic cell of claim 22, wherein the therapeutic cell is a γ5 T cell, optionally wherein the therapeutic cell is a γ9δ2 T cell, a δ1 T cell, or a δ3 T cell.

25. The modified therapeutic cell of any one of claims 1-21, wherein the therapeutic cell is a stem cell.

26. The modified therapeutic cell of claim 25, wherein the therapeutic cell is an embryonic stem cell.

27. The modified therapeutic cell of claim 25, wherein the therapeutic cell is a hematopoietic stem cell (HSC).

28. The modified therapeutic cell of any one of claims 1-27, further comprising a heterologous nucleic acid sequence encoding an engineered receptor.

29. The modified therapeutic cell of claim 28, wherein the engineered receptor is selected from the group consisting of a chimeric antigen receptor (CAR), a recombinant T cell receptor (TCR), a T-cell antigen coupler (TAC) receptor and a TCR fusion protein (TFP).

30. The modified therapeutic cell of claim 29, wherein the therapeutic cell is a CAR-T cell.

31. The modified therapeutic cell of any one of claims 28-30, wherein the engineered receptor targets a tumor antigen selected from the group consisting of CD19, BCMA, NY-ESO-1, VEGFR2, MAGE-A3, CD20, CD22, CD33, CD38, CEA, EGFR, GD2, HER2, IGF1R, mesothelin, PSMA, ROR1, WT1, GPC3, AFP, CD30, CD4 and combinations thereof.

32. The modified therapeutic cell of claim 31, wherein the engineered receptor is an anti-BCMA CAR or anti-CD19 CAR.

33. The modified therapeutic cell of any one of claims 1-32, wherein the Major Histocompatibility Complex (MHC) genes of the therapeutic cell are not genetically modified.

34. The modified therapeutic cell of any one of claims 1-33, wherein expression of the sICP47 protein or functional variant thereof downregulates cell surface expression of MHC molecules in the therapeutic cell.

35. A method of producing a modified therapeutic cell, comprising introducing into a precursor cell a first heterologous nucleic acid sequence encoding a sICP47 or a functional variant thereof.

36. A modified therapeutic cell obtained by the method of claim 35.

37. A pharmaceutical composition comprising the modified therapeutic cell of any one of claims 1-34 and 36, and a pharmaceutically acceptable carrier.

38. A method of treating a disease or condition in an individual in need thereof, comprising administering to the individual an effective amount of the pharmaceutical composition of claim 37.

39. The method of claim 38, wherein the modified therapeutic cell is allogeneic.

40. The method of claim 38 or 39, wherein the individual is human.

41. The method of claim 40, wherein one or more HLA alleles of the individual have mismatching allotypes compared to those of the modified therapeutic cell, optionally wherein one or more alleles of HLA-A, HLA-B, HLA-C and HLA-DRB1 mismatching allotypes compared to those of the modified therapeutic cell.

42. The method of any one of claims 38-41, wherein the method reduces undesired immune response against the therapeutic cell in the individual compared to a method using a therapeutic cell that does not comprise the first heterologous sequence encoding the sICP47 protein or functional variant thereof.

43. The method of claim 42, wherein the undesired immune response comprises Host-versus-Graft (HvG) response.

44. The method of claim 43, wherein the HvG response is mediated by T cells of the individual.

45. The method of claim 43 or 44, wherein the HvG response is mediated by NK cells of the individual.

46. The method of any one of claims 38-45, wherein: (i) the method induces immune tolerance towards the therapeutic cell in the individual compared to a method using a therapeutic cell that does not comprise the first heterologous nucleic acid sequence encoding the sICP47 protein or functional variant thereof; and/or the (ii) method increases persistence of the therapeutic cell in the individual compared to a method using a therapeutic cell that does not comprise the first heterologous nucleic acid sequence encoding the sICP47 protein or functional variant thereof.

47. The method of any one of claims 38-46, wherein the disease or condition is a cancer, an autoimmune disease, or an infectious disease.

48. A method of reducing graft rejection of allogeneic therapeutic cells in an individual in need thereof, comprising administering to the individual an effective amount of the allogeneic therapeutic cells, wherein the allogeneic therapeutic cells comprise a first heterologous nucleic acid sequence encoding a sICP47 protein or functional variant thereof.

49. A method of down-regulating expression of MHC class I molecule on a modified therapeutic cell, comprising introducing into a precursor cell a first heterologous nucleic acid sequence encoding a sICP47 or a functional variant thereof.

50. A kit comprising the modified therapeutic cell of any one of claims 1-34 and 36 and instructions for treating a disease or condition in an individual in need thereof.


SEQUENCE LISTING (25 sequences)

SEQ ID NO: 1 - protein, 34 aa, Artificial Sequence
  Synthetic Construct
SLYLATVDAFLRNPHTRHRTCADLRRELDAYADE

SEQ ID NO: 2 - protein, 34 aa, Artificial Sequence
  Synthetic Construct
SLYLAEVDAFLQSPRTRHRTCADLRRELDAYADE

SEQ ID NO: 3 - protein, 34 aa, Artificial Sequence
  Synthetic Construct
SRYLAAVDDYLHHPSPRYQAHVDLRRELRAYADE

SEQ ID NO: 4 - protein, 78 aa, Artificial Sequence
  Synthetic Construct
MSSLYLATVDAFLRNPHTRHRTCADLRRELDAYADEERREAAKAIAHPDRPLLAPPSAPPNHSHLAARETAPPPAATP

SEQ ID NO: 5 - protein, 78 aa, Artificial Sequence
  Synthetic Construct
MSSLYLAEVDAFLQSPRTRHRTCADLRRELDAYADEERREAAKAIAHPDRPLLAPPSAPPDRSRPAPRGTAHPPAASP

SEQ ID NO: 6 - protein, 81 aa, Artificial Sequence
  Synthetic Construct
MSSRYLAAVDDYLHHPSPRYQAHVDLRRELRAYADEERREAARAIAHPERPLLPPPATQAAPPQPSTREAAHPSAPTAASS

SEQ ID NO: 7 - protein, 10 aa, Artificial Sequence
  Synthetic Construct
GYTFTSYWMN

SEQ ID NO: 8 - protein, 17 aa, Artificial Sequence
  Synthetic Construct
RIDPYDSETHYAQKLQG

SEQ ID NO: 9 - protein, 16 aa, Artificial Sequence
  Synthetic Construct
GGYDFDVGTLYWFFDV

SEQ ID NO: 10 - protein, 11 aa, Artificial Sequence
  Synthetic Construct
RASENIYSYLA

SEQ ID NO: 11 - protein, 7 aa, Artificial Sequence
  Synthetic Construct
NAKTLAE

SEQ ID NO: 12 - protein, 9 aa, Artificial Sequence
  Synthetic Construct
QHHYGTPRT

SEQ ID NO: 13 - protein, 125 aa, Artificial Sequence
  Synthetic Construct
QVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSS

SEQ ID NO: 14 - protein, 107 aa, Artificial Sequence
  Synthetic Construct
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIK

SEQ ID NO: 15 - protein, 247 aa, Artificial Sequence
  Synthetic Construct
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIKGGGGSGGGGSGGGGSQVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSS

SEQ ID NO: 16 - protein, 337 aa, Artificial Sequence
  Synthetic Construct
MALPVTALLLPLALLLHAARPDIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIKGGGGSGGGGSGGGGSQVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSSTTTPAPRPPTPAPTIASQPLSLRPEACRPAAGGAVHTRGLDFACDIYIWAPLAGTCGVLLLSLVITLYC

SEQ ID NO: 17 - protein, 45 aa, Artificial Sequence
  Synthetic Construct
TTTPAPRPPTPAPTIASQPLSLRPEACRPAAGGAVHTRGLDFACD

SEQ ID NO: 18 - protein, 24 aa, Artificial Sequence
  Synthetic Construct
IYIWAPLAGTCGVLLLSLVITLYC

SEQ ID NO: 19 - protein, 88 aa, Artificial Sequence
  Synthetic Construct
MSWALEMADTFLDTMRVGPRTYADVRDEINKRGREDREAARTAVHDPERPLLRSPGLLPEIAPNASLGVAHRRTGGTVTDSPRNPVTR

SEQ ID NO: 20 - protein, 86 aa, Artificial Sequence
  Synthetic Construct
MSWALKTTDMFLDSSRCTHRTYGDVCAEIHKREREDREAARTAVTDPELPLLCPPDVRSDPASRNPTQQTRGCARSNERQDRVLAP

SEQ ID NO: 21 - protein, 486 aa, Artificial Sequence
  Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 239 aa, Artificial Sequence
  Synthetic Construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 23 - protein, 22 aa, Artificial Sequence
  Synthetic Construct
GSGATNFSLLKQAGDVEENPGP

SEQ ID NO: 24 - protein, 78 aa, Macacine alphaherpesvirus 1
MSSRYLAAADDYLRHPTPRYRTYVDLRKDLDAYADEERRDAAKAIAHPDRPLLLPPPNPQRISARESAPPSPPTTASH

SEQ ID NO: 25 - protein, 78 aa, Pappine alphaherpesvirus 2
MSSLYLAEADAFLQSPHTRHRTCADLRRELDAYADEERREAAKAIAHPDRPLLAPPSAPPDRSRPAPREAAHPPAASP